(19) United States
(12) Patent Application Publication
(10) Pub. No.: US 2016/0102058 A1
(43) Pub. Date: Apr. 14, 2016

(54) FLUORO-PERHEXILINE COMPOUNDS AND THEIR THERAPEUTIC USE

(71) Applicant: THE UNIVERSITY COURT OF THE UNIVERSITY OF ABERDEEN, Aberdeenshire (GB)

(72) Inventors: Iain Robert Greig, Aberdeen, Aberdeenshire (GB); Matteo Zanda, Aberdeen Aberdeenshire (GB); Chih-Chung Tseng, Aberdeen, Aberdeenshire (GB)

(21) Appl. No.: 14/890,462

(22) PCT Filed: Nov. 20, 2014
(86) PCT No.: PCT/GB2014/051488

§ 371 Date: Nov. 11, 2015

(30) Foreign Application Priority Data
May 15, 2013 (GB) 1308736.6

(51) Int. Cl. C07D 211/18

(52) U.S. Cl. CPC C07D 211/18

(57) ABSTRACT

The present invention pertains generally to the field of therapeutic compounds. More specifically the present invention pertains to certain fluoro-perhexiline compounds of the following formula (also referred to herein as FPER compounds) that are useful, for example, in the treatment of disorders (e.g., diseases) including, for example, those which are known to be treated with, or known to be treatable with, perhexiline, including, for example, disorders that are ameliorated by the inhibition of carnitine palmitoyltransferase (CPT); cardiovascular disorders such as: angina pectoris; heart failure (HF); ischaemic heart disease (IHD); cardiomyopathy; cardiac dysrhythmia; stenosis of a heart valve; hypertrophic cardiomyopathy (HCM); coronary heart disease; and other disorders, for example, diabetes and cancer. The present invention also pertains to pharmaceutical compositions comprising such compounds, and the use of such compounds and compositions, for example, in therapy.

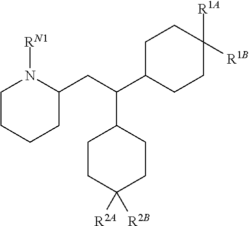

### RELATED APPLICATION

This application is related to United Kingdom patent application number 1308736.6 filed 15 May 2013, the contents of which are incorporated herein by reference in their entirety.

### TECHNICAL FIELD

The present invention pertains generally to the field of therapeutic compounds. More specifically the present invention pertains to certain fluoro-perhexiline compounds (also referred to herein as FPER compounds) that are useful, for example, in the treatment of disorders (e.g., diseases) including, for example, those which are known to be treated with, or known to be treatable with, perhexiline, including, for example, disorders that are ameliorated by the inhibition of carnitine palmitoyltransferase (CPT); cardiovascular disorders such as: angina pectoris; heart failure (HF); ischaemic heart disease (IHD); cardiomyopathy; cardiac dysrhythmia; stenosis of a heart valve; hypertrophic cardiomyopathy (HCM); coronary heart disease; and other disorders, for example, diabetes and cancer. The present invention also pertains to pharmaceutical compositions comprising such compounds, and the use of such compounds and compositions, for example, in therapy.

### BACKGROUND

A number of publications are cited herein in order to more fully describe and disclose the invention and the state of the art to which the invention pertains. Each of these publications is incorporated herein by reference in its entirety into the present disclosure, to the same extent as if each individual publication was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Perhexiline

Perhexiline, also known as 2-(2,2-dicyclohexylethyl)piperidine, has one chiral centre and two enantiomers, often referred to as (+)-perhexiline and (−)-perhexiline. Apparently, the absolute configurations of (+)-perhexiline and (−)-perhexiline are not yet known.

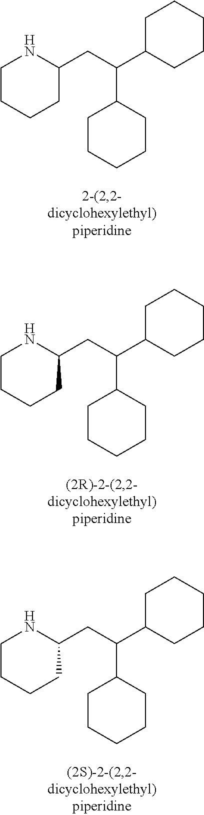 [SMILES: C1CCC(C(CC2CCCCN2)C2CCCCC2)CC1 ; C1CCC(C(C[C@H]2CCCCN2)C2CCCCC2)CC1 ; C1CCC(C(C[C@@H]2CCCCN2)C2CCCCC2)CC1]

Perhexiline is used primarily in Australia and New Zealand as a prophylactic anti-anginal agent. It is typically administered orally, as tablets containing racemic perhexiline, as the maleate salt.

The therapeutic potential of perhexiline has been known for over 40 years (see e.g., Hudak et al., 1970; Cho et al., 1970). It has seen clinical use for the treatment of angina and, more recently, has been investigated for the treatment of heart failure (HF) (see, e.g., Lee et al., 2005), acute coronary syndromes (see, e.g., Willoughby et al., 2002), inoperable aortic stenosis (see, e.g., Unger et al., 1997) and hypertrophic cardiomyopathy (HCM) (see, e.g., Abozguia et al., 2010). On the basis of the latter study, the FDA recently awarded orphan drug status to perhexiline as first line treatment in HCM. It is becoming apparent that altered myocardial energetics plays a prominent role in the pathogenesis of ischaemic heart disease (IHD) and HF; that optimisation of myocardial metabolic efficiency is a highly promising approach to treatment of HF; and that this approach can be additive or synergistic with other therapies (see. e.g., Neubauer, 2007).

By increasing myocardial efficiency, perhexiline has the potential to treat a range of disorders in which impaired cardiac energetics or oxygen deficiency play a role, including, e.g., IHD, hypertrophic cardiomyopathy, congestive HF, aortic stenosis, angina, diabetes, and metabolic syndrome (see, e.g., Ashrafian et al., 2007; Lee et al., 2005).

### Mechanism of Action of Perhexiline

Perhexiline is able to shift myocardial metabolism from fatty acid to carbohydrate utilisation (improved substrate utilisation), thereby enhancing ATP synthesis without affecting oxygen consumption (enhanced cellular energetics). This has been shown to give a functional benefit even for HF patients already receiving optimal drug therapy. The therapeutic action of perhexiline is thought primarily to be due to inhibition of carnitine palmitoyltransferase-1 (CPT-1) and to a lesser extent (CPT-2) (see, e.g., Kennedy et al., 1996; Ashrafian et al., 2007). CPT is an enzyme responsible for mediating mitochondrial uptake of long chain fatty acids, by binding them to carnitine; the acylated carnitine is then taken into the cell by a carnitine acyl-carnitine translocase. Preventing free fatty acid transport across mitochondrial cell membranes causes a corresponding shift to greater carbohydrate usage and an increase in myocardial efficiency (10-40%; more in some studies), in terms of ATP generated per unit of oxygen, as fatty acids consume more oxygen per unit of ATP produced. This oxygen-sparing effect is of particular significance in the treatment of angina and heart failure, allowing cardiac output to remain constant in spite of reduced cardiac oxygen extraction. Perhexiline is both a coronary and systemic vasodilator, giving an increase in coronary and femoral blood flow, in spite of a fall in systemic blood pressure. These effects may also be mediated by the weak L-type calcium channel-blocking effect of perhexiline (see, e.g., Ashrafian et al., 2007); however, at therapeutic levels no systemic vasodilation is evident.

Perhexiline inhibits CPT-1 with modest potency (IC₅₀=77 μM and 148 μM in rat cardiac and liver mitochondria) and inhibits CPT-2 (79 μM) and fatty acid oxidation (8-oxidation) in hepatocytes at 22 μM, resulting in increased lactate and glucose utilisation (see, e.g., Ceccarelli et al., 2011). Perhexiline is the only reversible inhibitor of CPT-1 with proven clinical efficacy and has greater potency against cardiac mitochondrial CPT-1 than hepatic mitochondrial CPT-1 in vitro. Whilst the effective contribution of CPT inhibition has also been questioned (see, e.g., Ceccarelli et al., 2011; Davies et al. 2007b), a further argument in support of the importance of CPT-1 inhibition as the mechanism of action, in spite of the apparent lack of potency, is the tendency for perhexiline to become concentrated in tissues (see, e.g., Ceccarelli et al., 2011), attributed to its amphipathic nature (see, e.g., Ashrafian et al., 2007); thus the concentration in the vicinity of mitochondrial CPT-1 is thought to exceed the modest IC₅₀.

Inhibition of fatty acid metabolism only partially explains the efficacy of perhexiline; other metabolic mechanisms (such as effects on insulin sensitivity), anti-inflammatory activity, and effects on response to nitric oxide, may also play a significant role (see, e.g., Frenneaux, 2002).

There are a number of other drugs that also act as fatty acid oxidation inhibitors and also show utility in the treatment of myocardial diseases; these include etomoxir, amiodarone, trimetazidine, oxfenicine and ranolazine (see, e.g., Ceccarelli et al., 2011).

Notably, the therapeutic utility of perhexiline has been greatly limited by the risk of serious neurotoxicity and hepatotoxicity, caused by a narrow therapeutic index and complex pharmacokinetics. As a consequence, therapeutic use has been highly restricted, primarily to Australia/New Zealand for the treatment of refractory angina pectoris.

Very recently, it has been alleged that “the severe adverse effects of hepatotoxicity and neuropathy observed upon administration of racemic perhexiline are unexpectedly associated with the (+)-enantiomer and not the (−)-enantiomer”. See, e.g., Sallustio et al., 2014.

### Therapeutic Uses of Perhexiline: Heart Failure

Heart failure (HF) arises from the inability of the heart to fill and/or eject blood optimally (see, e.g., Kaye and Krum, 2007). This leads to an increase in the stress placed upon the cardiac wall, neuro-hormonal alterations, left ventricular re-modelling and hypertrophy, and eventual loss of function and inability to maintain the necessary oxygen supply to the body. HF is associated with considerable levels of disability, intolerance towards exertion, fatigue, and breathlessness. HF affects up to 10% of individuals of age 65 years or greater, and has a 50% mortality within 4 years of diagnosis (see, e.g., Kaye and Krum, 2007). HF is most commonly initiated by a heart attack.

The treatment of systolic HF, in which there is depressed left ventricular ejection fraction, is served by a number of different therapeutic approaches, which aim to halt or reverse the progression of myocardial dysfunction. These include: angiotensin-converting enzyme inhibitors, angiotensin II receptor blockers, β-adrenergic receptor antagonists, vasodilators and diuretics, with many other therapeutic approaches under investigation (see, e.g., Tamargo and Lopez-Sendon, 2011). In moderate heart failure, free fatty acid metabolism is increased, resulting in impaired energy generation and increased insulin resistance (see, e.g., Tamargo and Lopez-Sendon, 2011); consequently, metabolic therapy is a highly promising prospect for the treatment of HF. Clinical studies with perhexiline showed encouraging results, including unprecedented improvements in VO_{2 max}, quality of life improvement, and left-ventricular ejection fraction (see, e.g., Lee et al., 2005).

Perhexiline has particularly strong potential in the treatment of diastolic heart failure (HF with preserved ejection fraction; sometimes referred to as HFPEF). This is primarily a disease affecting an elderly, often female, population. Diastolic HF is a disease characterised by left ventricular stiffness and slow left ventricular relaxation. It accounts for up to 50% of cases of HF, with mortality of up to 17% (see, e.g., Sweitzer and Stevenson, 2000). Unlike systolic HF, there are no current effective pharmacotherapies (see, e.g., Tamargo and Lopez-Sendon, 2011). Perhexiline is in Phase II clinical trials for the treatment of diastolic heart failure, for myocardial protection with left ventricular injury.

### Therapeutic Uses of Perhexiline: Ischaemic Heart Disease

Ischaemic heart disease (IHD) is caused by insufficient oxygen reaching the myocardium, and is the main cause of death in western countries, usually by dysrhythmia or from mechanical failure of the ventricle. It manifests clinically as angina or myocardial infarction.

Angina is one of the main symptoms of IHD. It is often a predictable chest pain on exertion, brought on by ischaemia of the heart muscle, caused by obstruction (often due to atherosclerosis) or spasm of the coronary arteries and insufficient oxygen reaching the myocardium. The main treatments for angina are organic nitrates, β-blockers and calcium channel blockers. Anginal pain is caused by anaerobic glycolysis and fatty acid β-oxidation, leading to acidosis and an accumulation of lactate in myocardial tissue. By reduction of fatty acid β-oxidation and optimising oxygen utilisation (thus reducing anaerobic metabolism and production of lactate), perhexiline can reduce anginal pain.

Myocardial infarction (heart attack) is often caused by interruption of the blood supply to the heart by an unstable atherosclerotic plaque becoming detached and occluding the artery or leading to the formation of a blood clot (thrombus) which then blocks the artery. If the blockage does not clear rapidly, then the myocardial tissue can die, leading to permanent damage to the heart and impairment of function. Impairment of function can often lead to cardiac arrest or heart failure. Treatment of myocardial infarction is based on prevention of reoccurrence; therapies include nitrates, ACE inhibitors, β-blockers, aspirin, thrombolytic and anti-platelet drugs, and statins. The pathogenesis of IHD involves a number of factors, including inflammation and activation of immune cells, and impaired platelet and vascular response to nitric oxide, leading to vasoconstriction and thrombogenesis. The ability of perhexiline to sensitise platelets to the anti-aggregatory effects of nitric oxide plays a major role in the resolution of ischaemia (see, e.g., Willoughby et al., 2002).

Perhexiline has been used clinically for the treatment of ischaemic heart disease and angina. Perhexiline was shown to give a greater than 50% reduction in the symptoms of angina, in comparison with nitroglycerin. Perhexiline is also highly effective in the treatment of acute coronary syndromes (see, e.g., Ashrafian et al., 2007; Willoughby et al., 2002). Both the oxygen-sparing and coronary vasodilation activities of perhexiline are expected to play a role in these therapeutic benefits.

### Therapeutic Uses of Perhexiline: Aortic Stenosis

Aortic stenosis is a narrowing of the aortic valve in the heart, restricting blood flow through the valve, forcing the heart to work harder and leading to an oxygen debt. The most common symptoms are breathlessness on exertion; more severe cases may involve chest pain and the risk of sudden death. The main therapy is surgery and valve replacement, but this may involve unacceptable risks in the elderly. Aortic stenosis involves poor platelet responsiveness to nitric oxide and also leads to an oxygen debt. In a clinical trial, it was shown that perhexiline gave clear symptomatic improvement in 13 out of 15 elderly patients with aortic stenosis and who were unsuitable for valve replacement (see, e.g., Unger et al., 1997).

### Therapeutic Uses of Perhexiline: Hypertrophic Cardiomyopathy

Hypertrophic cardiomyopathy (HCM) is the most common inherited cardiac condition and involves thickening of the myocardium and can lead to sudden death. It has been shown that HCM is, to some degree, a disease which involves energy deficiency. As an agent which improves cardiac energetics, perhexiline has been shown to give improvements in VO₂ and left ventricular dysfunction during exercise in patients with symptomatic non-obstructive HCM (see, e.g., Abozguia et al., 2010).

### Therapeutic Uses of Perhexiline: Diabetes

Diabetes is often associated with heart failure; HF is present in 30-40% of cases of diabetes (see, e.g., Tamargo and Lopez-Sendon, 2011). Insulin resistance has been found to increase myocardial uptake of free fatty acids, thus reducing glucose uptake and decreasing cardiac efficiency. By reducing free fatty acid uptake, perhexiline and derivatives have the potential to show therapeutic benefit for diabetes patients (see, e.g., Tamargo and Lopez-Sendon, 2011).

### Therapeutic Uses of Perhexiline: Cancer

The metabolic requirements of proliferating tumour cells are significantly different to those of normal cells, focussing more heavily on the biosynthetic processes required for proliferation. Cancer cells have a number of metabolic solutions which allow the balance between growth and survival to be maintained; in particular, many are found to metabolise glucose by aerobic glycolysis. Cells growing in these conditions tend to proliferate quicker, as glycolysis provides key carbon backbones required for biosynthesis, and may be more able to invade and metastasise. Whilst the mechanisms involved are not entirely clear, there is strong evidence that perturbation of tumour cell metabolism will have the effect of increasing sensitivity to treatment, and represent a promising new therapeutic approach.

Carnitine palmitoyltransferase-1 (CPT-1) has been reported as a metabolic target for cancer therapy (see, e.g., Galluzzi et al., 2013; Vander Heiden, 2011). The inhibition of CPT-1 exerts anticancer effects in vitro and in vivo, most likely by its inhibitory action on fatty acid β-oxidation. Compounds with the same target (CPT-1) as perhexiline have been shown to decrease viability and resistance to chemotherapy of glioblastoma and acute myeloid leukaemia cells (see, e.g., Pike et al., 2011; Samudio et al., 2010; Zaugg et al., 2011). Perhexiline and perhexiline derivatives therefore have potential in anti-neoplastic therapies to target tumour metabolism, either alone or in combination with existing therapeutic regimens (e.g., to increase susceptibility to chemotherapy).

### Perhexiline Derivatives

In spite of the therapeutic potential and the toxicity issues, which led to its removal from clinical usage throughout much of the world, few attempts have been made to modify the chemical structure of perhexiline. To date, derivatives with improved properties (relative to perhexiline) have not been reported.

The synthesis of perhexiline has been described previously (see, e.g., Horgan et al., U.S. Pat. No. 4,191,828; Horgan et al., U.S. Pat. No. 4,069,222). N-Substituted derivatives have also been described (see, e.g., Tassoni et al., 2007). Further derivatives, in which the piperidine has been replaced by other amine-bearing groups have also been described (see, e.g., LeClerc et al., 1982). Derivatives in which the carbon separating the two cyclohexyl groups has been substituted with a hydroxyl group have been reported (see, e.g., Tilford and van Campen, 1954). The synthesis of a deuterated derivative has also been described (see, e.g., Schou, 2010).

Synthesis of derivatives in which the cyclohexyl groups are modified has not been reported. However, it appears that “hydroxyperhexiline” (i.e., 4-[1-(cyclohexyl)-2-(2-piperidinyl)ethyl]cyclohexanol; CAS Registry No 89787-89-3); “trans-hydroxyperhexiline” (i.e., trans-4-[1-(cyclohexyl)-2-(2-piperidinyl)ethyl]cyclohexanol; CAS Registry No 917877-74-8); and “cis-hydroxyperhexiline” (i.e., cis-4-[1-(cyclohexyl)-2-(2-piperidinyl)ethyl]cyclohexanol; CAS Registry No 917877-73-7) are commercially available. Furthermore, as discussed below, 4-monohydroxy metabolites have been identified and isolated by liquid chromatography (see, e.g., Davies et al., 2006).

### Metabolism and Toxicology of Perhexiline

The toxicological issues associated with perhexiline are believed to relate primarily to inter-individual variance in its metabolism.

Metabolism of perhexiline is mediated primarily by cytochrome P450 2D6 (CYP2D6) and phase I metabolism can give one of six possible hydroxy or dihydroxy metabolites. Depending upon whether the subject has zero, one, two, or multiple copies of the gene for CYP2D6 (as well as how well the allelic variant(s) function), a patient can be categorised as a “poor metaboliser” (PM), an “intermediate metaboliser” (IM), an “extensive metaboliser” (EM), or an “ultra-rapid metaboliser” (UM). This polymorphism of CYP2D6 gives substantial inter-individual variation in the rate of metabolism, with an approximate 100-fold difference in perhexiline clearance and required dosage between EM and PM patients. Up to 10% of Caucasians are poor metabolisers.

The predominant metabolite for each enantiomer is the cis-4-axial (cis-OH-PHX), (see, e.g., Ashrafian et al., 2007), but the two trans-metabolites (trans1-OH-PHX and trans2-OH-PHX) are also produced in substantial amounts (see, e.g., Davies et al., 2006). (In the following structures, chiral centres are indicated with an asterisk (*).)

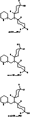

In poor metabolisers, the ability to form cis-OH-PHX is impaired and the primary metabolite is the trans2-OH-PHX. In extensive metabolisers, cis-OH-PHX and trans1-OH-PHX are the major metabolites detected, whilst trans2-OH-PHX is not observed, with the evidence suggesting that this is due to it being a substrate for CYP2D6 (see, e.g., Davies et al., 2006) and thus rapidly removed, rather than not formed. In the absence of CYP2D6, perhexiline can be metabolised by CYP2B6 or CYP3A4, for which it has lower affinity than 2D6 (see, e.g., Ashrafian et al., 2007).

A strong correlation has been made between (a) the incidence of long-term neurotoxicity and hepatotoxicity in patients treated with perhexiline, and (b) patient CYP2D6 status. Patients who showed no toxicological side-effects after treatment had plasma perhexiline to metabolite ratios of 0.3, whilst patients who showed side-effects had ratios of 2.8 (see, e.g., Singlas et al., 1978). Thus, it has been widely accepted that perhexiline toxicity is primarily due to poor metabolism leading to the accumulation of toxic levels of the drug; the toxicity may be related to inhibition of CPT-1, but this has been questioned (see, e.g., Ceccarelli, 2011).

To avoid this risk of toxicity, monitoring of plasma perhexiline concentrations is essential. However, this is inconvenient and precludes wider use of the drug outside of a controlled clinical environment. In principle, phenotyping of patients can be conducted to identify poor metabolisers, who can either be selected for close monitoring, or can be excluded from use of the drug. However, not only is genotyping/phenotyping controversial, but also within each group there is substantial inter-individual variation, and within the EM group a 5-fold difference in the rate of perhexiline metabolism has been reported (see, e.g., Ashrafian et al., 2007). Furthermore, as CYP2D6 is also a substrate for many drugs and is inhibited by a number of common drugs (including fluoxetine), perhexiline metabolism may also be impaired by this mechanism. In addition, perhexiline metabolism may be reduced in patients with impaired liver function. Consequently, the need for therapeutic plasma monitoring cannot safely be excluded.

A further consideration is the activity and fates of metabolites, each of which will be formed in different proportions and cleared at a different rate. These metabolites may be inactive, pharmacologically beneficial, or toxic, and thus may play a major role in the therapeutic/toxicological profile of perhexiline.

It is clear that compounds which have the same therapeutic profile as perhexiline, but which lack the metabolic liability associated with CYP2D6 metabolism or the ability to form potentially-toxic hydroxyl-metabolites, would be highly effective therapeutic agents for the treatment of a range of cardiovascular conditions. In the absence of complex and variable pharmacokinetics, such a compound would not be restricted to limited patient populations or to use in a clinical environment, and thus could benefit a wider patient population for a range of cardiovascular diseases.

### Influence of Chirality on Perhexiline Activity and Toxicology

It has been shown that the two enantiomers of perhexiline have different pharmacokinetic metabolic properties (see, e.g., Sallustio et al., 2014). However, differences in their pharmacological properties have not yet been reported.

Significant enantioselectivity has been demonstrated in the oxidative metabolism of perhexiline (see, e.g., Davies et al., 2007a; Inglis et al., 2007). It has been shown that CYP2D6 accounts for more than 90% of the metabolism of both (+)- and (−)-perhexiline. CYP2D6 shows significant selectivity for (−)-perhexiline (see, e.g., Inglis et al., 2007). CYP2D6 metabolism gives only the cis-OH-metabolite from (−)-perhexiline, but both cis- and trans-OH-metabolites from (+)-perhexiline. In “poor metabolisers” (PM), the enzymes metabolising (+)- and (−)-perhexiline are CYP3A4 and CYP2B6, forming only trans-OH-metabolites.

The (−)-enantiomer is metabolised more rapidly in vitro by human liver microsomes and cleared more rapidly in patients, resulting in a greater systemic exposure to (+)-perhexiline. One study reported plasma area under the curve (AUC) values that were 2.5-fold greater for (+)-perhexiline, and 28-fold greater for cis-OH-perhexiline following administration of (−)-perhexiline compared with (+)-perhexiline (see, e.g., Inglis et al., 2007), demonstrating the more rapid removal of the parent compound and the more rapid formation/slower elimination of the major metabolite for (−)-perhexiline.

Furthermore the ratio of systemic exposure of (+)- to (−)-perhexiline in “extensive metabolisers” (EM) is 1.5, compared to 2.3 in “poor metabolisers” (PM) (see, e.g., Davies et al., 2007b). This demonstrates that non-CYP2D6 mediated metabolic processes also show significant enantioselectivity, which may be mediated by enantioselective biliary or intestinal excretion (see, e.g., Davies et al., 2007b). Thus, the relative exposure of the (+)-enantiomer is significantly higher in “poor metabolisers” (PM) (see, e.g., Inglis et al., 2007), potentially further increasing the risk of clinical toxicity.

Drugs often interact with chiral biological targets. In common with many chiral compounds, the enantiomers of perhexiline are expected to show different pharmacological properties, giving different therapeutic and toxicological effects. The putative target for perhexiline is CPT-1, a transporter protein which exists in three different isoforms: CPT-1A, expressed in the liver; CPT-1B, expressed in adult cardiomyocytes; and CTP-1C, expressed in the central nervous system. The two enantiomers are likely to have different selectivity between these three isoforms and thus different toxicology (inhibition of the liver isoforms) and efficacy (inhibition of the cardiac isoforms). Furthermore, each of the enantiomers, and the other minor metabolites, will then be cleared at a different rate.

### Fluorinated Perhexiline

In an effort to address the above problems, the Inventors have identified the fluoro-perhexiline (FPER) compounds described herein.

Without wishing to be bound to any particular theory, the Inventors believe that the FPER compounds described herein have been protected against the major route of metabolism that acts upon perhexiline (specifically, CYP2D6-mediated oxidation of the 4-position of one or both cyclohexyl groups, to give an alcohol) by the replacement of the hydrogen with a fluorine. In addition to preventing CYP2D6-mediated metabolism (which is regarded as one of the major drawbacks of perhexiline, and which prevents wider clinical use), these modifications have also increased metabolic stability as a whole and have not led to the introduction of other CYP-mediated routes of oxidative metabolism.

If a drug is to show oral activity, the drug must be sufficiently resistant to first-pass metabolism by metabolic enzymes contained within the liver so as to be able to enter the circulation and permit sufficient quantities to reach the biological target. The optimization of pharmacokinetic properties (action of the body on the drug) of a drug is a developmental barrier of equal challenge as compared to the optimization of pharmacodynamic properties (action of the drug on the body).

By improving general metabolic stability and removing the susceptibility to CYP2D6-mediated metabolism (and the highly-unfavourable complications this entails), the FPER compounds described herein have substantially improved properties as oral therapeutic agents, as compared to perhexiline, and thus may find widespread use in therapy, for example, in the treatment of the diseases in which perhexiline has shown clinical and experimental efficacy.

### SUMMARY OF THE INVENTION

One aspect of the invention pertains to certain fluoro-perhexiline compounds (also referred to herein as FPER compounds), as described herein.

Another aspect of the invention pertains to a composition (e.g., a pharmaceutical composition) comprising an FPER compound, as described herein, and a pharmaceutically acceptable carrier or diluent.

Another aspect of the invention pertains to a method of preparing a composition (e.g., a pharmaceutical composition) comprising the step of mixing an FPER compound, as described herein, and a pharmaceutically acceptable carrier or diluent.

Another aspect of the present invention pertains to a method of inhibiting carnitine palmitoyltransferase (CPT) (e.g., CPT-1, CPT-1A, CPT-1B, CPT-1C, CPT-2), in vitro or in vivo, comprising contacting the CPT with an effective amount of an FPER compound, as described herein.

Another aspect of the present invention pertains to a method of inhibiting carnitine palmitoyltransferase (CPT) (e.g., CPT-1, CPT-1A, CPT-1B, CPT-1C, CPT-2) in a cell, in vitro or in vivo, comprising contacting the cell with an effective amount of an FPER compound, as described herein.

Another aspect of the present invention pertains to an FPER compound, as described herein, for use in a method of treatment of the human or animal body by therapy, for example, for use a method of treatment of a disorder (e.g., a disease) as described herein.

Another aspect of the present invention pertains to use of an FPER compound, as described herein, in the manufacture of a medicament for treatment, for example, treatment of a disorder (e.g., a disease) as described herein.

Another aspect of the present invention pertains to a method of treatment, for example, of a disorder (e.g., a disease) as described herein, comprising administering to a patient in need of treatment a therapeutically effective amount of an FPER compound, as described herein, preferably in the form of a pharmaceutical composition.

In one embodiment, the treatment is treatment of a disorder (e.g., a disease) that is ameliorated by the inhibition of carnitine palmitoyltransferase (CPT).

In one embodiment, the treatment is treatment of a disorder (e.g., a disease) that is ameliorated by inhibition of fatty acid oxidation (e.g., β-oxidation).

In one embodiment, the treatment is treatment of a disorder (e.g., a disease) that is characterised by impaired cardiac energetics.

In one embodiment, the treatment is treatment of a disorder (e.g., a disease) that is characterised by oxygen deficiency.

In one embodiment, the treatment is treatment of ischaemia.

In one embodiment, the treatment is treatment of a cardiovascular disorder (e.g., a cardiovascular disease).

In one embodiment, the treatment is treatment of angina pectoris; heart failure (HF); left or right ventricular failure; pulmonary heart disease; ischaemic heart disease (IHD); cardiomyopathy; cardiac dysrhythmia; stenosis of a heart valve; hypertrophic cardiomyopathy (HCM); or coronary heart disease.

In one embodiment, the treatment is treatment of angina pectoris (also known as angina), for example, angina pectoris caused by coronary heart disease; angina pectoris caused by ischaemia; severe angina pectoris; or unresponsive or refractory angina pectoris.

In one embodiment, the treatment is treatment of heart failure (HF), for example, heart failure caused by ischaemia; congestive heart failure; chronic heart failure; moderate heart failure; systolic heart failure; diastolic heart failure; or diastolic heart failure with left ventricular injury.

In one embodiment, the treatment is treatment of left or right ventricular failure, for example, of various etiologies.

In one embodiment, the treatment is treatment of pulmonary heart disease, for example, pulmonary heart disease caused by pulmonary hypertension; pulmonary heart disease caused by chronic obstructive lung disease; or pulmonary heart disease caused by emphysema.

In one embodiment, the treatment is treatment of ischaemic heart disease (IHD), for example, ischaemic heart disease caused by coronary heart disease; ischaemic heart disease caused by obstruction of the coronary artery; ischaemic heart disease caused by spasm of the coronary artery; severe ischaemic heart disease (e.g., in a patient awaiting coronary revascularisation); or refractory ischaemic heart disease (e.g., in a patient with ischaemic symptoms refractory to other therapeutic measures).

In one embodiment, the treatment is treatment of cardiomyopathy, including, for example, cardiomyopathy due to ischaemic heart disease; or cardiomyopathy due to hypertension.

In one embodiment, the treatment is treatment of cardiac dysrhythmia (also known as cardiac arrhythmia or irregular heartbeat), for example, cardiac dysrhythmia caused by ischaemia.

In one embodiment, the treatment is treatment of stenosis of a heart valve, for example, aortic stenosis, for example, inoperable aortic stenosis.

In one embodiment, the treatment is treatment of hypertrophic cardiomyopathy (HCM), for example, symptomatic non-obstructive hypertrophic cardiomyopathy.

In one embodiment, the treatment is treatment of coronary heart disease.

In one embodiment, the treatment is treatment of a metabolic-related disorder, for example, diabetes (e.g., type 1 diabetes, type 2 diabetes, diabetes associated with heart failure); hyperglycemia; hyperlipidemia; hypertriglyceridemia; dyslipidemia; syndrome X (also known as metabolic syndrome); or obesity.

In one embodiment, the treatment is treatment of cancer, e.g., acute myeloid leukaemia; adrenal gland cancer; biliary tract cancer; bladder cancer; bone cancer; bowel cancer; brain cancer; breast cancer; colon cancer; colorectal cancer; endometrial cancer; gastrointestinal cancer; genito-urinary cancer; glioma; glioblastoma; gynaecological cancer; head cancer; Hodgkin's disease; Kaposi's sarcoma; kidney cancer; large bowel cancer; leukaemia; liver cancer; lung cancer; lymphoma; lymphocytic leukaemia (lymphoblastic leukaemia); malignant melanoma; mediastinum cancer; melanoma; myeloma; myelogenous leukaemia (myeloid leukaemia); nasopharyngeal cancer; neck cancer; nervous system cancer; non-Hodgkin's lymphoma; non-small cell lung cancer; oesophagus cancer; osteosarcoma; ovarian cancer; pancreatic cancer; prostate cancer; rectal cancer; renal cell carcinoma; sarcoma; skin cancer; small bowel cancer; small cell lung cancer; soft tissue sarcoma; squamous cancer; stomach cancer; testicular cancer; or thyroid cancer.

Another aspect of the present invention pertains to a kit comprising (a) an FPER compound, as described herein, preferably provided as a pharmaceutical composition and in a suitable container and/or with suitable packaging; and (b) instructions for use, for example, written instructions on how to administer the compound.

Another aspect of the present invention pertains to an FPER compound obtainable by a method of synthesis as described herein, or a method comprising a method of synthesis as described herein.

Another aspect of the present invention pertains to an FPER compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION OF THE INVENTION

### Compounds

One aspect of the present invention relates to certain compounds which are related to 2-(2,2-dicyclohexylethyl)piperidine, also known as “perhexiline”.

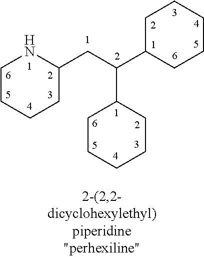 [SMILES: C1CCC(C(CC2CCCCN2)C2CCCCC2)CC1]

All of the compounds of the present invention have one or two fluoro groups at the para-position of one or both of the cyclohexyl groups of perhexiline.

Thus, one aspect of the present invention is a compound of the following formula, or a pharmaceutically acceptable salt, hydrate, or solvate thereof, wherein —R^{1A}, —R^{1B}, —R^{2A}, —R^{2B}, and —R^{N1} are as defined herein (for convenience, collectively referred to herein as “fluoro-perhexiline compounds” and “FPER compounds”):

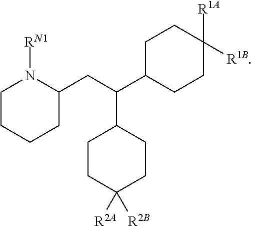

Some embodiments of the invention include the following:

(1) A compound of the following formula:

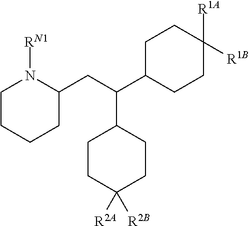

or a pharmaceutically acceptable salt, hydrate, or solvate thereof;
wherein:

—R^{1A} is independently —H or —F;

—R^{1B} is independently —H or —F;

—R^{2A} is independently —H or —F;

—R^{2B} is independently —H or —F;

with the proviso that at least one of R^{1B}, —R^{2A}, and —R^{2B} is —F;

and wherein:
—R^{N1} is independently —H, —R^{NN}, or —C(═O)—R^{NNN};

—R^{NN} is saturated linear or branched C₁₋₄alkyl.
—R^{NNN} is independently saturated linear or branched C₁₋₄alkyl, phenyl, or benzyl.

Note that the compounds have at least one chiral centre, specifically, the carbon ring atom adjacent the ring nitrogen atom, to which the 2,2-dicyclohexylethyl group is attached, marked with an asterisk (*) in the following formula. Unless otherwise stated, the carbon atom at this position may be in either (R) or (S) configuration.

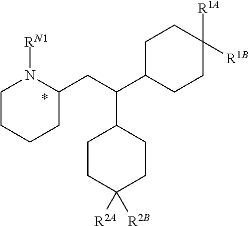

Also note that, depending upon the identity of the groups —R^{1A}, —R^{1B}, —R^{2A}, and —R^{2B}, the compounds may have a second chiral centre, specifically, the carbon atom to which the two cyclohexyl groups are attached, marked with an asterisk (*) in the following formula.

Unless otherwise stated, the carbon atom at this position may be in either (R) or (S) configuration.

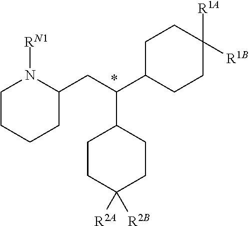

Also note that when one of —R^{1A} and —R^{1B} is —F, and the other is —H, the —F group may be positioned “cis” or “trans” with respect to the rest of the molecule (that is, on the cyclohexyl ring to which it is attached, with respect to the rest of the compound at the para position). Similarly, when one of —R^{2A} and —R^{2B} is —F, and the other is —H, the —F group may be positioned “cis” or “trans” (that is, on the cyclohexyl ring to which it is attached, with respect to the rest of the compound at the para position).

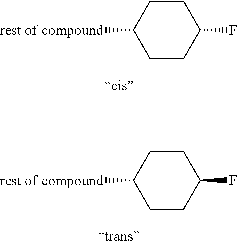

Further note that the cyclohexyl rings are expected to take the preferred “chair” conformation. As a consequence, when one of —R^{1A} and —R^{1B} is —F, and the other is —H, the —F group may be positioned “axially” or “equatorially” (that is, on the cyclohexyl ring). Similarly, when one of —R^{2A} and —R^{2B} is —F, and the other is —H, the —F group may be positioned “axially” or “equatorially” (that is, on the cyclohexyl ring).

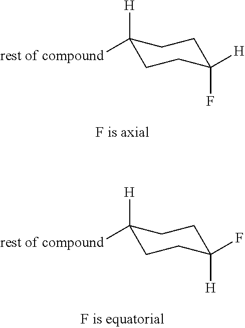

(2) A compound according to (1), wherein —R^{1A} is —F.

(3) A compound according to (1), wherein —R^{1A} is —H.

(4) A compound according to any one of (1) to (3), wherein —R^{1B} is —H.

(5) A compound according to any one of (1) to (3), wherein —R^{1B} is —F.

(6) A compound according to any one of (1) to (5), wherein —R^{2A} is —H.

(7) A compound according to any one of (1) to (5), wherein —R^{2A} is —F.

(8) A compound according to any one of (1) to (7), wherein —R^{2B} is —H.

(9) A compound according to any one of (1) to (7), wherein —R^{2B} is —F.

(10) A compound according to any one of (1) to (9), which is a compound of the following formula, or a pharmaceutically acceptable salt, hydrate, or solvate thereof:

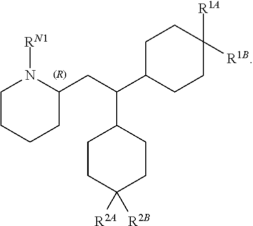

(11) A compound according to any one of (1) to (9), which is a compound of the following formula, or a pharmaceutically acceptable salt, hydrate, or solvate thereof:

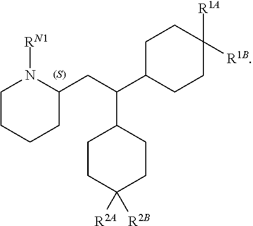

(12) A compound according to (1), which is a compound of the following formula, or a pharmaceutically acceptable salt, hydrate, or solvate thereof:

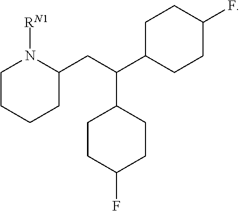

(13) A compound according to (1), which is a compound of the following formula, or a pharmaceutically acceptable salt, hydrate, or solvate thereof:

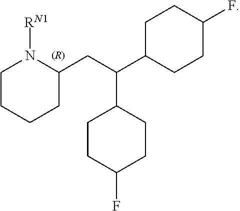

(14) A compound according to (1), which is a compound of the following formula, or a pharmaceutically acceptable salt, hydrate, or solvate thereof:

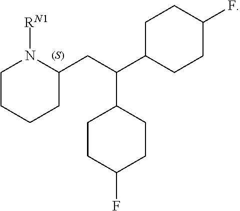

(15) A compound according to (1), which is a compound of the following formula, or a pharmaceutically acceptable salt, hydrate, or solvate thereof:

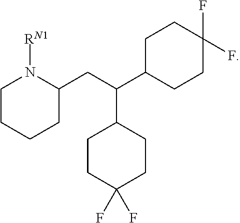

(16) A compound according to (1), which is a compound of the following formula, or a pharmaceutically acceptable salt, hydrate, or solvate thereof:

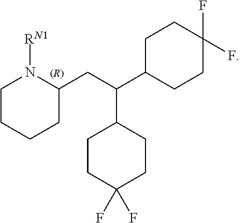

(17) A compound according to (1), which is a compound of the following formula, or a pharmaceutically acceptable salt, hydrate, or solvate thereof:

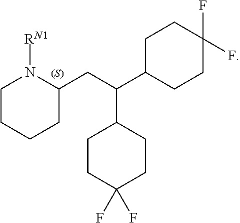

(18) A compound according to (1), which is a compound of the following formula, or a pharmaceutically acceptable salt, hydrate, or solvate thereof:

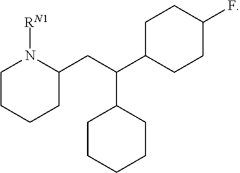

(19) A compound according to (1), which is a compound of the following formula, or a pharmaceutically acceptable salt, hydrate, or solvate thereof:

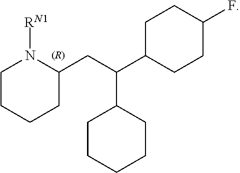

(20) A compound according to (1), which is a compound of the following formula, or a pharmaceutically acceptable salt, hydrate, or solvate thereof:

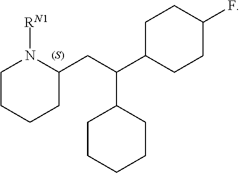

(21) A compound according to (1), which is a compound of the following formula, or a pharmaceutically acceptable salt, hydrate, or solvate thereof:

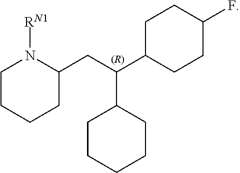

(22) A compound according to (1), which is a compound of the following formula, or a pharmaceutically acceptable salt, hydrate, or solvate thereof:

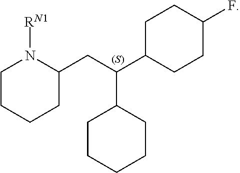

(23) A compound according to (1), which is a compound of the following formula, or a pharmaceutically acceptable salt, hydrate, or solvate thereof:

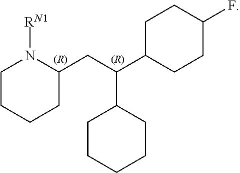

(24) A compound according to (1), which is a compound of the following formula, or a pharmaceutically acceptable salt, hydrate, or solvate thereof:

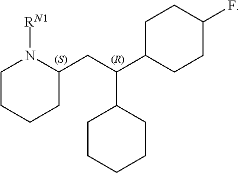

(25) A compound according to (1), which is a compound of the following formula, or a pharmaceutically acceptable salt, hydrate, or solvate thereof:

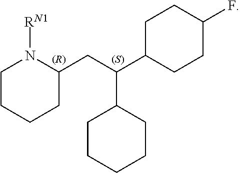

(26) A compound according to (1), which is a compound of the following formula, or a pharmaceutically acceptable salt, hydrate, or solvate thereof:

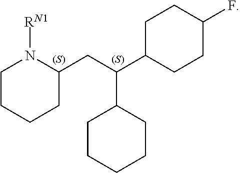

(27) A compound according to (1), which is a compound of the following formula, or a pharmaceutically acceptable salt, hydrate, or solvate thereof:

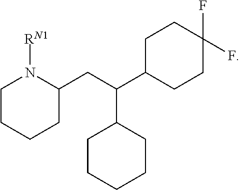

(28) A compound according to (1), which is a compound of the following formula, or a pharmaceutically acceptable salt, hydrate, or solvate thereof:

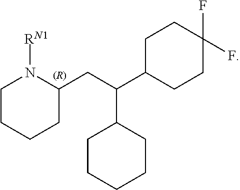

(29) A compound according to (1), which is a compound of the following formula, or a pharmaceutically acceptable salt, hydrate, or solvate thereof:

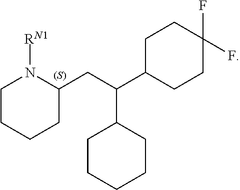

(30) A compound according to (1), which is a compound of the following formula, or a pharmaceutically acceptable salt, hydrate, or solvate thereof:

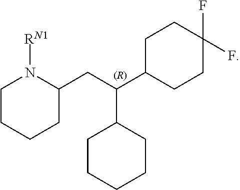

(31) A compound according to (1), which is a compound of the following formula, or a pharmaceutically acceptable salt, hydrate, or solvate thereof:

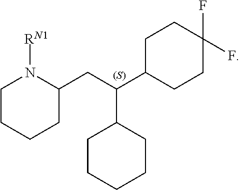

(32) A compound according to (1), which is a compound of the following formula, or a pharmaceutically acceptable salt, hydrate, or solvate thereof:

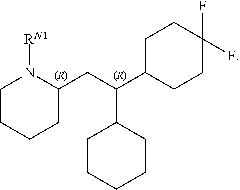

(33) A compound according to (1), which is a compound of the following formula, or a pharmaceutically acceptable salt, hydrate, or solvate thereof:

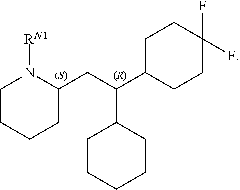

(34) A compound according to (1), which is a compound of the following formula, or a pharmaceutically acceptable salt, hydrate, or solvate thereof:

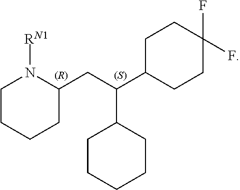

(35) A compound according to (1), which is a compound of the following formula, or a pharmaceutically acceptable salt, hydrate, or solvate thereof:

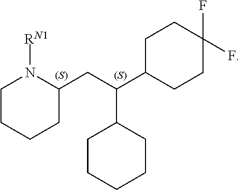

(36) A compound according to any one of (1) to (35), wherein, if: one of —R^{1A} and —R^{1B} is —F, and the other is —H, then: the —F group is positioned cis.

(37) A compound according to any one of (1) to (35), wherein, if: one of —R^{1A} and —R^{1B} is —F, and the other is —H, then: the —F group is positioned trans.

(38) A compound according to any one of (1) to (37), wherein, if: one of —R^{2A} and —R^{2B} is —F, and the other is —H, then: the —F group is positioned cis.

(39) A compound according to any one of (1) to (37), wherein, if: one of —R^{2A} and —R^{2B} is —F, and the other is —H, then: the —F group is positioned trans.

(40) A compound according to any one of (1) to (39), wherein, if: one of —R^{1A} and —R^{1B} is —F, and the other is —H, then: the —F group is positioned axially.

(41) A compound according to any one of (1) to (39), wherein, if: one of —R^{1A} and —R^{1B} is —F, and the other is —H, then: the —F group is positioned equatorially.

(42) A compound according to any one of (1) to (41), wherein, if: one of —R^{2A} and —R^{2B} is —F, and the other is —H, then: the —F group is positioned axially.

(43) A compound according to any one of (1) to (41), wherein, if: one of —R^{2A} and —R^{2B} is —F, and the other is —H, then: the —F group is positioned equatorially.

(44) A compound according to any one of (1) to (43), wherein —R^{N1} is independently —H or —R^{NN}.

(45) A compound according to any one of (1) to (43), wherein —R^{N1} is —H.

(46) A compound according to any one of (1) to (45), wherein —R^{NN}, if present, is independently -Me, -Et, -nPr, -iPr, -nBu, -iBu, or -tBu.

(47) A compound according to any one of (1) to (45), wherein —R^{NN}, if present, is independently -Me, -Et, -nPr, or -iPr.

(48) A compound according to any one of (1) to (45), wherein —R^{NN}, if present, is independently -Me or -Et.

(49) A compound according to any one of (1) to (45), wherein —R^{NN}, if present, is independently -Me.

(50) A compound according to any one of (1) to (49), wherein —R^{NNN}, if present, is saturated linear or branched C₁₋₄alkyl.

(51) A compound according to any one of (1) to (49), wherein —R^{NNN}, if present, is independently -Me, -Et, -nPr, -iPr, -nBu, -iBu, or -tBu.

(52) A compound according to any one of (1) to (49), wherein —R^{NNN}, if present, is independently -Me, -Et, -nPr, or -iPr.

(53) A compound according to any one of (1) to (49), wherein —R^{NNN}, if present, is independently -Me or -Et.

(54) A compound according to any one of (1) to (49), wherein —R^{NNN}, if present, is independently -Me.

(55) A compound according to (1), which is selected from compounds of the following formulae, and pharmaceutically acceptable salts, hydrates, and solvates thereof:

| Code | Synthesis | Compound |
|---|---|---|
| FPER-001 | 8 | 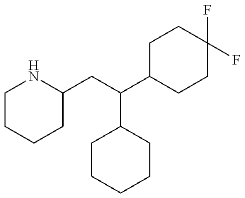 [SMILES: FC1(F)CCC(C(CC2CCCCN2)C2CCCCC2)CC1] |
| FPER-002 | 22 | 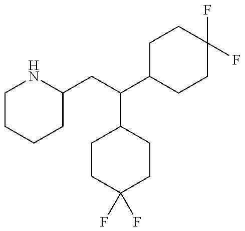 [SMILES: FC1(F)CCC(C(CC2CCCCN2)C2CCC(F)(F)CC2)CC1] |
| FPER-003 | 32 | 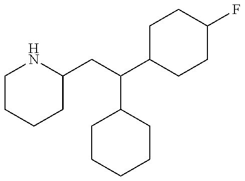 [SMILES: FC1CCC(C(CC2CCCCN2)C2CCCCC2)CC1] |
| FPER-004 | 41 | 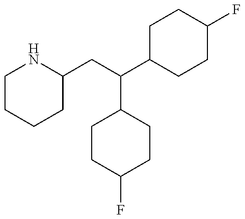 [SMILES: FC1CCC(C(CC2CCCCN2)C2CCC(F)CC2)CC1] |

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables (e.g., —R^{1A}, —R^{1B}, —R^{2A}, —R^{2B}, —R^{N1}, —R^{NN}, —R^{NNN}, etc.) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### Substantially Purified Forms

One aspect of the present invention pertains to FPER compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer(s) or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diastereoisomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

A reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl). However, reference to a specific group or substitution pattern is not intended to include other structural (or constitutional isomers) which differ with respect to the connections between atoms rather than by positions in space. For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl.

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

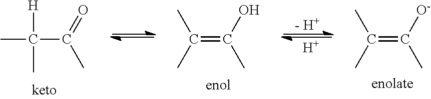

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Examples of some preferred salts suitable for amines (such as the FPER compounds described herein) include: chloride, sulfate, bromide, mesylate, maleate, citrate, tartrate, phosphate, acetate, and iodide. An especially preferred salt may be the maleate salt (since perhexiline itself it currently used in therapy as the maleate salt).

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH—Psec); or, in suitable cases (e.g., cyclic amines), as a nitroxide radical (>N—O.).

### Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

### Chemical Synthesis

Methods for the chemical synthesis of FPER compounds are described herein. These and/or other well-known methods may be modified and/or adapted in known ways in order to facilitate the synthesis of additional FPER compounds described herein.

In general, fluorine is incorporated into compounds using commercially-available building-blocks, most especially aryl fluorides. Where these are not available, nucleophilic fluorinating agents include DAST ((diethylamido)sulfur trifluoride), which reacts with ketones and aldehydes to give difluoroalkyl compounds, and alcohols to give monofluoroalkyls. Electrophilic fluorinating agents usually contain an N—F bond and include Selectfluor® [(1-chloromethyl-4-fluoro-1,4-diazoniabicyclo[2.2.2]octane bis(tetrafluoroborate)], which will react with a number of starting materials, including carbanions generated from carbonyl compounds (see, e.g., Hagmann, 2008).

However, these reactions often require harsh conditions and thus may have limited utility for many chemical structures. For example, a simple route to fluorinated perhexilines would be preparation of the known 4-hydroxycyclohexyl derivative or its toluenesulfonate derivative, and fluoro-dehydroxylation with DAST or bis(2-methoxyethyl)aminosulfur trifluoride (Deoxo-Fluor®). However, in the Inventors' hands, these methods led to elimination rather than fluorination, resulting in cyclohexene formation. Another simple route to fluorinated perhexilines would be use of a commercially-available reagent containing a 4-fluorophenyl group (e.g., 4,4′-difluorobenzophenone) as a starting material with subsequent hydrogenation to give a 4-fluorocyclohexyl moiety. However, in the Inventors' hands, this again led to loss of the fluorine via hydrogenolysis of the C—F bond.

Following an extensive research program, the Inventors have identified and demonstrated suitable methods for the preparation of the FPER compounds.

In one approach, a geminal-difluoro perhexiline derivative can be prepared by the following procedure.

A cyclohexene-carbaldehyde is prepared from an appropriately-protected tosyl-hydrazone-cyclohexanol derivative. The aldehyde is reacted with a cyclohexane-based Grignard agent to give a cyclohexyl-cyclohexenol-methanol. The methyl alcohol is protected, and the cyclohexene reduced, e.g., by hydrogenation, to give the dicyclohexyl skeleton. The protecting group from the cyclohexanol is removed to give the free alcohol, which can then be oxidised to provide the cyclohexanone moiety required for fluorination. Fluorination gives a mixture of the difluoro and vinyl fluoride products which cannot be separated; epoxidation gives a mixture of the difluoro and fluoroepoxide. Removal of the protecting group gives the free methyl alcohol, which can then be oxidised to give a dicyclohexylmethanone, which may then be coupled with a methylpyridine, giving a mixture of positional isomers which cannot be separated, but give the same desired difluorinated perhexiline on hydrogenation.

For example, in one approach, the tosyl-hydrazone-cyclohexanol has been protected with a tetrabutylsilane group and the aldehyde is formed by deprotonation with butyllithium and reaction with dimethylformamide and subsequent hydrolysis. The cyclohexyl-cyclohexenol-methanol is reacted with cyclohexane magnesium chloride in dry tetrahydrofuran, the alcohol protected by benzoylation with benzoyl chloride in dichloromethane, in the presence of dimethylaminopyridine and pyridine, hydrogenated in the presence of 10% palladium on carbon in methanol. The silane protecting group can be removed under acidic conditions, e.g., 1 M hydrochloric acid in methanol/dichloromethane and the ketone generated from the resultant alcohol by oxidation with Dess-Martin periodinane in dichloromethane. The ketone is then fluorinated using diethylaminosulfur trifluoride in dichloromethane, and the inseparable vinyl fluoride impurity oxidised to the fluoroepoxide using meta-chloroperbenzoic acid to give the mixture of difluoro and fluoroepoxides and the benzoyl protecting group removed under basic conditions, e.g., 10% potassium hydroxide in tetrahydrofuran/methanol, to give the geminal difluoro-dicyclohexylmethanol, which can now be separated from the products resulting from hydrolysis of the fluoroepoxide. The ketone is generated from the alcohol by oxidation with Dess-Martin periodinane in dichloromethane and then reacted with 2-methylpyridine to give the mixture of positional isomers, which can then be hydrogenated without separation to give the desired geminal-difluoroperhexiline.

An example of such a method is shown in the following scheme.

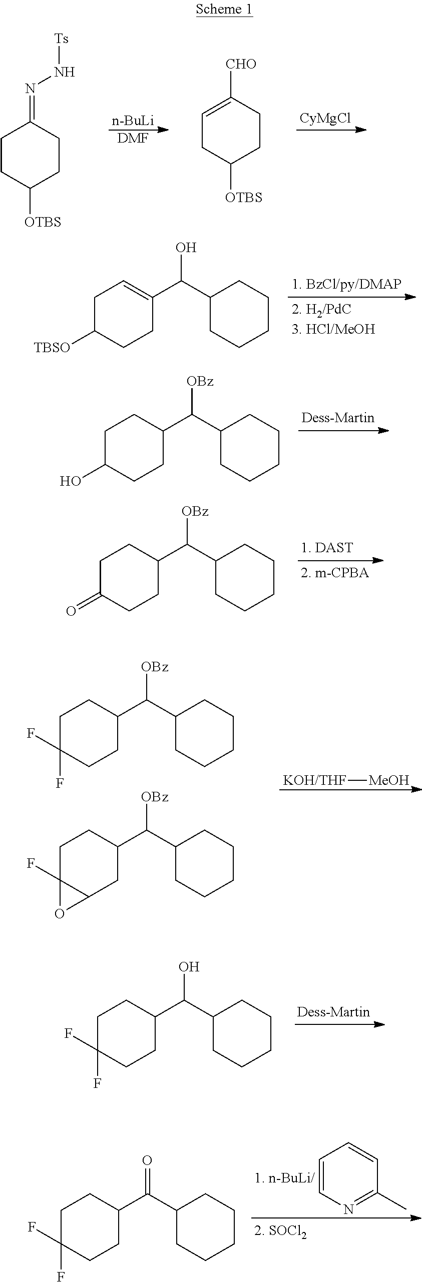

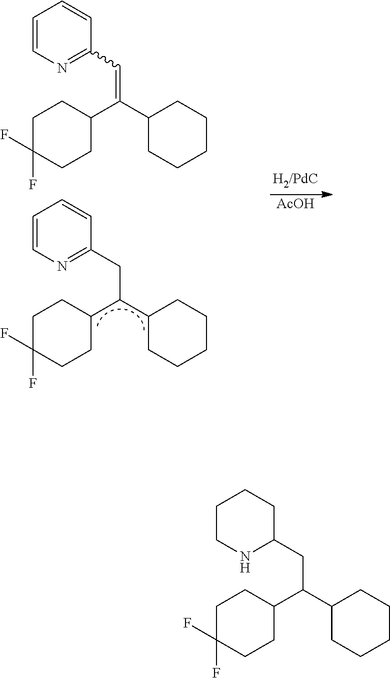

In another approach, a bis geminal-difluoro perhexiline derivative can be prepared by the following procedure.

A (4,4-difluorocyclohexyl)methanol is prepared from an appropriately-protected cyclohexanone derivative. The alcohol is oxidised to the corresponding aldehyde and then reacted with a suitably-protected hydrazone derived from cyclohexanone, to give the difluorocyclohexyl-cyclohexene-methanol. The methyl alcohol is protected, and the cyclohexene deprotected to provide the cyclohexanone moiety required for fluorination. Fluorination gives a mixture of the difluoro and vinyl fluoride products which cannot be separated; epoxidation gives a mixture of the difluoro and fluoroepoxide. Removal of the protecting group gives the free methyl alcohol, which can then be oxidised to give a dicyclohexylmethanone, which may then be coupled with a methylpyridine, giving a mixture of positional isomers which cannot be separated, but give the same desired difluorinated perhexiline on hydrogenation.

For example, in one approach, the protected cyclohexanone derivative is ethyl 4-oxocyclohexanecarboxylate, prepared from ethyl 4-hydroxycyclohexanecarboxylate by oxidation with pyridinium chlorochromate. The ketone is then fluorinated using diethylaminosulfur trifluoride in dichloromethane, the inseparable vinyl fluoride impurity oxidised to the fluoroepoxide using meta-chloroperbenzoic acid to give the mixture of difluoro and fluoroepoxides. The ethyl ester can be reduced to give the methyl alcohol, e.g., using lithium aluminium hydride in THF, and the epoxide hydrolysed with e.g., sodium hydroxide in tetrahydrofuran/methanol, to give a product from which the desired geminal difluoro-dicyclohexylmethanol can now be separated. The geminal difluoro-dicyclohexylmethanol can then be oxidised to the desired aldehyde, e.g., with oxalyl chloride and triethylamine in DCM, with subsequent quenching with water.

In one approach, the hydrazone is 2,4,6-triisopropyl-N-(1,4-dioxaspiro[4.5]decan-8-ylidene)benzenesulfonohydrazide (see, e.g., Hu et al., 2006), which is deprotonated, e.g., with n-butyllithium and reacted with 4,4-difluorocyclohexanecarbaldehyde to give a protected cyclohexyl-cyclohexene-methanol. The secondary alcohol can then also be protected, e.g., as a benzyl alcohol, by reaction with benzoyl chloride in DCM, in the presence of pyridine. The cyclohexenyl moiety can then be hydrogenated and deprotected, and transformed into the required cyclohexanone, e.g., by hydrogenation of the alkene in the presence of Pd/C and subsequent acid hydrolysis, e.g., with HCl in THF.

In one approach, the ketone can then be fluorinated using diethylaminosulfur trifluoride in dichloromethane, again giving the inseparable vinyl fluoride impurity, which is oxidised to the fluoroepoxide using meta-chloroperbenzoic acid to give the mixture of difluoro and fluoroepoxides and the benzoyl protecting group removed under basic conditions, e.g., 10% potassium hydroxide in tetrahydrofuran/methanol, to give the germinal difluoro-dicyclohexylmethanol, which can now be separated from the products resulting from hydrolysis of the fluoroepoxide by column chromatography. The ketone is generated from the alcohol by oxidation with Dess-Martin periodinane in dichloromethane and then reacted with 2-methylpyridine to give the mixture of positional isomers, which can then be hydrogenated without separation to give the desired bis geminal-difluoroperhexiline.

An example of such a method is shown in the following scheme.

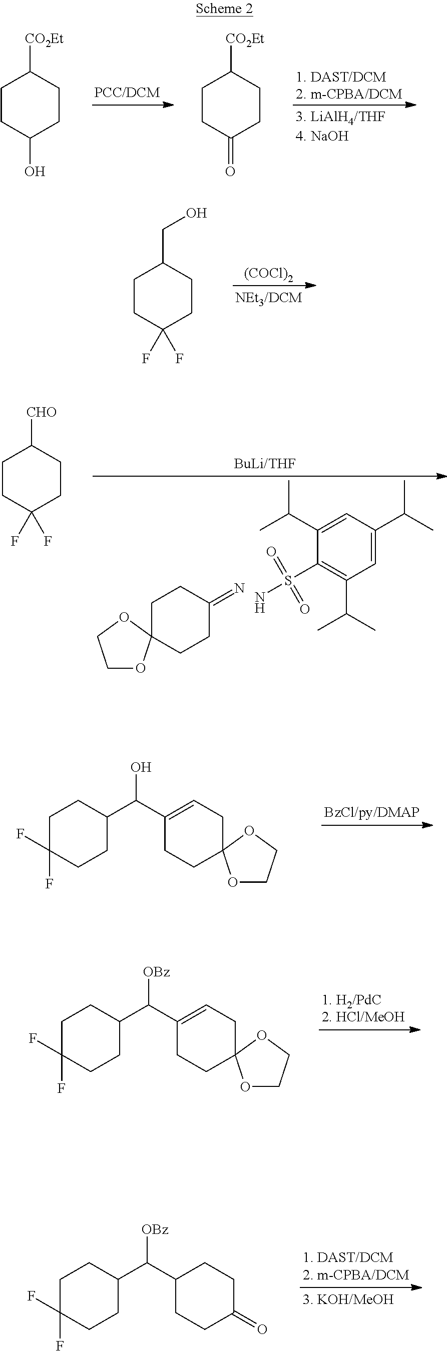

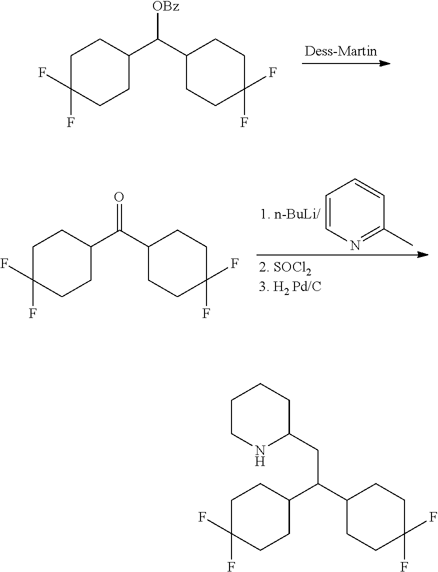

In another approach, a mono-fluoroperhexiline can be prepared via a suitable mono-fluorocyclohexene intermediate. Fluorination of the cyclohexanone with, e.g., DAST will give a mixture of difluorination and elimination, leading to the mono-fluorocyclohexenyl derivative. Under certain conditions, including higher temperatures and use of alternative agents such as 2,2-difluoro-1,3-dimethylimidazolidine (DFI) (see, e.g., Fukumura et al., 2003), the latter process predominates; thus if the reaction is carried out at 50° C. using DAST in DCM, or using DFI in toluene, then the desired ethyl 4-fluorocyclohex-3-enecarboxylate can be prepared from commercially-available ethyl 4-oxocyclohexanecarboxylate. The fluorocyclohexene intermediate can then be further reacted using the methodology described in Scheme 1 to give a mono-fluoroperhexiline.

An example of such a method is shown in the following scheme.

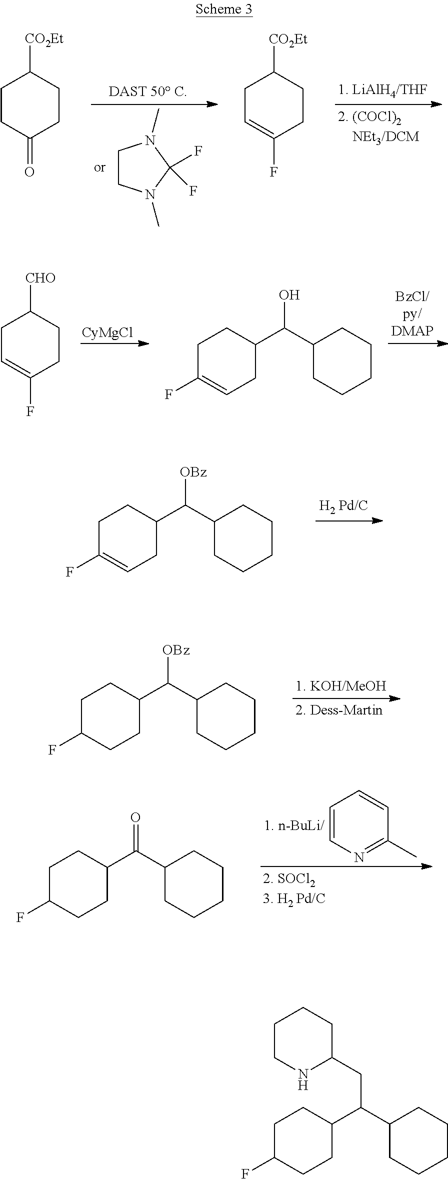

In another approach, the desired mono-fluoroperhexiline intermediate, ethyl 4-fluorocyclohex-3-enecarboxylate, can be prepared can be prepared from ethyl 4-oxocyclohexanecarboxylate via fluorination of the organotin intermediate, itself prepared from the ethyl 4-(trifluoromethylsulfonyloxy)cyclohex-3-enecarboxylate intermediate. The fluorocyclohexene derivative can then be reacted further to give the desired mono-fluoroperhexilineby the methodology described in Scheme 1.

For example, in one approach, ethyl 4-oxocyclohexanecarboxylate is reacted with N-phenyl-bis(trifluoromethanesulfonimide) in the presence of a strong base, for example lithium diisopropylamide (LDA), at −78° C., to give the ethyl 4-(trifluoromethylsulfonyloxy) cyclohex-3-enecarboxylate intermediate. The triflate can then be reacted with an organotin reagent, for example, hexamethyldistannane in the presence of a palladium catalyst, for example, tetrakis(triphenylphosphine)palladium(0) and lithium chloride, to give the trimethylstannate intermediate (see, e.g., Scott and Stille, 1986). The organotin intermediate can then be fluorinated using a source of F⁺, for example, Selectfluor® (see, e.g., Matthews et al., 2003). The fluorocyclohexene can then be reacted further to give the mono-fluoroperhexiline as described in Scheme 1.

An example of such a method is shown in the following scheme.

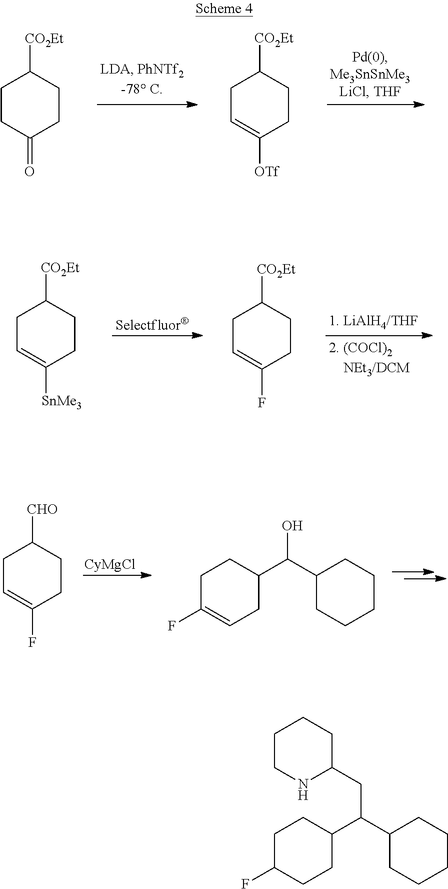

In another approach, the desired mono-fluoroperhexiline can be prepared by introduction of the fluorine group at a later stage, for example using the cyclohexyl(4-oxocyclohexyl)methyl benzoate shown in Scheme 1 and the fluorination methods described in Schemes 3 and 4, to give cyclohexyl(4-fluorocyclohex-3-enyl)methyl benzoate, which can then be further reacted by the methods shown in Scheme 1 to give the desired monofluoroperhexiline.

Examples of such methods are shown in the following schemes.

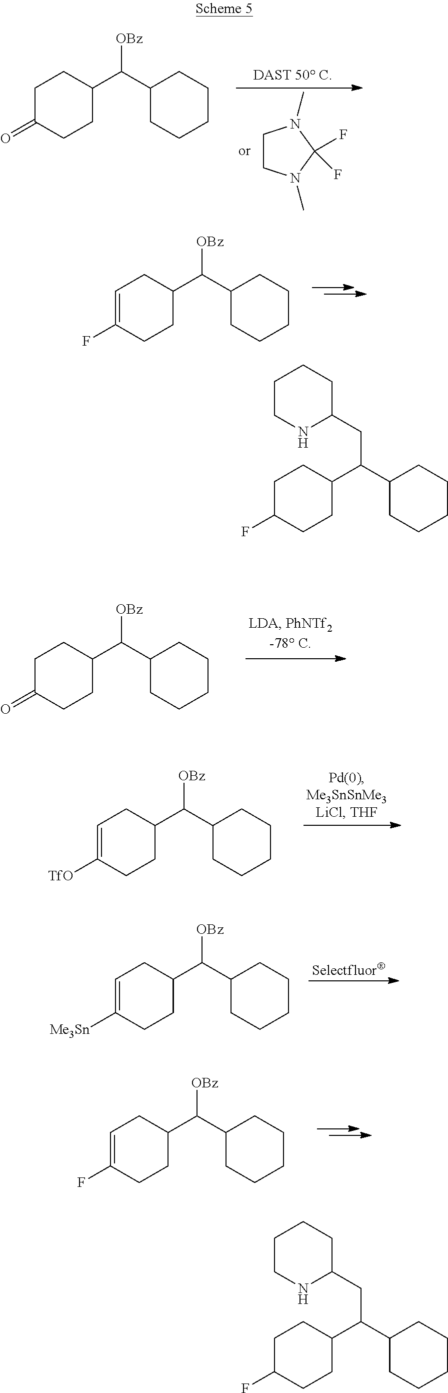

In another approach, the desired mono-fluoroperhexiline can be prepared by introduction of the fluorine group onto the cyclohexyl(4-oxocyclohexyl)methyl benzoate intermediate, prepared using a revised version of the method shown in Scheme 2. In this approach 2,4,6-triisopropyl-N-(1,4-dioxaspiro[4.5]decan-8-ylidene)benzenesulfonohydrazide is deprotonated with butyllithium to give 1,4-dioxaspiro[4.5]decane-8-carbaldehyde, which can then be reacted with cyclohexanemagnesium chloride as described in Scheme 1, to give the cyclohexane-cyclohexanone-methanol intermediate, which can then be further reacted as described in Scheme 5 to give the desired mono-fluoroperhexiline.

An example of such a method is shown in the following scheme.

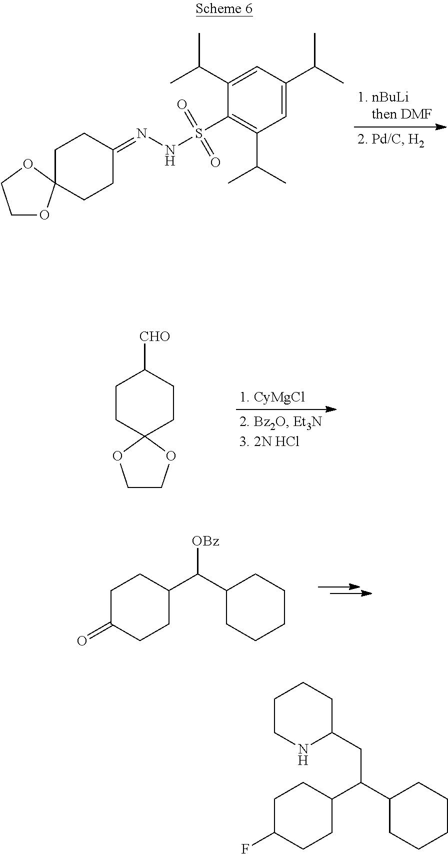

In another approach, the desired mono-fluoroperhexiline can be prepared by deprotonation of a benzenesulfonohydrazide-protected cyclohexanone and reaction with N-fluorobenzenesulfonimide (NFSI) to give the vinyl fluoride. The vinyl fluoride can then be further reacted using the methodology shown in Scheme 1, to give the desired mono-fluoroperhexiline.

For example, in one approach to preparation of the benzenesulfonohydrazide-protected cyclohexanone, 1,4-dioxaspiro[4.5]decane-8-carbaldehyde can first be prepared starting from 2,4,6-triisopropyl-N-(1,4-dioxaspiro[4.5]decan-8-ylidene)benzenesulfonohydrazide (Hu et al., 2006), which can be deprotonated and reacted with N,N-dimethylformamide to give 1,4-dioxaspiro[4.5]dec-7-ene-8-carbaldehyde, which is then hydrogenated to give the desired carbaldehyde, which may then be reacted with cyclohexanemagnesium chloride in dry tetrahydrofuran as described previously, to give the protected dicyclohexylmethanol skeleton. The protecting group can be removed under acidic conditions, the methanol alcohol protected with a suitable silyl protecting group, for example triethylsilyl and a hydrazide prepared from the ketone, for example by reaction with 2,4,6-triisopropylbenzenesulfonohydrazide. Deprotonation with a suitable base, for example n-butyllithium, and fluorination with NFSI gives an inseparable mixture of the fluorinated and protonated products. In order to separate these products, the mixture can be deprotected, for example the silane is removed with toluenesulfonic acid, and hydrogenated, for example with 10% palladium on carbon in an atmosphere of hydrogen. The cyclohexyl(4-fluorocyclohexyl)methanol can then be oxidised to the ketone, for example using Dess-Martin periodinane, and reacted with a lithiated 2-picoline solution as described previously, to give the mono-fluoro perhexiline as a mixture of diastereomers.

An example of such a method is shown in the following scheme.

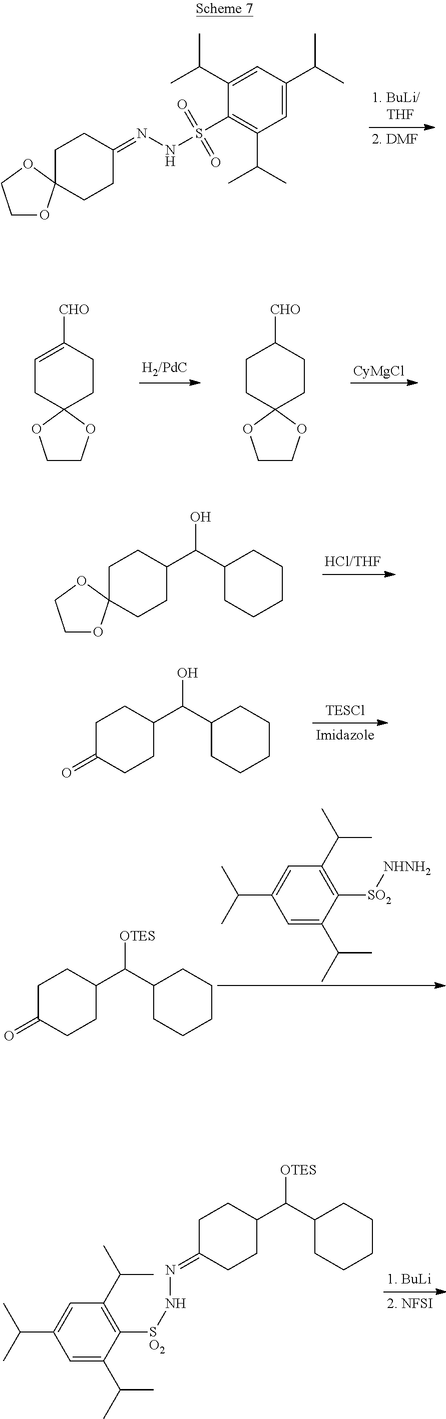

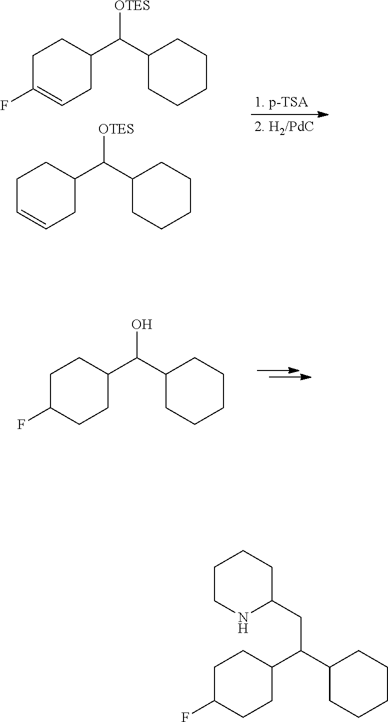

In another approach, bis-fluoroperhexiline can be prepared by modification of the above method to also include a fluorine atom on the second cyclohexyl ring. For example, the 1,4-dioxaspiro[4.5]decane-8-carbaldehyde intermediate, shown in Scheme 7, can be reacted with 2,4,6-triisopropyl-N-(1,4-dioxaspiro[4.5]decan-8-ylidene) benzenesulfonohydrazide, to give a dicyclohexylmethanol skeleton, with each ring bearing the 1,4-dioxaspiro protecting group which may then be removed, using the methodology shown above, to give the diketone. The diketone may then be converted to the bis-fluoro derivative by the same method as shown in Scheme 7.

An example of such a method is shown in the following scheme.

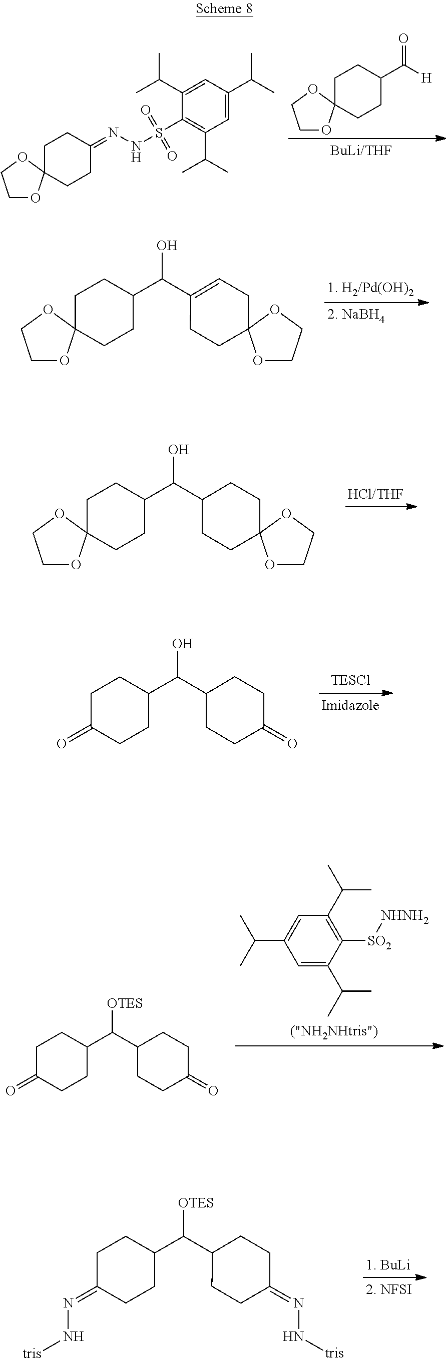

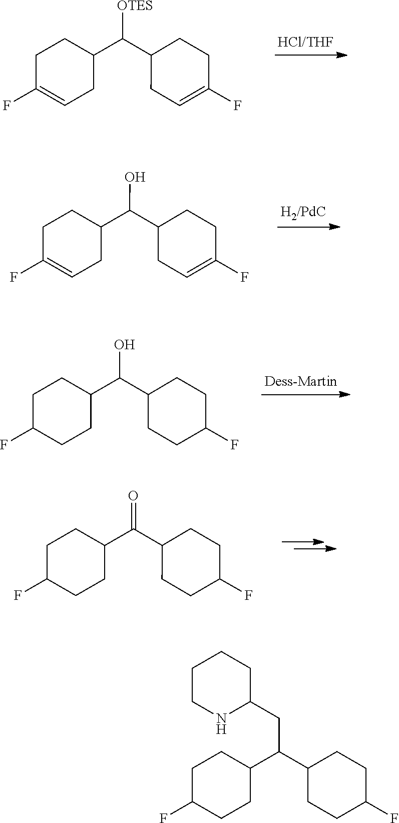

In another approach, a bis-fluoroperhexiline can be prepared by modification of the above methods to include a fluorine on the second cyclohexyl ring. For example, the 4-fluorocyclohex-3-enecarbaldehyde intermediate shown in Scheme 3 can be selectively hydrogenated, for example, in the presence of 10% Pd/C, and reacted with 2,4,6-triisopropyl-N-(1,4-dioxaspiro[4.5]decan-8-ylidene)benzenesulfonohydrazide to give the (4-fluorocyclohexyl)(1,4-dioxaspiro[4.5]dec-7-en-8-yl)methanol, analogous to the intermediate shown in Scheme 2. This intermediate can then be deprotected to provide the cyclohexanone moiety required for fluorination as described in Scheme 2. At this stage, either one or two fluorine groups can be introduced, either by further use of the fluorination method shown in Scheme 2, or by the methods shown in Scheme 5. Further reaction, using the methods shown in Schemes 1 and 2 can be used to give a final perhexiline product bearing either two or three fluorine groups.

Examples of such methods are shown in the following schemes.

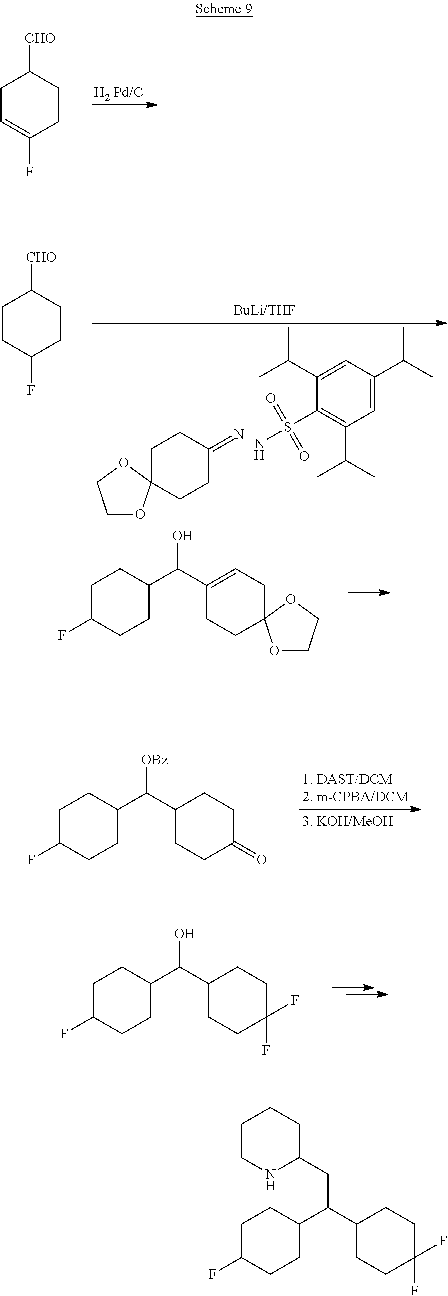

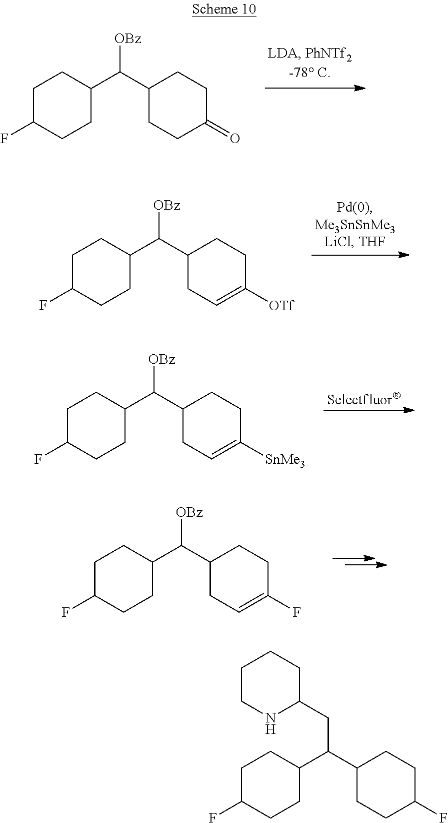

Enantiomerically-pure fluoroperhexilines, with a single chiral centre, can also be accessed via the intermediates shown in the above schemes. For example, in one approach, bis(4,4-difluorocyclohexyl)methanone (Scheme 2) can be reacted with 2-chloro-6-methylpyridine, to give an intermediate which can then be reacted with a chiral auxiliary, for example (R)-4-isopropyloxazolidinone in the presence of N,N-dimethylethylenediamine (DMEDA), CuI and K₂CO₃ (see, e.g., Glorius et al., 2004; Schelper et al., 2004). Hydrogenation of this intermediate will give exclusively the (S)-perhexiline product.

An example of such a method is shown in the following scheme.

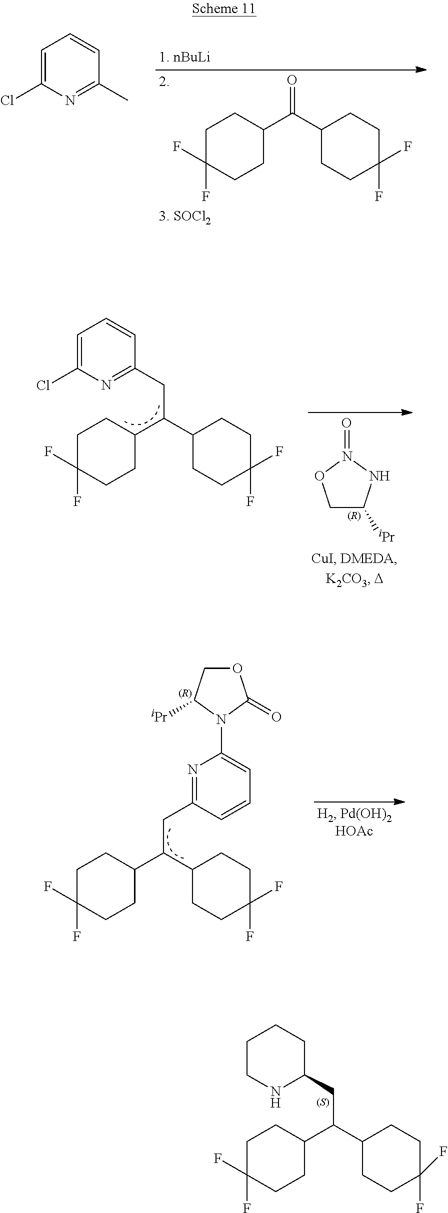

Enantiomerically-pure fluoroperhexilines, with two chiral centres, can also be accessed via the intermediates shown in the above schemes. For example, in one approach, cyclohexyl(4,4-difluorocyclohexyl)methanone (Scheme 1) can be reacted with 2-chloro-6-methylpyridine, to give an intermediate which can then be reacted with a chiral auxiliary, for example (S)-4-isopropyloxazolidinone in the presence of N,N-dimethylethylenediamine (DMEDA), CuI and K₂CO₃ (see, e.g., Schelper et al., 2004). Hydrogenation of this intermediate will give a diasteriomeric mixture of the (R,S)- and (R,R)-perhexiline products, which can be separated by column chromatography or HPLC (see, e.g., Heitbaum et al., 2010).

An example of such a method is shown in the following scheme.

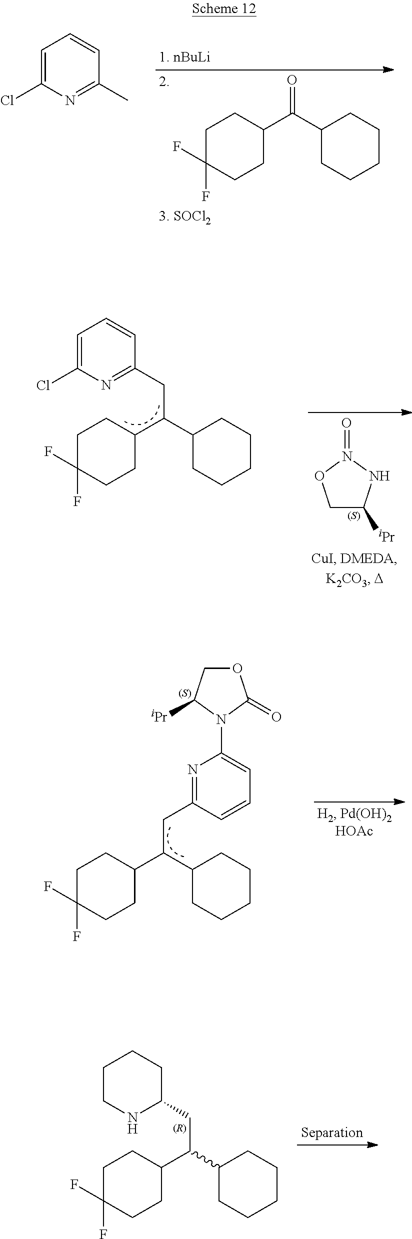

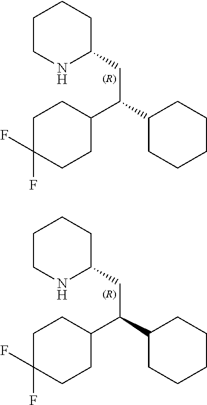

### Compositions

One aspect of the present invention pertains to a composition (e.g., a pharmaceutical composition) comprising an FPER compound, as described herein, and a pharmaceutically acceptable carrier, diluent, or excipient.

In one embodiment, the composition further comprises one or more (e.g., 1, 2, 3, 4) additional therapeutic agents, as described herein.

Another aspect of the present invention pertains to a method of preparing a composition (e.g., a pharmaceutical composition) comprising admixing an FPER compound, as described herein, and a pharmaceutically acceptable carrier, diluent, or excipient.

Another aspect of the present invention pertains to a method of preparing a composition (e.g., a pharmaceutical composition) comprising admixing an FPER compound, as described herein; one or more (e.g., 1, 2, 3, 4) additional therapeutic agents, as described herein; and a pharmaceutically acceptable carrier, diluent, or excipient.

### Uses

The FPER compounds, as described herein, are useful, for example, in the treatment of disorders (e.g., diseases) including, for example, those which are known to be treated with, or known to be treatable with, perhexiline, including, for example, the disorders (e.g., diseases) described herein.

### Use in Methods of Inhibiting Carnitine Palmitoyltransferase (CPT)

One aspect of the present invention pertains to a method of inhibiting carnitine palmitoyltransferase (CPT), in vitro or in vivo, comprising contacting the CPT with an effective amount of an FPER compound, as described herein.

One aspect of the present invention pertains to a method of inhibiting carnitine palmitoyltransferase (CPT) in a cell, in vitro or in vivo, comprising contacting the cell with an effective amount of an FPER compound, as described herein.

One of ordinary skill in the art is readily able to determine whether or not a candidate compound inhibits CPT. For example, suitable assays are described herein or are known in the art.

In one embodiment, the CPT is CPT-1.

In one embodiment, the CPT is CPT-1A.

In one embodiment, the CPT is CPT-1B.

In one embodiment, the CPT is CPT-1C.

In one embodiment, the CPT is CPT-2.

In one embodiment, the method is performed in vitro.

In one embodiment, the method is performed in vivo.

In one embodiment, the FPER compound is provided in the form of a pharmaceutically acceptable composition.

### Use in Methods of Therapy

Another aspect of the present invention pertains to an FPER compound, as described herein, for use in a method of treatment of the human or animal body by therapy, for example, for use a method of treatment of a disorder (e.g., a disease) as described herein.

Another aspect of the present invention pertains to an FPER compound, as described herein, in combination with one or more (e.g., 1, 2, 3, 4) additional therapeutic agents, as described herein, for use in a method of treatment of the human or animal body by therapy, for example, for use a method of treatment of a disorder (e.g., a disease) as described herein.

### Use in the Manufacture of Medicaments

Another aspect of the present invention pertains to use of an FPER compound, as described herein, in the manufacture of a medicament for treatment, for example, treatment of a disorder (e.g., a disease) as described herein.

In one embodiment, the medicament comprises the FPER compound.

Another aspect of the present invention pertains to use of an FPER compound, as described herein, and one or more (e.g., 1, 2, 3, 4) additional therapeutic agents, as described herein, in the manufacture of a medicament for treatment, for example, treatment of a disorder (e.g., a disease) as described herein.

In one embodiment, the medicament comprises the FPER compound and the one or more (e.g., 1, 2, 3, 4) additional therapeutic agents.

### Methods of Treatment

Another aspect of the present invention pertains to a method of treatment, for example, of a disorder (e.g., a disease) as described herein, comprising administering to a patient in need of treatment a therapeutically effective amount of an FPER compound, as described herein, preferably in the form of a pharmaceutical composition.

Another aspect of the present invention pertains to a method of treatment, for example, of a disorder (e.g., a disease) as described herein, comprising administering to a patient in need of treatment a therapeutically effective amount of an FPER compound, as described herein, preferably in the form of a pharmaceutical composition, and one or more (e.g., 1, 2, 3, 4) additional therapeutic agents, as described herein, preferably in the form of a pharmaceutical composition.

### Conditions Treated: Disorders Ameliorated by the Inhibition of CPT

In one embodiment (e.g., of use in methods of therapy, of use in the manufacture of medicaments, of methods of treatment), the treatment is treatment of a disorder (e.g., a disease) that is ameliorated by the inhibition of carnitine palmitoyltransferase (CPT).

In one embodiment, the CPT is CPT-1.

In one embodiment, the CPT is CPT-1A.

In one embodiment, the CPT is CPT-1B.

In one embodiment, the CPT is CPT-1C.

In one embodiment, the CPT is CPT-2.

### Conditions Treated: Disorders Ameliorated by the Inhibition of Fatty Acid Oxidation, Etc.

In one embodiment, the treatment is treatment of a disorder (e.g., a disease) that is ameliorated by inhibition of fatty acid oxidation (e.g., β-oxidation).

In one embodiment, the treatment is treatment of a disorder (e.g., a disease) that is characterised by impaired cardiac energetics.

In one embodiment, the treatment is treatment of a disorder (e.g., a disease) that is characterised by oxygen deficiency.

In one embodiment, the treatment is treatment of ischaemia.

### Conditions Treated: Cardiovascular Disorders

In one embodiment, the treatment is treatment of a cardiovascular disorder (e.g., a cardiovascular disease).

In one embodiment, the treatment is treatment of angina pectoris; heart failure (HF); left or right ventricular failure; pulmonary heart disease; ischaemic heart disease (IHD); cardiomyopathy; cardiac dysrhythmia; stenosis of a heart valve; hypertrophic cardiomyopathy (HCM); or coronary heart disease.

In one embodiment, the treatment is treatment of angina pectoris (also known as angina), for example, angina pectoris caused by coronary heart disease; angina pectoris caused by ischaemia; severe angina pectoris; or unresponsive or refractory angina pectoris.

In one embodiment, the treatment is treatment of heart failure (HF), for example, heart failure caused by ischaemia; congestive heart failure; chronic heart failure; moderate heart failure; systolic heart failure; diastolic heart failure; or diastolic heart failure with left ventricular injury.

In one embodiment, the treatment is treatment of left or right ventricular failure, for example, of various etiologies.

In one embodiment, the treatment is treatment of pulmonary heart disease, for example, pulmonary heart disease caused by pulmonary hypertension; pulmonary heart disease caused by chronic obstructive lung disease; or pulmonary heart disease caused by emphysema.

In one embodiment, the treatment is treatment of ischaemic heart disease (IHD), for example, ischaemic heart disease caused by coronary heart disease; ischaemic heart disease caused by obstruction of the coronary artery; ischaemic heart disease caused by spasm of the coronary artery; severe ischaemic heart disease (e.g., in a patient awaiting coronary revascularisation); or refractory ischaemic heart disease (e.g., in a patient with ischaemic symptoms refractory to other therapeutic measures).

In one embodiment, the treatment is treatment of cardiomyopathy, including, for example, cardiomyopathy due to ischaemic heart disease; or cardiomyopathy due to hypertension.

In one embodiment, the treatment is treatment of cardiac dysrhythmia (also known as cardiac arrhythmia or irregular heartbeat), for example, cardiac dysrhythmia caused by ischaemia.

In one embodiment, the treatment is treatment of stenosis of a heart valve, for example, aortic stenosis, for example, inoperable aortic stenosis.

In one embodiment, the treatment is treatment of hypertrophic cardiomyopathy (HCM), for example, symptomatic non-obstructive hypertrophic cardiomyopathy.

In one embodiment, the treatment is treatment of coronary heart disease.

### Conditions Treated: Metabolic-Related Disorders

In one embodiment, the treatment is treatment of a metabolic-related disorder, for example, diabetes (e.g., type 1 diabetes, type 2 diabetes, diabetes associated with heart failure); hyperglycemia; hyperlipidemia; hypertriglyceridemia; dyslipidemia; syndrome X (also known as metabolic syndrome); or obesity.

### Conditions Treated: Cancer

In one embodiment, the treatment is treatment of cancer.

In one embodiment, the treatment is treatment of: acute myeloid leukaemia; adrenal gland cancer; biliary tract cancer; bladder cancer; bone cancer; bowel cancer; brain cancer; breast cancer; colon cancer; colorectal cancer; endometrial cancer; gastrointestinal cancer; genito-urinary cancer; glioma; glioblastoma; gynaecological cancer; head cancer; Hodgkin's disease; Kaposi's sarcoma; kidney cancer; large bowel cancer; leukaemia; liver cancer; lung cancer; lymphoma; lymphocytic leukaemia (lymphoblastic leukaemia); malignant melanoma; mediastinum cancer; melanoma; myeloma; myelogenous leukaemia (myeloid leukaemia); nasopharyngeal cancer; neck cancer; nervous system cancer; non-Hodgkin's lymphoma; non-small cell lung cancer; oesophagus cancer; osteosarcoma; ovarian cancer; pancreatic cancer; prostate cancer; rectal cancer; renal cell carcinoma; sarcoma; skin cancer; small bowel cancer; small cell lung cancer; soft tissue sarcoma; squamous cancer; stomach cancer; testicular cancer; or thyroid cancer.

In one embodiment, the treatment is treatment of glioblastoma or acute myeloid leukaemia.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment of angina pectoris includes the prophylaxis of angina pectoris, reducing the incidence of angina pectoris, reducing the severity of angina pectoris, alleviating the symptoms of angina pectoris, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, anti-anginal agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

One aspect of the present invention pertains to a compound as described herein, in combination with one or more additional therapeutic agents, as described below.

The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

### Additional Therapeutic Agents for Use in Combination Therapy

Known drugs which are used in combination with perhexiline may also be used in combination therapy with a FPER compound as described herein. Examples of such drugs include: amiodarone, amitriptyline, atenolol, captopril, diltiazem, digoxin, enalaprilat, fluoxetine, glibenclamide, metformin, prednisolone, prednisone, sertraline, theophylline, verapamil, and warfarin. (See, e.g., Sallustio et al., 2002.)

Diuretics may be used in the treatment of heart failure, and as such may be used in combination with an FPER compound to give an additive therapeutic benefit. Examples of such diuretics include: thiazides (e.g., bendroflumethiazide, chlorthalidone, metolazone), loop diuretics (e.g., bumetanide, furosemide, torasemide); potassium-sparing diuretics (e.g., amiloride, triamterene); aldosterone antagonists (e.g., canrenone, eplerenone, mexrenone, prorenone and spironolactone).

Antihypertensives may be used in the treatment of high blood pressure, which is associated with a number of cardiovascular diseases, including heart failure, angina, and risk of heart attack, and other aspects of cardiac disease, including diabetes and cardiac arrhythmia, and as such may be used in combination with an FPER compound to give an additive therapeutic benefit. Examples of such antihypertensives include: angiotensin converting enzyme inhibitors (ACE Inhibitors) (e.g., captopril, cilazapril, enalapril, imidapril, lisinopril, moexipril, perindopril, quinapril, ramipril and trandolapril); calcium channel blockers (e.g., amlodipine, aranidipine, azelnidipine, barnidipine, benidipine, bepridil, cilnidipine, cinalong, clevidipine, diltiazem, efonidipine, felodipine, fendiline, isradipine, lacidipine, lercanidipine, manidipine, mibefradil, nicardipine, nifedipine, nilvadipine, nimodipine, nisoldipine, nitrendipine, pranidipine, verapamil, and ziconotide); beta blockers (e.g., acebutolol, alprenolol, atenolol, betaxolol, bisoprolol, bucindolol, carteolol, carvedilol, celiprolol, esmolol, labetalol, metoprolol, nadolol, nebivolol, oxprenolol, penbutolol, pindolol, propranolol, sotalol, and timolol); angiotensin II receptor antagonists (e.g., azilsartan, candesartan, eprosartan, irbesartan, losartan, olmesartan, telmisartan, and valsartan).

Hypolipidaemics may be used in the treatment of cardiovascular diseases and diabetes, and as such may be used in combination with an FPER compound to give an additive therapeutic benefit. Examples of such hypolipidaemics include: statins (e.g., atorvastatin, fluvastatin, lovastatin, pitavastatin, pravastatin, rosuvastatin, and simvastatin); fibrates (e.g., bezafibrate, ciprofibrate, clofibrate, gemfibrozil, and fenofibrate); niacin and derivatives including acipimox; selective inhibitors of dietary cholesterol absorption (e.g., ezetimibe); microsomal triglyceride transfer protein (MTP) inhibitors (e.g., lomitapide); and other agents which reduce the absorption of dietary fats (e.g., orlistat).

Agents which control glucose levels and insulin production and sensitivity may be used in the treatment of type II diabetes, and as such may be used in combination with an FPER compound to give an additive therapeutic benefit. Examples of such diabetes therapies include: acarbose, acetohexamide, chlorpropamide, exenatide, gliclazide, glimepiride, glipizide, glyburide, linagliptin, liraglutide, metformin, miglitol, pioglitazone, repaglinide, nateglinide, saxagliptin, sitagliptin, taspoglutide, tolazamide, tolbutamide, vildagliptin, and voglibose.

Anticoagulants may be used in the treatment of a number of cardiovascular conditions, including myocardial infarction, hypertension, diabetes, and heart failure, and as such may be used in combination with an FPER compound to give an additive therapeutic benefit. Examples of such anticoagulants include: acenocoumarol, dabigatran, phenindione, rivaroxaban, and warfarin.

Cardiac dysrhythmias are commonly associated with cardiovascular diseases and thus anti-arrhythmic agents may be used in the treatment of cardiovascular diseases, and as such may be used in combination with an FPER compound to give an additive therapeutic benefit. Examples of such anti-arrhythmic agents include: potassium channel blockers (e.g., amiodarone, dofetilide, dronedarone, ibutilide, lidocaine, and sotalol); sodium channel blockers (e.g., disopyramide, encainide, flecainide, mexiletine, moricizine, phenytoin, procainamide, propafenone, quinidine, and tocainide); calcium channel blockers (e.g., amlodipine, aranidipine, azelnidipine, barnidipine, benidipine, bepridil, cilnidipine, cinalong, clevidipine, diltiazem, efonidipine, felodipine, fendiline, fluspirilene, isradipine, lacidipine, lercanidipine, manidipine, mibefradil, nicardipine, nifedipine, nilvadipine, nimodipine, nisoldipine, nitrendipine, pranidipine, verapamil, and ziconotide).

Organic nitrates and nitrites may be used in the treatment of angina and as such may be used in combination with an FPER compound to give an additive therapeutic benefit. Examples of such organic nitrates include: isosorbide dinitrate, isosorbide mononitrate, nitroglycerin, and amyl nitrite.

Digitalis may be used in the treatment of heart failure, and as such may be used in combination with an FPER compound to give an additive therapeutic benefit.

### Other Uses

The FPER compounds described herein may also be used as part of an in vitro assay, for example, in order to determine whether a candidate host is likely to benefit from treatment with the compound in question.

The FPER compounds described herein may also be used as a standard, for example, in an assay, in order to identify other compounds, other anti-anginal agents, etc.

### Kits

One aspect of the invention pertains to a kit comprising (a) an FPER compound as described herein, or a composition comprising an FPER compound as described herein, e.g., preferably provided in a suitable container and/or with suitable packaging; and (b) instructions for use, e.g., written instructions on how to administer the compound or composition.

In one embodiment, the kit further comprises one or more (e.g., 1, 2, 3, 4) additional therapeutic agents, as described herein.

The written instructions may also include a list of indications for which the active ingredient is a suitable treatment.

### Routes of Administration

The FPER compound or pharmaceutical composition comprising the FPER compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray, drops or from an atomiser or dry powder delivery device); ocular (e.g., by eyedrops); pulmonary (e.g., by inhalation or insufflation therapy using, e.g., an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In one preferred embodiment, the route of administration is oral (e.g., by ingestion). In one preferred embodiment, the route of administration is parenteral (e.g., by injection).

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the FPER compound to be administered alone, it is preferable to present it as a pharmaceutical formulation (e.g., composition, preparation, medicament) comprising at least one FPER compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one FPER compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/mL to about 10 μg/mL, for example, from about 10 ng/mL to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the FPER compounds, and compositions comprising the FPER compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular FPER compound, the route of administration, the time of administration, the rate of excretion of the FPER compound, the duration of the treatment, other drugs, compounds, and/or materials used in combination, the severity of the condition, and the species, sex, age, weight, condition, general health, and prior medical history of the patient. The amount of FPER compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the FPER compound is in the range of about 50 μg to about 20 mg (more typically about 100 μg to about 10 mg) per kilogram body weight of the subject per day. Where the compound is a salt, an ester, an amide, a prodrug, or the like, the amount administered is calculated on the basis of the parent compound and so the actual weight to be used is increased proportionately.

It may be noted that, currently, perhexiline is administered as racemic perhexiline maleate, at an initial dosage of 100 mg/day, and increased or decreased if required. For “poor metabolizers” (PM), the dosage is typically 10-25 mg/day. For “extensive metabolizers” (EM), the dosage is typically 100-250 mg/day. For “ultra metabolizers” (UM), the dosage is typically 300-500 mg/day. The optimal steady state plasma concentration of perhexiline is about 0.15-0.6 mg/L.

### Examples

### Chemical Synthesis

The following examples are provided solely to illustrate the present invention and are not intended to limit the scope of the invention, as described herein.

### Synthesis 1

### 4-(tert-Butyldimethylsilyloxy)cyclohex-1-enecarbaldehyde (1)

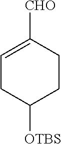 [SMILES: CC(C)(C)[Si](C)(C)OC1CC=C(C=O)CC1]

To a solution of N-(4-(tert-butyldimethylsilyloxy)cyclohexylidene)-4-methylbenzenesulfonohydrazide (prepared according to Fan et al., 2004) (550 mg, 1.388 mmol) in hexane-tetramethylethylenediamine (20 mL, 1:1, v/v) was added a solution of n-BuLi (3.47 mL, 5.55 mmol, 1.6 M in hexanes) at −78° C. The resulting red solution was stirred for 10 min prior to warming to 23° C. for 4 h, after which the solution was cooled to −20° C. and N,N-dimethylformamide (0.58 mL, 7.52 mmol) was added. The reaction mixture was stirred at 0° C. for 1 h before it was quenched with a saturated solution of NH₄Cl (10 mL). Following extraction with Et₂O (3×20 mL), the combined organic extracts were dried over Na₂SO₄ and evaporated in vacuo to give a yellow residue, which was purified by flash chromatography (silica gel, hexanes:EtOAc 4:1) to furnish aldehyde 1 (294 mg, 88%) as a pale yellow oil.

¹H NMR (400 MHz, CDCl₃) δ=9.41 (s, 1H), 6.66 (tt, J=3.3, 1.5 Hz, 1H), 4.05-3.92 (m, 1H), 2.63-2.47 (m, 1H), 2.47-2.34 (m, 1H), 2.34-2.22 (m, 1H), 2.22-2.09 (m, 1H), 1.81-1.69 (m, 1H), 1.69-1.55 (m, 1H), 0.86 (s, 9H), 0.059 (s, 3H), 0.059 (s, 3H); ¹³C NMR (101 MHz, CDCl₃) δ=193.6, 148.4, 141.0, 66.6, 36.0, 30.0, 25.8, 19.1, 18.1, −4.7, −4.8.

### Synthesis 2

### (4-(tert-Butyldimethylsilyloxy)cyclohex-1-enyl)(cyclohexyl)methanol (2)

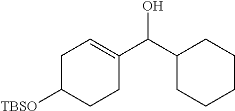 [SMILES: CC(C)(C)[Si](C)(C)OC1CC=C(C(O)C2CCCCC2)CC1]

To a solution of aldehyde 1 (270 mg, 1.123 mmol) in THF (10 mL) was added cyclohexylmagnesium chloride (1.35 mL, 1.35 mmol, 1.0 M in MeTHF) at 0° C. The resulting solution was stirred at 0° C. for 1 h before it was quenched with a saturated solution of NH₄Cl (10 mL). Following extraction with Et₂O (3×20 mL), the combined organic extracts were dried over Na₂SO₄ and evaporated in vacuo to give a yellow residue which was purified by flash column chromatography (silica gel; hexane:EtOAc, 4:1) to give alcohol 2 (350 mg, 96%; ca. 1.13:1 mixture of diastereoisomers determined by ¹H NMR) as a pale yellow oil.

¹H NMR (400 MHz, CDCl₃) δ=5.56-5.39 (m, 1H), 3.99-3.90 (m, 0.53; H), 3.85 (tdd, J=7.8, 5.2, 3.1 Hz, 0.47; H), 3.66-3.63 (m, 0.47; H), 3.63-3.61 (m, 0.53; H), 2.31-2.18 (m, 1H), 2.14-1.86 (m, 3H), 1.85-1.54 (m, 6H), 1.53-1.34 (m, 4H), 1.28-1.07 (m, 4H), 0.89 (s, 4.23; H), 0.86 (s, 4.77; H), 0.062 (s, 1.41; H), 0.056 (s, 1.41; H), 0.052 (s, 1.59; H), 0.04 (s, 1.59; H); ¹³C NMR (101 MHz, CDCl₃) δ=138.7, 138.5, 121.8, 121.7, 81.73, 81.0, 68.0, 67.1, 40.7, 40.7, 35.0, 34.6, 31.7, 30.9, 29.6, 29.5, 29.2, 28.9, 26.5, 26.2, 26.1, 26.0, 26.0, 25.9, 25.8, 22.3, 21.0, 18.14, 18.06, −4.6, −4.7.

### Synthesis 3

### Cyclohexyl(4-hydroxycyclohexyl)methyl benzoate (3)

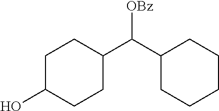

To a solution of alcohol 2 (326 mg, 1 mmol), 4-(dimethylamino)pyridine (12.1 mg, 0.1 mmol) and pyridine (250 μL, 3.09 mmol) in CH₂Cl₂ (2 mL) was added benzoyl chloride (145 μL, 1.25 mmol) at 0° C. The resulting reaction mixture was warmed to 23° C. and stirred for 24 h before it was quenched with a saturated solution of NaHCO₃ (10 mL) and diluted with Et₂O (30 mL). The layers were separated and the organic layer was washed with a solution of HCl (1 M, 2×5 mL) followed by brine (2×5 mL). The combined organic extracts were dried over Na₂SO₄ and evaporated in vacuo to give the crude benzoate compound, which was used directly without further purification.

Palladium on activated charcoal (30 mg, 10% wt/wt) was suspended in a solution of the crude benzoate compound in MeOH (10 mL) at 23° C. under an atmosphere of hydrogen (1 atm) for 16 h, before it was filtered through a pad of Celite® and the filtrate was concentrated in vacuo. The residue was dissolved in MeOH—CH₂Cl₂ (10 mL, 1:1, v/v) at 23° C. and a solution of HCl (1 M, 1 mL) was added. The resulting mixture was stirred for 3 h before it was quenched with a saturated solution of NaHCO₃ (15 mL) and diluted with CH₂Cl₂ (10 mL). The layers were separated, and the aqueous layer was extracted with CH₂Cl₂ (4×5 mL). The combined organic layers were dried over Na₂SO₄ and concentrated in vacuo to afford the corresponding alcohol. The crude alcohol 3 was passed through a short silica plug (EtOAc) and concentrated in vacuo prior to use.

### Synthesis 4

### Cyclohexyl(4-oxocyclohexyl)methyl benzoate (4)

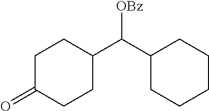

To a solution of the crude alcohol 3 in CH₂Cl₂ (5 mL) was added Dess-Martin periodinane (636 mg, 1.5 mmol) at 23° C. The reaction mixture was stirred for 16 h before it was quenched with a saturated solution of Na₂SO₃ (5 mL). The resulting mixture was extracted with CH₂Cl₂ (3×10 mL), the combined organic layers were dried over Na₂SO₄ and concentrated in vacuo. The residue was purified by flash column chromatography (silica gel, hexanes:EtOAc 3:1→1:1) to give ketone 4 (215 mg, 68% for 4 steps).

¹H NMR (400 MHz, CDCl₃) δ=8.08-8.00 (m, 2H), 7.63-7.51 (m, 1H), 7.50-7.40 (m, 2H), 5.04 (t, J=5.9 Hz, 1H), 2.47-2.25 (m, 4H), 2.25-2.14 (m, 1H), 2.14-1.98 (m, 2H), 1.81-1.50 (m, 7H), 1.36-1.04 (m, 6H); ¹³C NMR (101 MHz, CDCl₃) δ=211.2, 166.4, 133.0, 130.1, 129.6, 128.4, 80.0, 40.5, 40.4, 39.2, 37.0, 29.8, 29.5, 27.8, 27.0, 26.2, 26.1, 25.9.

### Synthesis 5

### Cyclohexyl(4,4-difluorocyclohexyl)methanol (5)

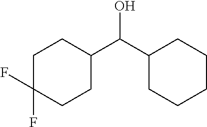 [SMILES: OC(C1CCCCC1)C1CCC(F)(F)CC1]

To a solution of ketone 4 (340 mg, 1.08 mmol) in CH₂Cl₂ (5 mL) was added diethylaminosulfur trifluoride (286 μL, 2.16 mmol) at 0° C. The resulting mixture was stirred for 24 h at 23° C. before it was quenched with a saturated solution of NaHCO₃ (10 mL). The resulting mixture was extracted with CH₂Cl₂ (3×10 mL), the combined organic layers were dried over Na₂SO₄ and concentrated in vacuo to afford the corresponding gem-difluoro- and vinyl fluoride as an inseparable mixture. The residue was passed through a short silica plug (EtOAc) and concentrated in vacuo prior to use.

To a solution of the mixture of the gem-difluoro- and vinyl fluoride in CH₂Cl₂ at 23° C. was added 3-chloroperbenzoic acid (242 mg, 1.08 mmol, 77% in H₂O). The resulting reaction mixture was stirred for 16 h at 23° C. before it was quenched with a saturated solution of Na₂SO₃ (5 mL). The resulting mixture was extracted with CH₂Cl₂ (3×10 mL), the combined organic layers were dried over Na₂SO₄ and concentrated in vacuo to give a crude mixture of the gem-difluoride and fluoroepoxide. These were re-suspended in THF-MeOH (10 mL, 4:1, v/v) and a solution of KOH (2 mL, 10% aq.) was added at 23° C. The mixture was warmed to 60° C. for 6 h before being diluted with H₂O (20 mL). The resulting mixture was extracted with CH₂Cl₂ (3×10 mL), the combined organic layers were dried over Na₂SO₄ and concentrated in vacuo. The residue was purified by flash column chromatography (silica gel, hexanes:EtOAc 5:1→1:1) to afford alcohol 5 (138 mg, 55% for 3 steps) as a colourless oil.

¹H NMR (400 MHz, CDCl₃) δ=3.13 (dd, J=11.8, 5.3 Hz, 1H), 2.18-2.04 (m, 2H), 1.97-1.86 (m, 1H), 1.84-1.59 (m, 7H), 1.59-1.32 (m, 6H), 1.29-0.97 (m, 5H); ¹⁹F NMR (376 MHz, CDCl₃) δ=−91.26 (d, J=234.4 Hz, 1F), −102.55 (dtt, J=234.3, 33.8, 10.1 Hz, 1F).

### Synthesis 6

### Cyclohexyl(4,4-difluorocyclohexyl)methanone (6)

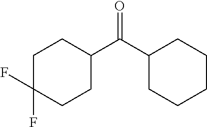 [SMILES: O=C(C1CCCCC1)C1CCC(F)(F)CC1]

To a solution of alcohol 5 (280 mg, 1.205 mmol) in CH₂Cl₂ (10 mL) was added Dess-Martin periodinane (767 mg, 1.808 mmol) at 23° C. The reaction mixture was stirred for 16 h before it was quenched with a saturated solution of Na₂SO₃ (10 mL). The resulting mixture was extracted with CH₂Cl₂ (4×10 mL), combined organic layers were dried over Na₂SO₄ and concentrated in vacuo. The residue was purified by flash column chromatography (silica gel, hexanes:EtOAc 5:1→2:1) to give ketone 6 (227 mg, 82%).

¹H NMR (400 MHz, CDCl₃) δ=2.64-2.47 (m, 2H), 2.21-2.08 (m, 2H), 1.92-1.66 (m, 10H), 1.43-1.20 (m, 6H); ¹³C NMR (101 MHz, CDCl₃) δ=215.1 (d, J=1.6 Hz), 122.7 (t, J=241.1 Hz), 49.2, 47.3, 33.0 (d, J=24.0 Hz), 32.7 (d, J=24.0 Hz), 28.6, 25.8, 25.6, 24.9 (d, J=1.5 Hz), 24.8 (d, J=1.5 Hz); ¹⁹F NMR (376 MHz, CDCl₃) δ=−93.02 (d, J=240.6 Hz, 1F), −100.63 (d, J=240.6 Hz, 1F).

### Synthesis 7

### (E) and (2)-2-(2-Cyclohexyl-2-(4,4-difluorocyclohexyl)vinyl)pyridine (7) and 2-(2-Cyclohexyl-2-(4,4-difluorocyclohexylidene)ethyl)pyridine/2-(2-Cyclohexylidene-2-(4,4-difluorocyclohexyl)ethyl)pyridine (8)

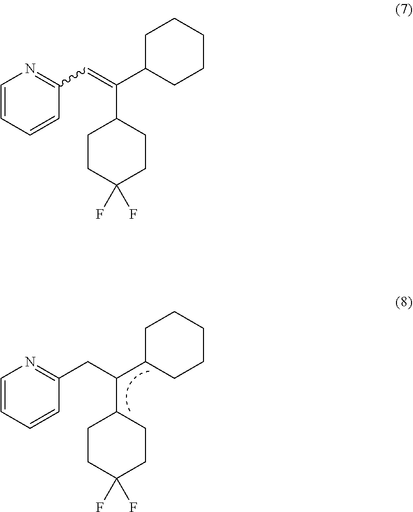 [SMILES: FC1(F)CCC(C(=Cc2ccccn2)C2CCCCC2)CC1 ; FC1(F)CCC(C(Cc2ccccn2)C2CCCCC2)CC1]

To a solution of 2-picoline (243 μL, 2.453 mmol) in THF (5 mL) was added a solution of n-BuLi (1.72 mL, 2.58 mmol, 1.5 M in hexanes) at −78° C. The resulting brick-red solution was warmed to −20° C. and stirred for 30 min before it was cooled to −78° C. affording a lithiated 2-picoline solution. A THF (5 mL) solution of ketone 6 (452 mg, 1.963 mmol) was cannulated to the lithiated 2-picoline solution at −78° C. and stirred for 30 min prior to adding SOCl₂ (190 μL, 2.459 mmol). The reaction mixture was warmed to 23° C. and stirred for 1 h before it was quenched with a saturated solution of NaHCO₃ (10 mL). The resulting mixture was extracted with CH₂Cl₂ (3×10 mL), combined organic layers were dried over Na₂SO₄ and concentrated in vacuo. The residue was purified by flash column chromatography (silica gel, hexanes:EtOAc 10:1→2:1) to obtain compound 7 as a 1:1.13 mixture of two stereoisomers (419 mg, 70%), and compound 8 as a 1:1.35 mixture of regioisomers (144 mg, 24%).

Compound 7: ¹H NMR (400 MHz, CDCl₃) δ=8.60-8.52 (m, 1H), 7.60 (tt, J=7.6, 2.5 Hz, 1H), 7.17-7.05 (m, 2H), 6.34 (s, 0.47; H), 6.33 (s, 0.53; H), 3.51-3.42 (m, 0.53; H), 3.16 (tt, J=11.7, 3.3 Hz, 0.47; H), 2.25-1.94 (m, 3H), 1.89-1.52 (m, 11H), 1.45-1.08 (m, 5H); ¹³C NMR (101 MHz, CDCl₃) δ=157.5, 157.3, 156.13, 156.11, 155.19, 155.17, 135.83, 135.77, 123.9, 123.3 (t, J=241.0 Hz), 123.1 (t, J=241.0 Hz), 123.7, 123.5, 120.7, 120.6, 40.5, 39.6, 38.40, 38.38, 37.85, 37.84, 35.0, 34.2 (dd, J=25.7, 22.3 Hz), 33.7 (dd, J=25.5, 22.2 Hz), 30.9, 30.8, 30.6, 26.9, 26.7, 26.6, 26.12, 26.09, 26.0; ¹⁹F NMR (376 MHz, CDCl₃) δ=−90.2 (d, J=234.7 Hz, 0.53F), −91.2 (d, J=236.0 Hz, 0.47F), −101.8-−102.3 (m, 0.47F), −102.4-−103.0 (m, 0.53F).

Compound 8: ¹H NMR (400 MHz, CDCl₃) δ=8.53-8.45 (m, 1H), 7.53 (tt, J=7.7, 1.7 Hz, 1H), 7.11-7.00 (m, 2H), 3.64 (s, 0.85; H), 3.60 (s, 1.15; H), 2.73-2.55 (m, 1H), 2.48 (t, J=6.7 Hz, 1H), 2.33-2.27 (m, 1H), 2.25 (t, J=6.5 Hz, 1H), 2.09 (d, J=6.0 Hz, 1H), 2.06-1.78 (m, 3H), 1.78-1.31 (m, 9H), 1.30-1.14 (m, 2H); ¹³C NMR (101 MHz, CDCl₃) δ=161.5, 161.1, 149.15, 149.12, 137.7, 136.1, 136.0, 134.4, 131.1, 128.3, 128.2, 123.5 (t, J=240.8 Hz), 123.0 (t, J=240.8 Hz), 121.7, 120.8, 120.7, 41.4, 38.91, 38.90, 37.0, 36.7, 35.1 (t, J=23.5 Hz), 34.6 (t, J=23.5 Hz), 33.9 (dd, J=25.6, 22.1 Hz), 31.7, 31.5, 30.3, 28.6, 28.0, 27.6, 27.5, 26.9, 26.5, 25.9, 21.2 (t, J=5.6 Hz); ¹⁹F NMR (376 MHz, CDCl₃) δ=−90.5 (d, J=233.8 Hz, 0.57F), −97.1 (quint. J=14.0 Hz, 0.86F), −102.6 (dtt, J=234.8, 34.8, 10.0 Hz, 0.57F).

### Synthesis 8

### 2-(2-Cyclohexyl-2-(4,4-difluorocyclohexyl)ethyl)piperidine (9)

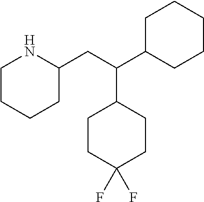 [SMILES: FC1(F)CCC(C(CC2CCCCN2)C2CCCCC2)CC1]

Palladium on activated charcoal (110 mg, 20% wt/wt) was suspended in a solution of a mixture of compounds 7 and 8 (560 mg, 1.834 mmol) in AcOH (10 mL) at 50° C. under an atmosphere of hydrogen (1 atm) for 60 h before filtering through a pad of Celite®. The filtrate was diluted with H₂O (20 mL) and the pH value of the aqueous solution was adjusted to 10 by NaHCO₃ prior to the solution being extracted with CH₂Cl₂ (5×10 mL). The combined organic layers were dried over Na₂SO₄ and concentrated in vacuo. The residue was purified by flash column chromatography (silica gel, NH₄OH_{aq}:MeOH:CH₂Cl₂=2:10:88, v/v) to obtain compound 9 (540 mg, 94%; mixture of diastereoisomers) as a pale yellow oil.

¹H NMR (400 MHz, CDCl₃) δ=3.04 (dd, J=7.0, 4.9 Hz, 1H), 2.59 (tt, J=11.8, 5.9 Hz, 1H), 2.43-2.30 (m, 1H), 2.14-1.92 (m, 2H), 1.82-1.49 (m, 12H), 1.49-0.90 (m, 16H); ¹³C NMR (101 MHz, CDCl₃) δ=123.63 (t, J=240.6 Hz), 123.59 (t, J=240.6 Hz), 56.8, 56.7, 47.4, 43.95, 43.93, 40.80, 40.78, 40.2, 40.0, 38.3, 38.1, 36.7, 34.29 (d, J=3.9 Hz), 34.15 (d, J=3.9 Hz), 34.1 (d, J=3.1 Hz), 34.0 (d, J=3.1 Hz), 33.95 (d, J=2.7 Hz), 33.89 (d, J=2.7 Hz), 33.8 (d, J=3.9 Hz), 33.7 (d, J=2.7 Hz), 33.4, 33.3, 31.7, 29.8, 29.6, 27.6 (d, J=3.2 Hz), 27.5 (d, J=3.2 Hz), 27.0, 26.9, 26.8, 26.7, 26.5, 25.8 (d, J=9.6 Hz), 25.5 (d, J=9.6 Hz), 25.0; ¹⁹F NMR (376 MHz, CDCl₃) δ=−90.9 (d, J=234.0 Hz, 1F), −102.6 (dtt, J=234.0, 35.3, 10.0 Hz, 1F). m/z (ESI) 314.2 [M+H]⁺.

### Synthesis 9

### Cyclohexyl(4-(methoxymethoxy)cyclohexyl)methanol (10)

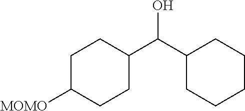

To a solution of cyclohexyl(4-(methoxymethoxy)cyclohexyl)methanone (1.02 g, 4.0 mmol) in MeOH (10 mL) was added NaBH₄ (230 mg, 6.07 mmol) at 0° C. The reaction mixture was stirred for 3 h before it was quenched with a saturated solution of NaHCO₃ (10 mL). The resulting mixture was extracted with CH₂Cl₂ (4×10 mL), the combined organic layers were dried over Na₂SO₄ and concentrated in vacuo. The residue was passed through a short silica plug (EtOAc) giving crude alcohol 10.

### Synthesis 10

### Cyclohexyl(4-(methoxymethoxy)cyclohexyl)methyl benzoate (11)

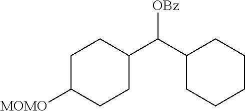

To a solution of alcohol and pyridine (500 μL, 6.18 mmol) in CH₂Cl₂ (5 mL) was added benzoyl chloride (560 μL, 4.82 mmol) at 0° C. The resulting reaction mixture was warmed to 23° C. and stirred for 16 h before it was quenched with a saturated solution of NaHCO₃ (10 mL) and diluted with Et₂O (40 mL). The layers were separated and the organic layer was washed with a solution of HCl (1 M, 2×5 mL) followed by brine (2×5 mL). The combined organic extracts were dried over Na₂SO₄ and evaporated in vacuo to afford the crude benzoate compound 11, which was used directly without further purification.

### Synthesis 11

### Cyclohexyl(4-hydroxycyclohexyl)methyl benzoate (12)

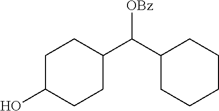

To a solution of crude benzoate compound in THF (10 mL) was added HCl (6 M aq., 2 mL) at 23° C. The resulting mixture was heated to 50° C. for 6 h before it was diluted with H₂O (5 mL) and quenched with a saturated solution of NaHCO₃ (10 mL). The resulting mixture was extracted with CH₂Cl₂ (4×10 mL), the combined organic layers were dried over Na₂SO₄ and concentrated in vacuo affording the crude alcohol 12.

### Synthesis 12

### Cyclohexyl(4-oxocyclohexyl)methyl benzoate (4)

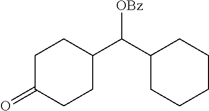

To a solution of the crude alcohol 12 in CH₂Cl₂ (10 mL) was added Dess-Martin periodinane (2.078 g, 4.9 mmol) at 23° C. The reaction mixture was stirred for 16 h before it was quenched with a saturated solution of Na₂SO₃ (10 mL). The resulting mixture was extracted with CH₂Cl₂ (4×10 mL), the combined organic layers were dried over Na₂SO₄ and concentrated in vacuo. The residue was purified by flash column chromatography (silica gel, hexanes:EtOAc 3:1→1:1) to afford ketone 4 (0.942 g, 75% for 4 steps). Analytical data as described in synthesis 5.

### Synthesis 13

### Ethyl 4-oxocyclohexanecarboxylate (13)

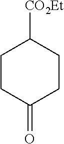 [SMILES: CCOC(=O)C1CCC(=O)CC1]

Powdered 3 Å molecular sieves (5 g) and pyridinium chlorochromate (2.23 g, 10.36 mmol) were suspended in a solution of ethyl 4-hydroxycyclohexanecarboxylate (1.19 g, 6.91 mmol; mixture of cis and trans isomers) in CH₂Cl₂ (20 mL) at 0° C. The resulting reaction mixture was stirred at 23° C. for 16 h before it was filtered through a pad of Celite®. The filtration cake was washed with CH₂Cl₂ (4×10 mL) and the filtrate was concentrated in vacuo. The residue was purified by flash column chromatography (silica gel, hexanes:EtOAc 5:1→1:1) to afford compound 13 (1.034 g, 88%) as a colourless oil.

¹H NMR (400 MHz, CDCl₃) δ=4.13 (q, J=7.1 Hz, 2H), 2.70 (tt, J=9.7, 4.0 Hz, 1H), 2.43 (dt, J=14.7, 5.4 Hz, 2H), 2.36-2.25 (m, 2H), 2.23-2.11 (m, 2H), 2.05-1.91 (m, 2H), 1.23 (t, J=7.1 Hz, 3H).

### Synthesis 14

### (4,4-Difluorocyclohexyl)methanol (14)

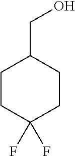

To a solution of ethyl 4-oxocyclohexanecarboxylate 13 (880 mg, 5.17 mmol) in CH₂Cl₂ (15 mL) was added diethylaminosulfur trifluoride (1.368 mL, 10.35 mmol) at 0° C. The resulting mixture was stirred for 24 h at 23° C. before it was quenched with a saturated solution of NaHCO₃ (20 mL). The resulting mixture was extracted with CH₂Cl₂ (4×10 mL), the combined organic layers were dried over Na₂SO₄ and concentrated in vacuo to afford the corresponding gem-difluoro- and vinyl fluoride as an inseparable mixture. The residue was passed through a short silica plug (EtOAc) and concentrated in vacuo prior to use.

To a CH₂Cl₂ solution of the mixture of gem-difluoro- and vinyl fluoride at 23° C. was added 3-chloroperbenzoic acid (1.158 g, 5.17 mmol, 77% in H₂O). The resulting reaction mixture was stirred for 16 h at 23° C. before it was quenched with a saturated solution of Na₂SO₃ (10 mL). The resulting mixture was extracted with CH₂Cl₂ (4×10 mL), the combined organic layers were dried over Na₂SO₄ and concentrated in vacuo to give a crude mixture of gem-difluoride and fluoroepoxide. This mixture which was re-dissolved in THF (10 mL) and lithium aluminium hydride (392 mg, 10.33 mmol) was added at 0° C. The resulting reaction mixture was stirred for 4 h at 0° C. before it was diluted with Et₂O (10 mL) and slowly quenched with H₂O (0.5 mL) followed by NaOH (0.5 mL, 15% aq.). The mixture was stirred for further 30 min at 23° C. before it was filtered. The filtration cake was washed with Et₂O (2×10 mL) and the combined filtrate concentrated in vacuo. The residue was purified by flash column chromatography (silica gel, hexanes:EtOAc 3:1→1:1) to afford compound 14 (480 mg, 62% for 3 steps) as a pale yellow oil.

¹H NMR (400 MHz, CDCl₃) δ=3.47 (d, J=8.0 Hz, 2H), 2.25-1.97 (m, 3H), 1.86-1.47 (m, 5H), 1.33-1.15 (m, 2H); ¹⁹F NMR (376 MHz, CDCl₃) δ=−91.35 (d, J=235.3 Hz, 1F), −102.01 (dtt, J=235.3, 32.4, 10.0 Hz, 1F).

### Synthesis 15

### 4,4-Difluorocyclohexanecarbaldehyde (15)

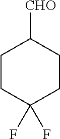

To a solution of DMSO (710 μL, 10.0 mmol) in CH₂Cl₂ (10 mL) was added oxalyl chloride (425 μL, 5.02 mmol) at −78° C. The resulting mixture was stirred for a further 20 min at −78° C. before it was cannulated with a CH₂Cl₂ (2 mL) solution of 4,4-difluorocyclohexylmethanol 14 (500 mg, 3.30 mmol) at −78° C. The reaction mixture was stirred for 30 min at −78° C. prior to the addition of Et₃N (2.33 mL, 16.7 mmol). The mixture was warmed to 0° C. and stirred for 30 min before it was diluted with CH₂Cl₂ (10 mL) and quenched with H₂O (10 mL). The layers were separated and the aqueous layer was extracted with CH₂Cl₂ (2×5 mL). The combined organic layers were dried over Na₂SO₄ and concentrated in vacuo. The residue was purified by flash column chromatography (silica gel, hexanes:EtOAc 4:1) to give compound 15 (315 mg, 64%) as a pale yellow oil.

¹H NMR (400 MHz, CDCl₃) δ=9.64 (s, 1H), 2.40-2.26 (m, 1H), 2.11-1.91 (m, 3H), 1.88-1.70 (m, 3H); ¹⁹F NMR (376 MHz, CDCl₃) δ=−94.9 (d, J=237.8 Hz, 1F), −98.9 (d, J=237.8 Hz, 1F).

### Synthesis 16

### Cyclohexyl(4,4-difluorocyclohexyl)methanol (5)

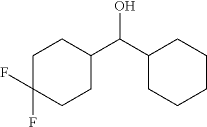 [SMILES: OC(C1CCCCC1)C1CCC(F)(F)CC1]

To a solution of 4,4-difluorocyclohexanecarboxaldehyde 15 (290 mg 1.96 mmol) in THF (10 mL) was added cyclohexylmagnesium chloride (2.5 mL, 2.5 mmol, 1.0 M in MeTHF) at 0° C. The resulting solution was stirred at 0° C. for 1 h before it was quenched with a saturated solution of NH₄Cl (10 mL). Following extraction with Et₂O (3×20 mL), the combined organic extracts were dried over Na₂SO₄ and evaporated in vacuo to give crude alcohol 5, which was used as described in synthesis 6 without further purification.

### Synthesis 17

### (4,4-Difluorocyclohexyl)(1,4-dioxaspiro[4.5]dec-7-en-8-yl)methanol (17)

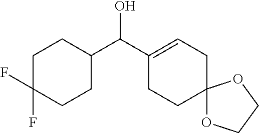 [SMILES: OC(C1=CCC2(CC1)OCCO2)C1CCC(F)(F)CC1]

To a solution of 2,4,6-triisopropyl-N-(1,4-dioxaspiro[4.5]decan-8-ylidene)benzenesulfonohydrazide (prepared according to the method reported by Hu et al., 2006) (655 mg, 1.50 mmol) in THF (10 mL) was added a solution of n-BuLi (2.20 mL, 3.30 mmol, 1.5 M in hexanes) at −78° C. The resulting red solution was stirred for 30 min prior to warming to 0° C. for 5 min, after which the solution was cooled to −78° C. and a solution of 4,4-difluorocyclohexanecarboxaldehyde 15 (222 mg, 1.50 mmol) in THF (2 mL) was added. The reaction mixture was stirred at −78° C. for 2 h before quenching with a saturated solution of NH₄Cl (20 mL). Following extraction with Et₂O (3×20 mL), the combined organic extracts were dried over Na₂SO₄ and evaporated in vacuo to give a yellow residue which was purified by flash chromatography (silica gel, hexanes:EtOAc 2:1) to furnish alcohol 17 (371 mg, 86%) as a pale yellow oil.

¹H NMR (400 MHz, CDCl₃) δ=5.58-5.52 (m, 1H), 3.97 (s, 4H), 3.73 (d, J=8.1 Hz, 1H), 2.40-2.24 (m, 3H), 2.17-2.01 (m, 3H), 1.81-1.57 (m, 6H), 1.57-1.44 (m, 3H); ¹⁹F NMR (376 MHz, CDCl₃) δ=−91.8 (d, J=234.5 Hz, 1F), −102.2 (dt, J=234.5, 32.8 Hz, 1F).

### Synthesis 18

### (4,4-Difluorocyclohexyl)(1,4-dioxaspiro[4.5]dec-7-en-8-yl)methyl benzoate (18)

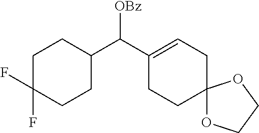

To a solution of alcohol 17 (300 mg, 1.04 mmol), 4-(dimethylamino)pyridine (12.0 mg, 0.1 mmol) and pyridine (255 μL, 3.15 mmol) in CH₂Cl₂ (2 mL) was added benzoyl chloride (180 μL, 1.55 mmol) at 0° C. The resulting reaction mixture was warmed to 23° C. and stirred for 16 h before it was quenched with a saturated solution of NaHCO₃ (10 mL) and diluted with Et₂O (30 mL). The layers were separated and the organic layer was washed with a solution of HCl (1 M, 2×5 mL) followed by brine (2×5 mL). The combined organic extracts were dried over Na₂SO₄ and evaporated in vacuo to give a yellow residue which was purified by flash chromatography (silica gel, hexanes:EtOAc 4:1) to give compound 18 (363 mg, 89%) as a colourless oil.

¹H NMR (400 MHz, CDCl₃) δ=8.04 (dd, J=8.6, 1.2 Hz, 2H), 7.59-7.53 (m, 1H), 7.44 (dd, J=8.6, 4.7 Hz, 2H), 5.72-5.66 (m, 1H), 5.26 (d, J=8.3 Hz, 1H), 4.03-3.87 (m, 4H), 2.39-2.18 (m, 4H), 2.17-2.05 (m, 2H), 1.95-1.59 (m, 7H), 1.49-1.32 (m, 2H); ¹⁹F NMR (376 MHz, CDCl₃) δ=−92.2 (d, J=236.6 Hz, 1F), −102.1 (d, J=236.6 Hz, 1F).

### Synthesis 19

### (4,4-Difluorocyclohexyl)(4-oxocyclohexyl)methyl benzoate (19)

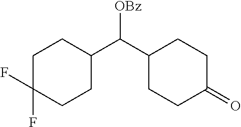

Palladium on activated charcoal (20 mg, 10% wt/wt) was suspended in a solution of benzoate 18 (200 mg) in MeOH (10 mL) at 23° C. under an atmosphere of hydrogen (1 atm) for 16 h, before it was filtered through a pad of Celite® and the filtrate concentrated in vacuo. The residue was dissolved in THF (5 mL) at 23° C. and a solution of HCl (2 N, 5 mL) was added. The resulting mixture was stirred for 6 h before it was quenched with a saturated solution of NaHCO₃ (15 mL) and diluted with CH₂Cl₂ (10 mL). The layers were separated, and the aqueous layer was extracted with CH₂Cl₂ (4×5 mL). The combined organic layers were dried over Na₂SO₄ and concentrated in vacuo. The residue was purified by flash chromatography (silica gel, hexanes:EtOAc 2:1) to give ketone 19 (139 mg, 78% for 2 steps) as a colourless oil.

¹H NMR (400 MHz, CDCl₃) δ=8.06-7.98 (m, 2H), 7.63-7.56 (m, 1H), 7.52-7.42 (m, 2H), 5.12 (t, J=5.8, 1 H), 2.49-2.26 (m, 4H), 2.24-1.99 (m, 5H), 1.91-1.46 (m, 9H); ¹⁹F NMR (376 MHz, CDCl₃) δ=−91.9 (d, J=235.8 Hz, 1F), −102.5 (dt, J=235.8, 32.4 Hz, 1F).

### Synthesis 20

### Bis(4,4-difluorocyclohexyl)methanol (20)

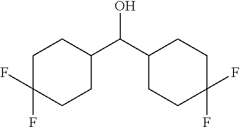 [SMILES: OC(C1CCC(F)(F)CC1)C1CCC(F)(F)CC1]

To a solution of ketone 19 (64 mg, 0.183 mmol) in CH₂Cl₂ (2 mL) was added diethylaminosulfur trifluoride (53 μL, 4.02 mmol) at 0° C. The resulting mixture was stirred for 24 h at 23° C. before it was quenched with a saturated solution of NaHCO₃ (5 mL). The resulting mixture was extracted with CH₂Cl₂ (3×10 mL), the combined organic layers were dried over Na₂SO₄ and concentrated in vacuo to afford the corresponding gem-difluoro and vinyl fluoride as an inseparable mixture. The residue was passed through a short silica plug (EtOAc) and concentrated in vacuo prior to use.

To a CH₂Cl₂ solution of the mixture of the gem-difluoro and vinyl fluoride at 23° C. was added 3-chloroperbenzoic acid (41 mg, 0.309 mmol, 77% in H₂O). The resulting reaction mixture was stirred for 16 h at 23° C. before quenching with a saturated solution of Na₂SO₃ (5 mL). The resulting mixture was extracted with CH₂Cl₂ (3×10 mL), the combined organic layers were dried over Na₂SO₄ and concentrated in vacuo to give a crude mixture of gem-difluoride and fluoroepoxide. This mixture was re-suspended in THF-MeOH (10 mL, 4:1, v/v) and a solution of KOH (1 mL, 10% aq.) was added at 23° C. The mixture was warmed to 60° C. for 6 h before it was diluted with H₂O (20 mL). The resulting mixture was extracted with CH₂Cl₂ (3×10 mL), the combined organic layers were dried over Na₂SO₄ and concentrated in vacuo. The residue was purified by flash column chromatography (silica gel, hexanes:EtOAc 5:1→1:1) to afford alcohol 20 (23 mg, 48% for 3 steps) as a colourless oil.

¹H NMR (400 MHz, CDCl₃) δ=4.70 (br s, 1H), 3.29-3.22 (m, 1H), 2.23-2.06 (m, 4H), 1.95-1.84 (m, 2H), 1.82-1.58 (m, 4H), 1.58-1.33 (m, 6H), 1.31-1.20 (m, 2H); ¹³C NMR (101 MHz, CDCl₃) δ=123.6 (t, J=242.0 Hz), 77.7 (t, J=2.6 Hz), 38.5 (d, J=2.6 Hz), 33.6 (d, J=25.4 Hz), 33.1 (d, J=25.4 Hz), 26.0 (d, J=10.6 Hz), 23.3 (d, J=10.6 Hz); ¹⁹F NMR (376 MHz, CDCl₃) δ=−91.5 (d, J=236.7 Hz, 2F), −102.8 (dtt, J=236.7, 33.8, 10.6 Hz, 2F).

### Synthesis 21

### Bis(4,4-difluorocyclohexyl)methanone (21)

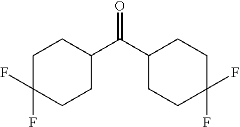 [SMILES: O=C(C1CCC(F)(F)CC1)C1CCC(F)(F)CC1]

To a solution of alcohol 20 (20 mg, 0.075 mmol) in CH₂Cl₂ (2 mL) was added Dess-Martin periodinane (47 mg, 0.112 mmol) at 23° C. The reaction mixture was stirred for 16 h before it was quenched with a saturated solution of Na₂SO₃ (5 mL). The resulting mixture was extracted with CH₂Cl₂ (2×10 mL), the combined organic layers were dried over Na₂SO₄ and concentrated in vacuo. The residue was purified by flash column chromatography (silica gel, hexanes:EtOAc 5:1) to give ketone 21 (14 mg, 71%) as an amorphous white solid.

¹H NMR (400 MHz, CDCl₃) δ=2.66-2.56 (m, 2H), 2.24-2.11 (m, 4H), 1.95-1.84 (m, 4H), 1.84-1.69 (m, 8H); ¹³C NMR (101 MHz, CDCl₃) δ=213.1, 122.5 (t, J=240.5 Hz), 46.3, 32.9 (d, J=24.1 Hz), 32.6 (d, J=24.1 Hz), 25.0, 24.8; ¹⁹F NMR (376 MHz, CDCl₃) δ=−93.2 (d, J=238.9 Hz, 2F), −100.8 (d, J=238.9 Hz, 2F).

### Synthesis 22

### 2-(2,2-Bis(4,4-Difluorocyclohexyl)ethyl)piperidine (22)

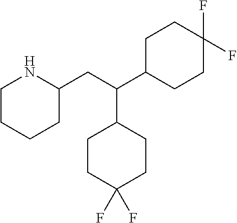 [SMILES: FC1(F)CCC(C(CC2CCCCN2)C2CCC(F)(F)CC2)CC1]

To a solution of 2-picoline (7 μL, 0.071 mmol) in THF (2 mL) was added a solution of n-BuLi (45 μL, 0.068 mmol, 1.5 M in hexanes) at −78° C. The resulting brick-red solution was warmed to −20° C. and stirred for 30 min before it was cooled to −78° C., affording a lithiated 2-picoline solution. A THF (1 mL) solution of ketone 21 (14 mg, 0.062 mmol) was cannulated to the lithiated 2-picoline solution at −78° C. and stirred for 30 min prior to the addition of SOCl₂ (6 μL, 0.078 mmol). The reaction mixture was warmed to 23° C. and stirred for 1 h before it was quenched with a saturated solution of NaHCO₃ (5 mL). The resulting mixture was extracted with CH₂Cl₂ (3×10 mL), the combined organic layers were dried over Na₂SO₄ and concentrated in vacuo. The residue was passed through a short silica plug (EtOAc) and concentrated in vacuo prior to use.

Palladium on activated charcoal (4 mg, 20% wt/wt) was suspended in a solution of the above crude residue in HOAc (5 mL) with at 50° C. under an atmosphere of hydrogen (1 atm) for 60 h, before it was filtered through a pad of Celite®. The filtrate was diluted with H₂O (20 mL) and the pH value of the aqueous solution was adjusted to 10 with NaHCO₃ prior to the extraction of the solution with CH₂Cl₂ (5×10 mL). The combined organic layers were dried over Na₂SO₄ and concentrated in vacuo. The residue was purified by flash column chromatography (silica gel, NH₄OH_{aq}:MeOH:CH₂Cl₂=2:10:88, v/v) to obtain compound 22 (12 mg, 65% for 2 steps) as a pale yellow oil.

¹H NMR (400 MHz, CDCl₃) δ=3.15 (d, J=12.0 Hz, 1H), 2.65 (t, J=12.0 Hz, 1H), 2.50 (s, 1H), 2.22-2.02 (m, 4H), 1.87-1.54 (m, 11H), 1.54-1.02 (m, 12H); ¹³C NMR (101 MHz, CDCl₃) δ=123.4 (t, J=236.6 Hz), 123.3 (t, J=236.6 Hz), 56.6, 46.6, 42.6, 38.5, 38.0, 34.2 (d, J=3.7 Hz), 34.1 (d, J=3.7 Hz), 34.0 (d, J=3.7 Hz), 33.8 (d, J=3.7 Hz), 33.7 (d, J=3.7 Hz), 33.5 (d, J=3.7 Hz), 29.7, 27.6, 27.5, 27.3, 27.2, 26.0, 25.9, 25.3, 25.2, 24.3; ¹⁹F NMR (376 MHz, CDCl₃) δ=−91.2 (dd, J=236.0, 30.0 Hz, 2F), −102.6 (dm, J=236.0 Hz, 2F); m/z (ESI) 350.2 [M+H]⁺.

### Synthesis 23

### 1,4-Dioxaspiro[4.5]dec-7-ene-8-carbaldehyde (23)

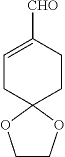 [SMILES: O=CC1=CCC2(CC1)OCCO2]

To a solution of 2,4,6-triisopropyl-N-(1,4-dioxaspiro[4.5]decan-8-ylidene)benzenesulfonohydrazide (prepared according to the method reported by Hu et al., 2006) (1.24 g, 2.84 mmol) in THF (20 mL) was added a solution of n-BuLi (2.50 mL, 6.25 mmol, 2.5 M in hexanes) at −78° C. The resulting red solution was stirred at −78° C. for 30 min and then warmed to 0° C. for 5 min, after which the solution was cooled to −78° C. and N,N-dimethylformamide (1.10 mL, 14.3 mmol) was added. The reaction mixture was stirred at −20° C. for 3 h before it was quenched with a saturated solution of NH₄Cl (20 mL). Following extraction with Et₂O (3×20 mL), the combined organic extracts were dried over Na₂SO₄ and evaporated in vacuo to give a yellow residue which was purified by flash chromatography (silica gel, hexanes:EtOAc 4:1) to give compound 23 (334 mg, 70%) as a colourless oil.

¹H NMR (400 MHz, CDCl₃) δ=9.48 (s, 1H), 6.75-6.66 (m, 1H), 4.02 (s, 4H), 2.64-2.54 (m, 2H), 2.46 (ddt, J=8.7, 6.5, 2.1 Hz, 2H), 1.81 (t, J=6.5 Hz, 2H).

### Synthesis 24

### 1,4-Dioxaspiro[4.5]decane-8-carbaldehyde (24)

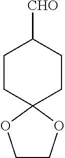 [SMILES: O=CC1CCC2(CC1)OCCO2]

10% palladium on activated charcoal (35 mg) was suspended in a solution of α,β-unsaturated aldehyde 23 (330 mg) in THF (10 mL) at 23° C. under an atmosphere of hydrogen (1 atm) for 4 h, before it was filtered through a pad of Celite® and the filtrate concentrated in vacuo. The residue was passed through a short plug of silica gel (EtOAc) to afford aldehyde 24 (281 mg, 85%) as a colourless oil.

¹H NMR (400 MHz, CDCl₃) δ=9.67 (d, J=1.3 Hz, 1H), 3.97 (s, 4H), 2.35-2.21 (m, 1H), 2.02-1.91 (m, 2H), 1.85-1.72 (m, 4H), 1.68-1.51 (m, 2H).

### Synthesis 25

### Cyclohexyl(1,4-dioxaspiro[4.5]decan-8-yl)methanol (25)

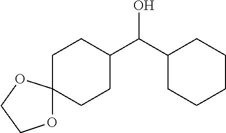 [SMILES: OC(C1CCCCC1)C1CCC2(CC1)OCCO2]

To a solution of aldehyde 24 (300 mg, 1.763 mmol) in THF (10 mL) was added cyclohexylmagnesium chloride (2.10 mL, 2.10 mmol, 1.0 M in MeTHF) at 0° C. The resulting solution was stirred at 0° C. for 1 h before quenching with a saturated solution of NH₄Cl (10 mL). Following extraction with Et₂O (3×20 mL), the combined organic extracts were dried over Na₂SO₄ and evaporated in vacuo to give a yellow residue which was purified by flash column chromatography (silica gel; hexane:EtOAc, 1:1) to give alcohol 25 (350 mg, 78%) as a pale yellow oil.

¹H NMR (400 MHz, CDCl₃) δ=3.94 (s, 4H), 3.13 (dd, J=5.3, 5.1 Hz, 1H), 1.95-1.72 (m, 6H), 1.72-1.37 (m, 8H), 1.37-0.99 (m, 7H); ¹³C NMR (101 MHz, CDCl₃) δ=108.9, 79.5, 64.23, 64.22, 40.2, 38.7, 34.6, 34.3, 30.1, 27.0, 26.9, 26.51, 26.48, 26.1, 24.8.

### Synthesis 26

### 4-(Cyclohexyl(hydroxy)methyl)cyclohexanone (26)

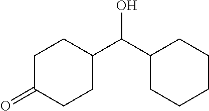 [SMILES: O=C1CCC(C(O)C2CCCCC2)CC1]

To a THF solution (6 mL) of alcohol 25 (340 mg, 1.34 mmol), was added an aqueous solution of HCl (2 M, 2 mL) at 23° C. The resulting mixture was stirred at 50° C. for 6 h before it was quenched with a saturated solution of NaHCO₃ (15 mL) and diluted with CH₂Cl₂ (10 mL). The layers were separated, and the aqueous layer was extracted with CH₂Cl₂ (4×5 mL). The combined organic layers were dried over Na₂SO₄ and concentrated in vacuo. The residue was purified by flash column chromatography (silica gel, hexanes:EtOAc 1:1) to give ketone 26 (266 mg, 95%) as a colourless oil.

¹H NMR (400 MHz, CDCl₃) δ=3.27-3.19 (m, 1H), 2.65 (br s, 1H, OH), 2.49-2.26 (m, 3H), 2.26-2.13 (m, 2H), 1.99-1.75 (m, 4H), 1.75-1.39 (m, 5H), 1.37-1.00 (m, 6H); ¹³C NMR (101 MHz, CDCl₃) δ=212.1, 78.9, 40.8, 40.53, 40.49, 38.2, 30.0, 29.6, 27.2, 27.0, 26.41, 26.36, 26.1.

### Synthesis 27

### N-(4-(Cyclohexyl(triethylsilyloxy)methyl)cyclohexylidene)-2,4,6-triisopropylbenzenesulfonohydrazide (27)

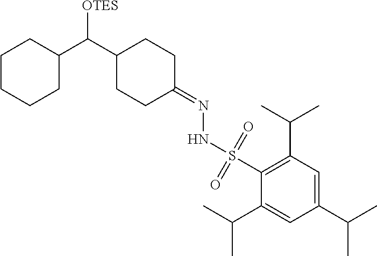

To a CH₂Cl₂ solution (5 mL) of ketone 26 (250 mg, 1.19 mmol) was added imidazole (162 mg, 2.38 mmol) and triethylchlorosilane (250 μL, 1.49 mmol) at 0° C. The resulting mixture was stirred at 0° C. for 2 h before it was quenched with a saturated solution of NaHCO₃ (5 mL) and diluted with CH₂Cl₂ (10 mL). The layers were separated, and the aqueous layer was extracted with CH₂Cl₂ (2×5 mL). The combined organic layers were dried over Na₂SO₄ and concentrated in vacuo. The residue was passed through a plug of silica gel (EtOAc) and concentrated in vacuo to give the triethylsilyl-protected alcohol, which was used without further purification.

The protected alcohol which was re-dissolved in THF (5 mL) and 2,4,6-triisopropylbenzenesulfonohydrazide (355 mg, 1.19 mmol) was added at 0° C. The resulting mixture was stirred at 0° C. and allowed to warm to 23° C. for 16 h before it was concentrated in vacuo to give hydrazone 27 as a white solid.

### Synthesis 28

### (Cyclohexyl(4-fluorocyclohex-3-enyl)methoxy)triethylsilane (28)

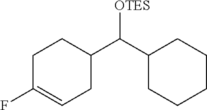

To a solution of hydrazone 27 (640 mg, 1.06 mmol) in THF (10 mL) was added a solution of n-BuLi (990 μL, 2.38 mmol, 2.4 M in hexanes) at −78° C. The resulting orange-red solution was stirred at −78° C. for 30 min prior to warming to 0° C. for 2 min, after which the solution was cooled to −78° C. and a THF solution (2 mL) of N-fluorobenzenesulfonimide (535.0 mg, 1.70 mmol) was added dropwise. The resulting mixture was stirred at −78° C. for 4 h and allowed to warm to 23° C. for 12 h prior to quenching with a saturated solution of NH₄Cl (20 mL). Following extraction with Et₂O (3×10 mL), the combined organic extracts were dried over Na₂SO₄ and evaporated in vacuo to give an inseparable 2.8:1 mixture (by ¹H NMR) of vinyl fluoride 28 and the protonated by-product, (cyclohex-3-enyl(cyclohexyl)methoxy)triethylsilane, as a yellow oil.

### Synthesis 29

### Cyclohexyl(4-fluorocyclohexyl)methanol (29)

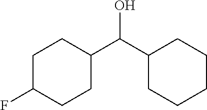 [SMILES: OC(C1CCCCC1)C1CCC(F)CC1]

To a CH₂Cl₂-MeOH (10:1, v/v) solution (5 mL) of the mixture of vinyl fluoride 28 and (cyclohex-3-enyl(cyclohexyl)methoxy)triethylsilane was added p-toluenesulfonic acid (50 mg) at 0° C. The reaction mixture was stirred at 0° C. for 1 h before it was quenched with a saturated solution of NaHCO₃ (5 mL) and diluted with CH₂Cl₂ (10 mL). The layers were separated and the aqueous layer was extracted with CH₂Cl₂ (2×5 mL). The combined organic layers were dried over Na₂SO₄ and concentrated in vacuo. 10% palladium on activated charcoal (12 mg) was suspended in a solution of the residue in THF at 23° C., under an atmosphere of hydrogen (1 atm) for 48 h, before it was filtered through a pad of Celite® and the filtrate concentrated in vacuo. The residue was purified by flash column chromatography (silica gel, hexanes:EtOAc 10:1) to give alcohol 29 (75 mg, 29% for 5 steps, a mixture of cis- and trans-isomers) as a colourless oil.

### Synthesis 30

### Cyclohexyl(4-fluorocyclohexyl)methanol (30)

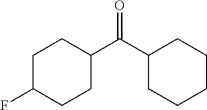 [SMILES: O=C(C1CCCCC1)C1CCC(F)CC1]

To a solution of alcohol 29 (42 mg, 0.196 mmol) in CH₂Cl₂ (5 mL) was added Dess-Martin periodinane (104 mg, 0.250 mmol) at 23° C. The reaction mixture was stirred for 16 h before it was quenched with saturated solution of Na₂SO₃ (5 mL). The resulting mixture was extracted with CH₂Cl₂ (2×10 mL), the combined organic layers were dried over Na₂SO₄ and concentrated in vacuo. The residue was purified by flash column chromatography (silica gel, hexanes:EtOAc 10:1) to give ketone 30 (33.7 mg, 81%, ca. 1.4:1 mixture of cis- and trans-isomers) as an amorphous white solid.

¹H NMR (400 MHz, CDCl₃) δ=4.83 (dm, J=48.3 Hz, 0.42; H), 4.51 (dm, J=49.5 Hz, 0.58; H), 2.59-2.44 (m, 2H), 2.21-2.00 (m, 2H), 1.94-1.73 (m, 6H), 1.72-1.61 (m, 2H), 1.61-1.43 (m, 3H), 1.43-1.16 (m, 5H); ¹³C NMR (101 MHz, CDCl₃) δ=216.1 (d, J=2.5 Hz), 215.9, 91.3 (d, J=172.1 Hz), 88.0 (d, J=168.2 Hz), 49.6, 48.8, 47.8, 47.11, 47.09, 31.6 (d, J=18.6 Hz), 28.7, 28.5, 26.0, 25.84, 25.80, 25.71, 25.66, 22.63, 22.61; ¹⁹F NMR (376 MHz, CDCl₃) δ=−171.1 (d, J=49.5 Hz, 0.58F), −184.2 (dm, J=48.3 Hz, 0.42F).

### Synthesis 31

### (E) and (2)-2-(2-cyclohexyl-2-(4-fluorocyclohexyl)vinyl)pyridine (31) and 2-(2-Cyclohexyl-2-(4-fluorocyclohexylidene)ethyl)pyridine/2-(2-Cyclohexylidene-2-(4-fluorocyclohexyl)ethyl)pyridine (32)

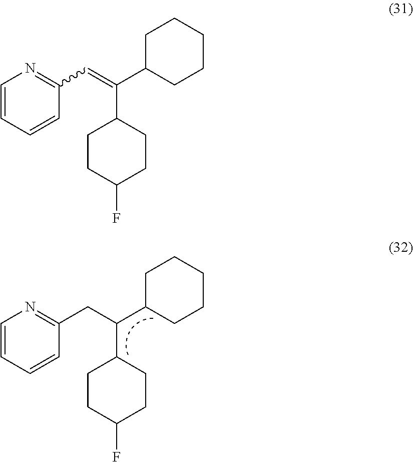 [SMILES: FC1CCC(C(=Cc2ccccn2)C2CCCCC2)CC1 ; FC1CCC(C(Cc2ccccn2)C2CCCCC2)CC1]

To a solution of 2-picoline (20 μL, 0.202 mmol) in THF (2 mL) was added a solution of n-BuLi (84 μL, 0.202 mmol, 2.4 M in hexanes) at −78° C. The resulting brick-red solution was warmed to −20° C. and stirred for 30 min before it was cooled to −78° C., affording a lithiated 2-picoline solution. A solution of ketone 30 (32 mg, 0.151 mmol) in THF (1 mL) was cannulated to the lithiated 2-picoline solution at −78° C. and stirred for 30 min prior to the addition of SOCl₂ (18 μL, 0.233 mmol). The reaction mixture was warmed to 23° C. and stirred for 1 h before quenching with a saturated solution of NaHCO₃ (5 mL). The resulting mixture was extracted with CH₂Cl₂ (3×10 mL), the combined organic layers were dried over Na₂SO₄ and concentrated in vacuo to give a mixture of compounds 31 and 32. The residue was passed through a short silica plug (EtOAc) and concentrated in vacuo prior to further use.

### Synthesis 32

### 2-(2-Cyclohexyl-2-(4-fluorocyclohexyl)ethyl)piperidine (33)

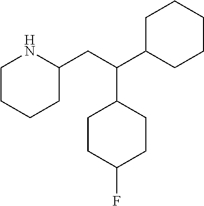 [SMILES: FC1CCC(C(CC2CCCCN2)C2CCCCC2)CC1]

10% palladium on activated charcoal (5 mg) was suspended in a solution of the crude mixture of 31 and 32 in HOAc (5 mL) at 50° C. under an atmosphere of hydrogen (1 atm) for 24 h. After which an equal amount of 10% palladium on activated charcoal was added to the reaction mixture and stirred for further 48 h at 50° C. before it was filtered through a pad of Celite®. The filtrate was diluted with H₂O (20 mL) and the pH value of the aqueous solution was adjusted to 10 with NaHCO₃ prior to extraction of the solution with CH₂Cl₂ (5×10 mL). The combined organic layers were dried over Na₂SO₄ and concentrated in vacuo. The residue was purified by flash column chromatography (silica gel, NH₄OH_{aq}:MeOH:CH₂Cl₂=2:10:88, v/v) to obtain compound 33 (28 mg, 64% for 2 steps, a mixture of diastereomers) as a pale yellow oil.

The spectroscopic data of major diastereomers is described as follows: ¹H NMR (400 MHz, CDCl₃) δ=4.83 (dm, J=48.5 Hz, 0.38; H), 4.44 (dm, J=49.5 Hz, 0.62; H), 3.66 (br s, 1H, NH), 3.23-3.10 (m, 1H), 2.75-2.60 (m, 1H), 2.60-2.43 (m, 1H), 2.22-1.93 (m, 4H), 1.89-1.57 (m, 12H), 1.55-0.86 (m, 13H); ¹³C NMR (101 MHz, CDCl₃) δ=92.7 (d, J=172.3 Hz), 89.0 (d, J=166.5 Hz), 88.9 (d, J=166.5 Hz), 56.9, 56.7, 46.8, 46.6, 44.8, 44.7, 44.1, 40.3, 40.1, 39.9, 39.5, 39.3, 39.0, 38.9, 38.7, 38.4, 33.1, 32.9, 32.7, 32.0, 31.9, 31.8, 31.7, 31.6, 31.4, 31.2, 31.13, 31.09, 31.0, 29.9, 29.7, 27.1, 27.0, 26.8, 26.7, 26.6, 25.4, 24.5; ¹⁹F NMR (376 MHz, CDCl₃) δ=−168.8 (dm, J=49.5 Hz, 0.62F), −184.5-−185.1 (m, 0.38F); m/z (ESI) 296.3 [M+H]⁺.

### Synthesis 33

### 1,4-Dioxaspiro[4.5]dec-7-en-8-yl(1,4-dioxaspiro[4.5]decan-8-yl)methanol (34)

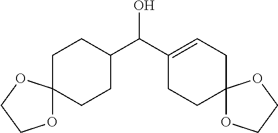 [SMILES: OC(C1=CCC2(CC1)OCCO2)C1CCC2(CC1)OCCO2]

To a solution of 2,4,6-triisopropyl-N-(1,4-dioxaspiro[4.5]decan-8-ylidene)benzenesulfonohydrazide (225 mg, 0.515 mmol) in THF (10 mL) was added a solution of n-BuLi (0.515 mL, 1.288 mmol, 2.5 M in hexanes) at −78° C. The resulting red solution was stirred at −78° C. for 30 min and then warmed to 0° C. for 2 min, after which the solution was cooled to −78° C. and the α,β-unsaturated aldehyde 23 (70 mg, 0.411 mmol) in THF (2 mL) was added at −78° C. The reaction mixture was stirred at −78° C. for 1 h before it was quenched with a saturated solution of NH₄Cl (20 mL). Following extraction with Et₂O (3×20 mL), the combined organic extracts were dried over Na₂SO₄ and evaporated in vacuo to give a yellow residue which was purified by flash chromatography (silica gel, hexanes:EtOAc 1:1) to give allylic alcohol 34 (84 mg, 66%) as a colourless oil.

¹H NMR (400 MHz, CDCl₃) δ=5.54-5.49 (m, 1H), 3.97 (s, 4H), 3.94-3.91 (m, 4H), 3.72 (d, J=8.2 Hz, 1H, OH), 2.40-2.25 (m, 3H), 2.18-1.98 (m, 2H), 1.85-1.69 (m, 5H), 1.58-1.44 (m, 4H), 1.38-1.15 (m, 2H); ¹³C NMR (101 MHz, CDCl₃) δ=138.8, 121.6, 108.9, 108.1, 80.4, 64.40, 64.36, 64.2, 39.4, 35.6, 34.3, 34.1, 30.9, 26.7, 26.5, 22.3.

### Synthesis 34

### Di1,4-dioxaspiro[4.5]decan-8-ylmethanol (35)

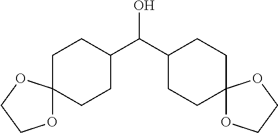 [SMILES: OC(C1CCC2(CC1)OCCO2)C1CCC2(CC1)OCCO2]

Palladium on activated charcoal (6 mg, 10% wt/wt) was suspended in a solution of allylic alcohol 34 (63 mg) in MeOH (10 mL) at 23° C. under an atmosphere of hydrogen (1 atm) for 16 h, before it was filtered through a pad of Celite® and the filtrate was cooled to 0° C. followed by addition of NaBH₄ (12 mg, 0.317 mmol). The reaction mixture was stirred at 0° C. for 1 h before it was quenched with acetone (1 mL), a saturated solution of NaHCO₃ (10 mL) and diluted with CH₂Cl₂ (10 mL). The layers were separated, and the aqueous layer was extracted with CH₂Cl₂ (4×5 mL). The combined organic layers were dried over Na₂SO₄ and concentrated in vacuo to give a white residue which was purified by flash chromatography (silica gel, hexanes:EtOAc 1:2) to give alcohol 35 (54 mg, 85% for 2 steps) as a colourless solid.

¹H NMR (400 MHz, CDCl₃) δ=3.95 (s, 8H), 3.20 (s, 1H), 1.91-1.74 (m, 7H), 1.64-1.36 (m, 9H), 1.34-1.18 (m, 3H); ¹³C NMR (101 MHz, CDCl₃) δ=108.8, 78.6, 64.2, 39.0, 34.6, 34.3, 27.1, 24.4.

### Synthesis 35

### 4,4′-(Hydroxymethylene)dicyclohexanone (36)

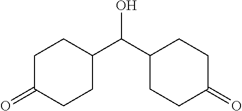 [SMILES: O=C1CCC(C(O)C2CCC(=O)CC2)CC1]

To a THF solution (5 mL) of alcohol 35 (50 mg, 0.16 mmol), was added an aqueous solution of HCl (2 N, 1 mL) at 23° C. The resulting mixture was stirred at 50° C. for 6 h before it was quenched with a saturated solution of NaHCO₃ (10 mL) and diluted with CH₂Cl₂ (10 mL). The layers were separated, and the aqueous layer was extracted with CH₂Cl₂ (4×5 mL). The combined organic layers were dried over Na₂SO₄ and concentrated in vacuo. The residue was purified by flash column chromatography (silica gel, hexanes:EtOAc 1:2) to give ketone 36 (34 mg, 94%) as an amorphous white solid.

¹H NMR (400 MHz, CDCl₃) δ=4.70 (br s, 1H, OH), 3.42 (s, 1H), 2.54-2.31 (m, 8H), 2.30-2.18 (m, 2H), 2.06-1.90 (m, 4H), 1.82-1.48 (m, 4H); ¹³C NMR (101 MHz, CDCl₃) δ=211.3, 77.5, 40.6, 40.4, 38.8, 29.6, 26.9.

### Synthesis 36

### N,N′-(4,4′-((Triethylsilyloxy)methylene)bis(cyclohexan-4-yl-1-ylidene))bis(2,4,6-triisopropylbenzenesulfonohydrazide) (37)

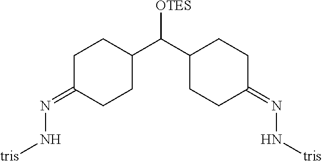

To a CH₂Cl₂ solution (5 mL) of ketone 36 (30 mg, 0.134 mmol) was added imidazole (18 mg, 0.264 mmol) and triethylchlorosilane (27 μL, 0.161 mmol) at 0° C. The resulting mixture was stirred at 0° C. for 2 h before it was quenched with a saturated solution of NaHCO₃ (5 mL) and diluted with CH₂Cl₂ (10 mL). The layers were separated, and the aqueous layer was extracted with CH₂Cl₂ (2×5 mL). The combined organic layers were dried over Na₂SO₄ and concentrated in vacuo. The residue was passed through a plug of silica gel (EtOAc) and concentrated in vacuo to give the triethylsilyl-protected alcohol, which was used without further purification. The protected alcohol which was re-dissolved in THF (5 mL) and 2,4,6-triisopropyl benzenesulphonylhydrazine (87 mg, 0.292 mmol) was added at 0° C. The resulting mixture was stirred at 0° C. and allowed to warm to 23° C. for 16 h before it was concentrated in vacuo to give hydrazone 37 (90 mg, 75% for two steps) as white solid.

m/z (ESI) 899.5 [M+H]⁺.

### Synthesis 37

### (Bis(4-fluorocyclohex-3-enyl)methoxy)triethylsilane (38)

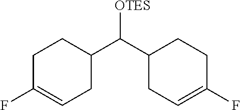

To a solution of hydrazone 37 (90 mg, 1.06 mmol) in THF (5 mL) was added a solution of n-BuLi (270 μL, 0.608 mmol, 2.25 M in hexanes) at −78° C. The resulting orange-red solution was stirred at −78° C. for 30 min prior to warming to 0° C. for 2 min, after which the solution was cooled to −78° C. and a THF solution (2 mL) of N-fluorobenzenesulfonimide (126 mg, 0.4 mmol) was added dropwise. The resulting mixture was stirred at −78° C. for 4 h and allowed to warm to 23° C. for 12 h prior to quenching with a saturated solution of NH₄Cl (10 mL). Following extraction with Et₂O (3×10 mL), the combined organic extracts were dried over Na₂SO₄ and evaporated in vacuo to afford crude bis-vinyl fluoride 38 as a pale yellow oil.

### Synthesis 38

### Bis(4-fluorocyclohexyl)methanol (39)

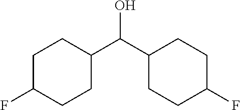 [SMILES: OC(C1CCC(F)CC1)C1CCC(F)CC1]

To a CH₂Cl₂-MeOH (10:1, v/v) solution (5 mL) of the crude bis-vinyl fluoride 38 was added p-toluenesulfonic acid (5 mg) at 0° C. The reaction mixture was stirred at 0° C. for 1 h before it was quenched with a saturated solution of NaHCO₃ (5 mL) and diluted with CH₂Cl₂ (10 mL). The layers were separated and the aqueous layer was extracted with CH₂Cl₂ (2×5 mL). The combined organic layers were dried over Na₂SO₄ and concentrated in vacuo. 10% palladium on activated charcoal (10 mg) was suspended in a solution of the residue in THF (5 mL) at 23° C., under an atmosphere of hydrogen (1 atm) for 48 h, before it was filtered through a pad of Celite® and the filtrate concentrated in vacuo. The residue was purified by flash column chromatography (silica gel, hexanes:EtOAc 3:1) to give alcohol 39 (6 mg, 26% for 3 steps, a mixture of cis- and trans-isomers) as a colourless oil.

¹H NMR (400 MHz, CDCl₃) δ=4.88 (dm, J=48.9 Hz, 1H), 4.70 (brs, 0.5; H, OH), 4.47 (dtt, J=48.9, 4.9, 1.5 Hz, 1H), 4.30 (brs, 0.5; H, OH), 3.00 (dd, J=14.5, 6.8 Hz, 1H), 2.22-2.05 (m, 4H), 2.00-1.87 (m, 2H), 1.76-1.64 (m, 2H), 1.54-1.39 (m, 4H), 1.37-1.12 (m, 6H); ¹⁹F NMR (376 MHz, CDCl₃) δ=−169.3 (dm, J=48.5 Hz, 0.5F), −169.6 (dm, J=48.5 Hz, 0.5F), −185.3 (m, 1F).

### Synthesis 39

### Bis(4-fluorocyclohexyl)methanone (40)

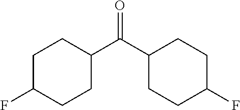 [SMILES: O=C(C1CCC(F)CC1)C1CCC(F)CC1]

To a solution of alcohol 39 (14 mg, 0.06 mmol) in CH₂Cl₂ (2 mL) was added Dess-Martin periodinane (38 mg, 0.09 mmol) at 23° C. The reaction mixture was stirred for 16 hours before it was quenched with a saturated solution of Na₂SO₃ (2 mL). The resulting mixture was extracted with CH₂Cl₂ (2×10 mL), combined organic layers were dried over Na₂SO₄ and concentrated in vacuo. The residue was purified by flash column chromatography (silica gel, hexanes:EtOAc 5:1) to give ketone 40 (10 mg, 72%) as a colourless oil.

¹H NMR (400 MHz, CDCl₃) δ=4.84 (dm, J=48.3 Hz, 1H), 4.52 (dm, J=48.3 Hz, 1H), 2.61-2.47 (m, 2H), 2.24-2.04 (m, 4H), 1.98-1.85 (m, 2H), 1.84-1.73 (m, 3H), 1.72-1.63 (m, 2H), 1.62-1.40 (m, 5H); ¹⁹F NMR (376 MHz, CDCl₃) δ=−172.0 (dm, J=48.3 Hz, 0.5F), −171.4 (dm, J=48.3 Hz, 0.5F), −184.0-−184.9 (m, 1F).

### Synthesis 40

### (E) and (2)-2-(2,2-bis(4-fluorocyclohexyl)vinyl)pyridine (41) and 2-(2-(4-fluorocyclohexyl)-2-(4-fluorocyclohexylidene)ethyl)pyridine (42)

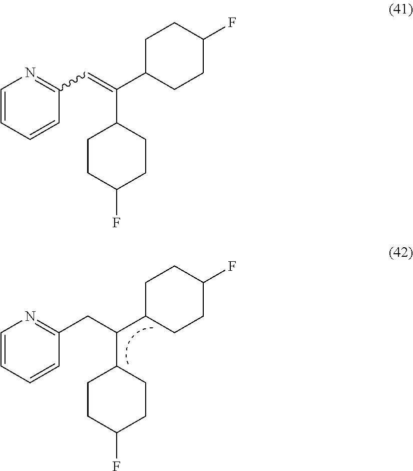 [SMILES: FC1CCC(C(=Cc2ccccn2)C2CCC(F)CC2)CC1 ; FC1CCC(C(Cc2ccccn2)C2CCC(F)CC2)CC1]

To a solution of 2-picoline (7 μL, 0.071 mmol) in THF (2 mL) was added a solution of n-BuLi (25 μL, 0.0625 mmol, 2.5 M in hexanes) at −78° C. The resulting brick-red solution was warmed to −20° C. and stirred for 10 minutes before it was cooled to −78° C. affording a lithiated 2-picoline solution. A THF (1 mL) solution of ketone 40 (10 mg, 0.043 mmol) was cannulated to the lithiated 2-picoline solution at −78° C. and stirred for 30 minutes prior to adding SOCl₂ (6 μL, 0.078 mmol). The reaction mixture was warmed to 23° C. and stirred for 0.5 hours before it was quenched with a saturated solution of NaHCO₃ (5 mL). The resulting mixture was extracted with CH₂Cl₂ (3×10 mL), combined organic layers were dried over Na₂SO₄ and concentrated in vacuo. The residue was purified by flash column chromatography (silica gel, hexanes:EtOAc 10:1) to obtain a mixture of compounds 41 and 42 (11 mg, 88%) as a pale yellow oil.

m/z (ESI) 306.2 [M+H]⁺.

### Synthesis 41

### 2-(2,2-Bis(4-fluorocyclohexyl)ethyl)piperidine (43)

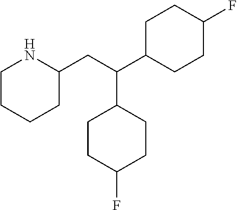 [SMILES: FC1CCC(C(CC2CCCCN2)C2CCC(F)CC2)CC1]

10% Palladium on activated charcoal (5 mg) was suspended in a solution of the crude mixture of 41 and 42 in HOAc (5 mL) at 50° C. under an atmosphere of hydrogen (1 atm) for 24 hours. Then, an equal amount of 10% palladium on activated charcoal was added to the reaction mixture and stirred for further 48 hours at 50° C. before it was filtered through a pad of Celite®. The filtrate was diluted with H₂O (20 mL) and the pH value of the aqueous solution was adjusted to 10 with NaHCO₃ prior to extraction of the solution with CH₂Cl₂ (5×10 mL). The combined organic layers were dried over Na₂SO₄ and concentrated in vacuo. The residue was purified by flash column chromatography (silica gel, NH₄OH_{aq}:MeOH:CH₂Cl₂=2:10:88, v/v) to obtain compound 43 as a pale yellow oil.

¹H NMR (400 MHz, CDCl₃) δ 4.84 (dm, J=48.3 Hz, 1H), 4.45 (dm, J=48.3 Hz, 1H), 3.58 (br s, 1H, NH), 3.22-3.10 (m, 1H), 2.76-2.62 (m, 1H), 2.58-2.40 (m, 1H), 2.21-1.93 (m, 4H), 1.89-1.57 (m, 11H), 1.55-0.86 (m, 12H); ¹⁹F NMR (376 MHz, CDCl₃) δ=−169.1 (dm, J=48.5 Hz, 1F), −184.9 (dm, J=48.5 Hz, 1F); m/z (ESI) 314.2 [M+H]+.

### Biological Methods

### Human and Rat Liver Microsomal Stability Assay

The metabolic stability of fluoroperhexiline derivatives was measured by determination of the rate of compound disappearance when incubated in the presence of both human and rat liver microsomes. Liver microsomes are prepared from the endoplasmic reticulum of hepatocytes and are the primary source of the most important enzymes (cytochrome P450) involved in drug metabolism. Study of drug stability in the presence of liver microsomes is accepted as a valuable model permitting rapid prediction of in vivo drug stability.

### Protocol Summary:

Human and rat liver microsomes were obtained from a commercial source. Test compounds (3 μM) were incubated with pooled liver microsomes (male and female). Samples were incubated for a 45 minute period and removed at 5 time points and test compounds were analysed by LC-MS/MS.

Microsomes (final protein concentration 0.5 mg/mL), 0.1 M phosphate buffer pH 7.4, and test compound (final concentration 3 μM; diluted from 10 mM stock solution to give a final DMSO concentration of 0.25%) were incubated at 37° C. prior to the addition of NADPH (final concentration 1 mM) to initiate the reaction. The final incubation volume was 25 μL. A control incubation was included for each compound tested, where 0.1 M phosphate buffer pH 7.4 was added instead of NADPH. The control compounds testosterone and 7-hydroxycoumarin were included in each experiment and all incubations were performed singularly for each compound.

Each compound was incubated for 0, 5, 15, 30, and 45 minutes. The control (minus NADPH) was incubated for 45 minutes only. The reactions were stopped by the addition of 50 μL methanol containing internal standard at the appropriate time points. The incubation plates were centrifuged at 2500 rpm for 20 minutes at 4° C. to precipitate the protein.

### Quantitative Analysis:

Following protein precipitation, the sample supernatants were combined in cassettes of up to 4 compounds and analysed using standard LC-MS/MS conditions.

### Data Analysis:

From a plot of the natural logarithm of the peak area ratio (i.e., the ratio of compound peak area:internal standard peak area) against time, the gradient of the line was determined. Subsequently, half-life and intrinsic clearance were calculated using the equations below:

Eliminated Rate Constant (k)=(−Gradient).

Half-Life (t_{1/2}) (min)=0.063/k.

Intrinsic Clearance (CLᵢₙₜ) (μL/min/million cells)=(V×0.693)/t_{1/2}.
wherein V=Incubation Volume (μL/mg microsomal protein).

### Cytochrome P450 2D6 Reaction Phenotypinq

The stability of fluoroperhexiline derivatives towards CYP2D6-mediated metabolism was measured by determination of the rate of compound disappearance when incubated in the presence CYP2D6 recombinant isoform expression systems (Bactosomes™). The percentage of parent compound remaining at each time point (following correction for any loss in the incubations with the control bactosomes) is calculated.

### Experimental Procedure:

cDNA expressed human CYP450 enzyme preparations co-expressed with human NADPH cytochrome P450 reductase (Bactosomes™) are supplied by Cypex Ltd. Bactosomes™ are stored at −80° C. prior to use.

Bactosomes™ (final P450 concentration, CYP2D6, 50 pmol/mL), 0.1 M phosphate buffer pH 7.4 and test compound (final substrate concentration=5 μM; final DMSO concentration=0.25%) are pre-incubated at 37° C. prior to the addition of NADPH (final concentration=1 mM) to initiate the reaction. Incubations are also performed using control bactosomes (no P450 enzymes present) to reveal any non-enzymatic degradation. The final incubation volume is 25 μL. A compound known to be metabolised specifically by CYP2D6 isoform is used as a control compound.

Each compound is incubated singly for 0, 5, 15, 30 and 45 min with each isoform. The reactions are stopped by the addition of 50 μL methanol containing internal standard at the appropriate time points. The incubation plates are centrifuged at 2500 rpm for 20 min at 4° C. to precipitate the protein.

### Quantitative Analysis:

Following protein precipitation, the sample supernatants are combined in cassettes of up to four compounds and analysed using Cyprotex generic LC-MS/MS conditions.

### Data Analysis:

The natural logarithm of the peak area ratio (which has been corrected for any loss in the incubations with the control bactosomes) is plotted against time and the gradient of the line determined.

Elimination Rate Constant (k)=(−Gradient).

Half-Life (t_{1/2}) (min)=0.063/k.

### Pharmacokinetics Studies

Absorption, distribution and metabolic stability were studied using an in vivo pharmacokinetics assay. Drug levels were assessed using LC/MS-MS.

Healthy male BALB/c mice (8-12 weeks old) weighing between 25 and 35 g, or Sprague-Dawley rats weighing between 250 and 300 g were procured from In-vivo Bioscience Bangalore, India. Three mice were housed in each cage. Temperature and humidity were maintained at 22±3° C. and 40-70%, respectively and illumination was controlled to give a sequence of 12 h light and 12 h dark cycle. Temperature and humidity were recorded by auto-controlled data logger system. All of the animals were provided laboratory rodent diet (Vetcare India Pvt. Ltd, Bengaluru) after the drug administration. Reverse osmosis water treated with ultraviolet light was provided ad libitum.

### Mouse Studies:

FPER-001 and perhexiline samples were freshly prepared each day before administration. The weighed quantity (11.19 mg) of FPER-001 for p.o. dosing was added into a mortar. A volume of 12 μL of Tween 80 was added and triturated with slow addition of 11.18 mL 0.5% NaCMC in water until it resulted in a homogenous suspension and vortexed for 2 min. The formulation was sonicated for 2 min to obtain a homogenous suspension. Thirty mice were administered with the oral formulation at 10 mg/kg dose through oral gavage using a 22-G oral feeding needle. The dosing volume administered was 10 mL/kg.

### Rat Studies:

FPER-001 and perhexiline samples were freshly prepared each day before administration. The weighed quantity (36.5 mg) of FPER-001 for p.o. dosing was added into a mortar. A volume of 35 μL of Tween 80 was added and triturated with slow addition of 34.625 mL 0.5% NaCMC in water until it resulted in homogenous suspension and vortexed for 2 min. The formulation was sonicated for 2 min to obtain a homogenous suspension.

### Sample Collection:

Blood samples (approximately 60 μL) were collected from retro-orbital plexus of three mice at each time point (1, 4, 8, 24, 48, 72, 96, 120, 144, and 168 h) or of three rats at each time point (0.25, 0.5, 1, 2, 4, 8, 24, 25, 48, 72 and 73 h). Samples were collected into labeled micro-tubes, containing K₂EDTA solution (20% K₂EDTA solution) as an anticoagulant. Plasma was immediately harvested from the blood by centrifugation at 4000 rpm for 10 min at 4±2° C. and stored below −70° C. until bioanalysis. Immediately after collection of blood, myocardium samples were collected from each mouse at 1, 4, 8, 24, 48, 72, 96, 120, 144 and 168 h, or from each rat at 1, 8, 25 and 73 h. Myocardium samples were homogenized using ice-cold phosphate buffer saline (pH 7.4) and homogenates were stored below −70° C. until analysis. Total homogenate volume was five times the myocardium weight in mice, and three times the myocardium weight in rats.

### Analytical Methods:

### Extraction Procedure:

The extraction procedures for plasma/myocardium samples and the spiked plasma calibration standards/quality control samples were identical.

A 25 μL of study sample or spiked calibration standard was added to individual pre-labeled micro-centrifuge tubes followed by 100 μL of internal standard prepared in acetonitrile (Glipizide 500 ng/mL) was added except for the blank, where 100 μL of acetonitrile was added. Samples were vortexed for 5 min. Samples were centrifuged for 10 min at a speed of 4000 rpm at 4° C. Following centrifugation, 100 μL of clear supernatant was transferred in 96-well plates and analysed using LC-MS/MS.

### Bioanalytical Analysis:

HPLC and MS conditions were as follows:

| Chromatographic Mode | LC/MS/MS |
|---|---|
| MS System Used | AB Sciex API-4000 (Q-Trap) |
| Software Version | Analyst 1.5 |
| Scan Type | MRM |
| Polarity | Positive |
| Ion Source | Turbospray |
| Mobile Phase | A: 0.1% Formic acid in acetonitrile |
| | B: 0.1% Formic acid in water |
| Flow Rate (mL/min) | 1.00-1.20 |
| Draw Speed | 200 |
| Splitter | Approximately 50% Out |
| Probe Position | 5 mm vertical, and 5 horizontal |
| Injection Volume (μL) | 5 |
| Auto sampler Temperature (° C.) | 4 |
| Column Oven Temperature (° C.) | 40 |
| Column Used (length × width in | Phenomenex Kinetex KB-C18 |
| mm, particle size) | (150 × 4.6, 100 A) |
| Retention Time (min) | 2.76 |
| | Glipizide-2.86 |
| Run Time (min) | 4.80 |

The HPLC gradient used was:

| Time | Flow | Pump A | Pump B |
|---|---|---|---|
| (min) | (mL/min) | (% Conc) | ( % Conc) |
| 0.01 | 1.0 | 30 | 70 |
| 0.50 | 1.0 | 30 | 70 |
| 1.00 | 1.2 | 90 | 10 |
| 3.00 | 1.2 | 90 | 10 |
| 3.40 | 1.0 | 30 | 70 |
| 4.80 | 1.0 | 30 | 70 |

Pump A = 0.1% Formic acid in acetonitrile.
Pump B = 0.1% Formic acid in water.

The MRM transitions used were:

| Q1 | Q3 | | Dwell | | | |
|---|---|---|---|---|---|---|
| Mass | Mass | | time | | | |
| (Da) | (Da) | I.D. | (msec) | DP | CE | CXP |
| 278.3 | 182.3 | PX001_182 | 80 | 80 | 31 | 8 |
| 446.3 | 347.0 | GLIPIZIDE | 100 | 40.00 | 22.00 | 12.00 |

Source Parameters

| | |
|---|---|
| CAD | Medium |
| CUR | 25 |
| GS1 | 50 |
| GS2 | 50 |
| Ion Spray Voltage | 4500 |
| Temperature | 550 |
| Interface Heater | ON |
| EP | 10 |

### Calculations:

Non-Compartmental-Analysis tool of Phoenix WinNonlin® (Version 6.3) was used to assess the pharmacokinetic parameters. Peak plasma concentrations (Cₘₐₓ) and time for the peak plasma concentrations (Tₘₐₓ) were the observed values. The areas under the concentration time curve (AUCₗₐₛₜ and AUC_{inf}) were calculated by linear trapezoidal rule. The terminal elimination rate constant (kₑ) was determined by regression analysis of the linear terminal portion of the log plasma concentration-time curve.

### Carnitine Palmitoyl Transferase (CPT) Inhibition Assay

Male Sprague-Dawley rats (8-10 weeks of age) were purchased from Charles River and maintained for 10 days under standard housing and feeding conditions.

### Isolation of Rat Heart Mitochondria:

All isolation procedures were performed on ice using pre-cooled solutions and instruments. Rats (n=4 per mitochondrial isolation batch) were sacrificed by CO₂ asphyxiation, and hearts were removed and rinsed in 3 changes of ice-cold phosphate buffered saline (PBS) to remove blood. Hearts were finely minced with scissors, and homogenised in 5 volumes/wt of a buffer containing 300 mM sucrose, 5 mM MOPS, 1 mM EGTA, 5 mM K₂HPO₄, 0.1% BSA, pH 7.4, using a teflon Potter homogenizer. The homogenate was centrifuged at 1500×g for 10 min, the supernatant was collected and centrifuged at 9800×g for 5 min, the mitochondrial pellet was washed and centrifuged twice in the same buffer, and then re-suspended in Assay Buffer (150 mM sucrose, 60 mM KCl, 25 mM Tris/HCl, 1 mM EDTA, 0.1 mM 4,4′-dithiopyridine, 1.3 mg/mL lipid-depleted BSA, pH 6.8). Mitochondrial suspensions were kept on ice and used fresh for each set of experiments.

### Establishment of the M-CPT-1 Assay:

A spectrophotometric assay for M-CPT-1 was used, which measured the decrease in palmitoyl CoA concentration as it was utilised by the enzyme to produce palmitoylcarnitine, according to the following reaction:

palmitoyl CoA+L-carnitine→palmitoylcarnitine+CoA

The disappearance of palmitoyl CoA was monitored as a loss of UV absorbance at 324 nm.

Enzyme reactions (1.0 mL final volume, in a spectrophotometer cuvette) contained:
890 μL of Assay Buffer (see above).
50 μL of mitochondrial suspension, suitably diluted in Assay Buffer.
20 μL of 50-fold concentrated inhibitor in suitable vehicle, or vehicle alone.
20 μL of 2 mM palmitoyl CoA (final concentration 40 μM).
20 μL of 20 mM L-carnitine (final concentration 400 μM).

Samples were pre-incubated with inhibitor for 2 min at 37° C., the palmitoyl CoA was added, and reactions were initiated with the addition of L-carnitine. Tubes were incubated at 37° C., and UV absorbance was recorded for 5 min. The dilution of the mitochondrial suspension was adjusted so as to provide no more than 50% conversion of palmitoyl CoA into palmitoylcarnitine within the assay incubation time in the absence of inhibitor. The protein concentration of the mitochondrial suspension was determined using the Bradford assay reagent.

### Analogue Testing:

Compounds were tested at nine different concentrations (0.1, 0.3, 1, 3, 10, 30, 100, 300 and 1000 μM), plus vehicle blank control. In addition to the FPER compounds, perhexiline and malonyl CoA were tested in parallel as positive control M-CPT-1 inhibitors. Each assay (from a given freshly prepared batch of mitochondria) included triplicate determinations of enzyme activity at each concentration of each inhibitor. The inhibitory potency of each analogue, expressed as the IC₅₀ (the concentration giving 50% inhibition of M-CPT-1 activity under the assay conditions used) was determined by non-linear regression of the log concentration-inhibition curves. The assay itself was replicated on 5 different batches of mitochondria, giving 5 separate estimates of IC₅₀ for each test compound. The mean (+/−SD, n=5) IC₅₀ for each test compound and for perhexiline and malonyl CoA was reported.

### Biological Data

### Biological Study 1

The metabolic stability of a number of fluoroperhexiline (FPER) compounds was determined and compared with the metabolic stability of the parent perhexiline molecule, using the assays described above.

Biological half-life values (t_{1/2}) were determined for several fluoroperhexiline compounds, as well as perhexiline itself, using the human liver microsomal (HLM) and rat liver microsomal (RLM) stability assays described above. The results are summarised in the following table.

| | | CLᵢₙₜ | | CLᵢₙₜ | |
|---|---|---|---|---|---|
| | | (μL/min/ | T½ | (μL/min/ | T½ |
| | | mg protein) | (min) | mg protein) | (min) |
| Compound | Structure | HLM | HLM | RLM | RLM |
| Perhexiline | 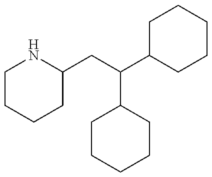 [SMILES: C1CCC(C(CC2CCCCN2)C2CCCCC2)CC1] | 13.8 | 100 | 65 | 21 |
| FPER-001 | 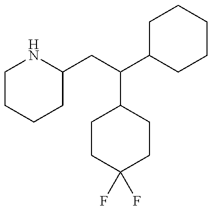 [SMILES: FC1(F)CCC(C(CC2CCCCN2)C2CCCCC2)CC1] | 2.6 | 525 | 19 | 72 |
| FPER-002 | 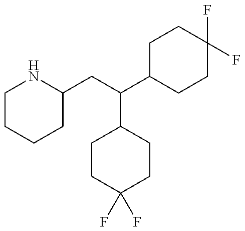 [SMILES: FC1(F)CCC(C(CC2CCCCN2)C2CCC(F)(F)CC2)CC1] | 0.3 | 4210 | 0.8 | 1650 |
| FPER-003 | 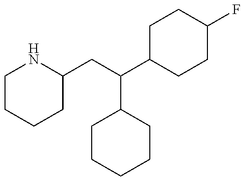 [SMILES: FC1CCC(C(CC2CCCCN2)C2CCCCC2)CC1] | 21.3 | 65 | 129 | 11 |

These data demonstrate that replacing both of the hydrogen atoms at the para-position of one or both of the cyclohexyl groups of perhexiline with fluorine atoms (as in, e.g., FPER-001 and FPER-002) provides a substantial increase in metabolic stability.

These data also demonstrate that this substitution does not give results which could have been predicted with reasonable certainty, for example, because the replacement of only one hydrogen atom with a fluorine group (as in, e.g., FPER-003) gives a substantial decrease in stability.

These data demonstrate that the FPER compounds have improved metabolic stability as compared to perhexiline and thus have advantages over perhexiline, such as less frequent dosing, reduced formation of toxic metabolites, and lower doses, which may in turn cause fewer side-effects.

### Biological Study 2

The stability in the presence of CYP2D6 in a number of FPER compounds was determined and compared with the metabolic stability of the parent perhexiline molecule using the assays described previously.

Biological half-life values (t_{1/2}) were determined for several FPER compounds, as well as perhexiline itself, using the Cytochrome P450 2D6 Reaction Phenotyping assay described above. The results are summarised in the following table.

| Compound | Structure | T½ (min) |
|---|---|---|
| Perhexiline | 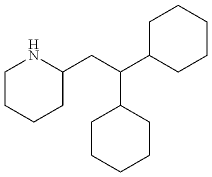 [SMILES: C1CCC(C(CC2CCCCN2)C2CCCCC2)CC1] | 14 |
| FPER-001 | 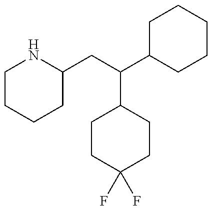 [SMILES: FC1(F)CCC(C(CC2CCCCN2)C2CCCCC2)CC1] | 104 |
| FPER-002 | 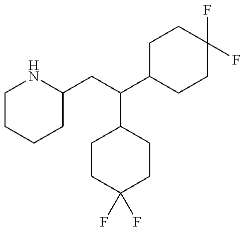 [SMILES: FC1(F)CCC(C(CC2CCCCN2)C2CCC(F)(F)CC2)CC1] | 151 |
| FPER-003 | 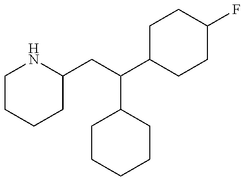 [SMILES: FC1CCC(C(CC2CCCCN2)C2CCCCC2)CC1] | 16 |

These data demonstrate that replacing both of the hydrogen atoms at the para-position of one or both of the cyclohexyl groups of perhexiline with fluorine atoms (as in, e.g., FPER-001 and FPER-002) provides a substantial increase in stability towards CYP2D6.

These data also demonstrate that this substitution does not give results which could have been predicted with reasonable certainty, for example, because replacement of only one hydrogen atom with a fluorine group (as in, e.g., FPER-003) gives no increase in stability.

### Biological Study 3

The oral absorption, distribution, and stability of FPER-001 and perhexiline were compared in the mouse model as described above. Maximum concentration (Cₘₐₓ) and area under the curve (AUC) were investigated for both plasma and myocardia in male BALB/c mice, following 10 mg/kg oral gavage of FPER-001 or perhexiline, using an LC/MS/MS detection system as described above. The results are summarised in the following table.

| | FPER-001 | | Perhexiline | |
|---|---|---|---|---|
| | (10 mg/kg) | | (10 mg/kg) | |
| | Plasma | Myocardium | Plasma | Myocardium |
| Cₘₐₓ (ng/mL) | 346.51 | 1678.38 | 116.35 | 664.53 |
| AUC 0-24 | 3726.75 | 32361.04 | 349.67 | 1827.74 |
| (hr*ng/mL) | | | | |

These data show that there is a significantly higher maximum drug concentration following oral dosing with FPER-001 than with perhexiline, in both plasma (˜3-fold higher) and myocardium (˜2.5-fold higher).

These data also show that there is a much greater drug exposure following oral dosing with FPER-001 than with perhexiline in both plasma (˜11-fold higher) and the myocardium (˜18-fold higher).

### Biological Study 4

The oral absorption, distribution, and stability of FPER-001 and perhexiline were compared in the rat model as described above. Concentration was investigated for both plasma and myocardia in male Sprague-Dawley rats using an LC/MS/MS detection system as described above. The results are summarised in the following table.

| | |
|---|---|
| FPER-001 | Perhexiline |
| (10 mg/kg) | (10 mg/kg) |

| | Time | Plasma | Myocardium | Plasma | Myocardium |
|---|---|---|---|---|---|
| Conc. (ng/mL) | 1 h | 386.30 | 5913.43 | 99.94 | 1901.48 |
| Conc. (ng/mL) | 8 h | 178.63 | 1285.93 | 31.39 | 361.16 |

These data show that there is a significantly higher drug concentration following oral dosing with FPER-001 than with perhexiline, in both plasma (˜4-fold higher at 1 h; ˜6-fold higher at 8 h) and the myocardium (˜3-fold higher at 1 h; ˜3.5-fold higher at 8 h). Thus these data also show that there is a much greater drug exposure following oral dosing with FPER-001 than with perhexiline.

In order for a drug to be therapeutically active, it is necessary not only that it reaches the target tissue in sufficient quantities, but also that a therapeutic concentration is maintained for an extended time period. As FPER-001 reaches and maintains a greater drug concentration in the myocardium than perhexiline, it is more likely to be able to exert a therapeutic effect for the treatment of a cardiovascular condition. Furthermore, because of this selective concentration in the myocardium, FPER-001 is less likely to show off-target toxic effects in other tissues than perhexiline.

These data show that the FPER compounds are much less heavily metabolised by CYP2D6 than perhexiline and thus will not show the inter-individual variability which compromises perhexiline therapy. Therefore, the FPER compounds are expected to show more reliable and predictable pharmacokinetics and hence may avoid the toxicity issues found with perhexiline. As a consequence, the FPER compounds are expected to be suitable for use across a broader spectrum of the patient population and not to require additional monitoring of drug plasma concentrations or patient phenotyping, both of which limit drug use and convenience.

### Biological Study 5

The IC₅₀ for inhibition of cardiac CPT-1 for a number of FPER compounds was determined and compared with that of perhexiline using the assays described above. The data are summarised in the following table.

| Compound | Structure | IC₅₀ μM |
|---|---|---|
| Perhexiline | 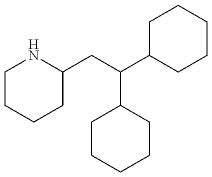 [SMILES: C1CCC(C(CC2CCCCN2)C2CCCCC2)CC1] | 18 |
| FPER-001 | 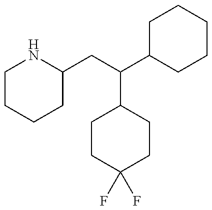 [SMILES: FC1(F)CCC(C(CC2CCCCN2)C2CCCCC2)CC1] | 35 |
| FPER-003 | 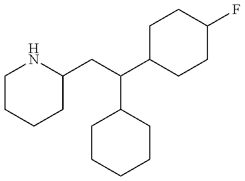 [SMILES: FC1CCC(C(CC2CCCCN2)C2CCCCC2)CC1] | 85 |

As this target, CPT-1, is believed to be responsible for mediating the therapeutic activity of perhexiline, these data indicate that the FPER compounds can be expected to have therapeutic potential in a similar range of disease indications to perhexiline and to show roughly similar efficacy.

These data demonstrate that replacing both of the hydrogen atoms at the para-position of one the cyclohexyl groups of perhexiline with fluorine atoms (as in, e.g., FPER-001) gives a level of inhibition of cardiac CPT-1 that is similar to perhexiline (IC₅₀ of 35 μM as compared to 18 μM).

These data also demonstrate that this substitution does not give results which could have been predicted with reasonable certainty, for example, because the replacement of only one hydrogen atom with a fluorine group (e.g., as in FPER-003) gives a much lower level of inhibition as compared to perhexiline (IC₅₀ of 85 μM as compared to 18 μM).

The foregoing has described the principles, preferred embodiments, and modes of operation of the present invention. However, the invention should not be construed as limited to the particular embodiments discussed. Instead, the above-described embodiments should be regarded as illustrative rather than restrictive. It should be appreciated that variations may be made in those embodiments by workers skilled in the art without departing from the scope of the present invention.

### REFERENCES

A number of publications are cited herein in order to more fully describe and disclose the invention and the state of the art to which the invention pertains. Full citations for these publications are provided below. Each of these publications is incorporated herein by reference in its entirety into the present disclosure, to the same extent as if each individual publication was specifically and individually indicated to be incorporated by reference.
Abozguia et al., 2010, “Metabolic Modulator Perhexiline Corrects Energy Deficiency and Improves Exercise Capacity in Symptomatic Hypertrophic Cardiomyopathy,” Circulation, Vol. 122, pp. 1562-1569.
Ashrafian et al., 2007, “Perhexiline,” Cardiovascular Drug Reviews, Vol. 25, No. 1, pp. 76-97.
Ceccarell et al., 2011, “Carnitine Palmitoyltransferase (CPT) Modulators: A Medicinal Chemistry Perspective on 35 Years of Research,” J. Med. Chem., Vol. 54, pp. 3109-3152.
Cho et al., 1970, “Pharmacology of a New Antianginal Drug: Perhexiline: I. Coronary Circulation and Myocardial Metabolism,” Chest, Vol. 58, pp. 577-581.
Cooper et al., 1987, “Studies on the metabolism of perhexiline in man”, Eur. J. Clin. Pharmacol., Vol. 32, pp. 569-576.
Davies et al., 2006, “Determination of the 4-monohydroxy metabolites of perhexiline in human plasma, urine and liver microsomes by liquid chromatography” J. Chromatog. B, Vol. 843, pp. 302-309.
Davies et al., 2007a, “CYP2B6, CYP2D6 and CYP3A4 catalyze the primary oxidative metabolism of perhexiline enantiomers by human liver microsomes,” Drug Metab. Dispos., Vol. 35, pp. 128-138.
Davies et al., 2007b, “Steady-state pharmacokinetics of the enantiomers of perhexiline in CYP2D6 poor and extensive metabolizers administered Rac-perhexiline,” Brit. J. Clin. Pharmacol., Vol. 65, pp. 347-354.
Fan et al., 2004, “An Efficient Total Synthesis of (±)-Lycoramine,” Org. Lett., Vol. 6, pp. 4691-4694.
Frenneaux, 2002, “New Tricks for an Old Drug,” Eur. Heart J., Vol. 23, pp1898-1899.
Frenneaux, 2012, “Perhexiline for Treating Chronic Heart Failure,” US 2012/0101128 A1, published 26 Apr. 2012.
Fukumura et al., 2003 “Process for producing alpha, alpha-difluorocycloalkane compound,” US patent publication number US 2003/0078460 A1 published 24 Apr. 2003.
Galluzzi et al., 2013, “Metabolic targets for cancer therapy”, Nature Reviews: Drug Discovery, Vol. 12, pp. 829-846.
Glorius et al., 2004, “Efficient Asymmetric Hydrogenation of Pyridines,” Angew. Chem. Int. Ed., Vol. 43, pp. 2850-2852.
Hagmann, 2008, “The Many Roles for Fluorine in Medicinal Chemistry,” J. Med. Chem., Vol. 51, pp. 4359-4369.
Heitbaum et al., 2010, “Diastereoselective Hydrogenation of Substituted Quinolines to Enantiomerically Pure Decahydroquinolines,” Adv. Synth. Catal., Vol. 352, pp. 357-362.
Horgan et al., 1978, “Process for Preparing 2-(2,2-Dicyclohexylethylpiperidine,” U.S. Pat. No. 4,069,222 granted 17 Jan. 1978.
Horgan et al., 1980, “Process for Preparing 2-(2,2-Dicyclohexylethylpiperidine,” U.S. Pat. No. 4,191,828 granted 4 Mar. 1980.
Hu et al., 2006, “Total Synthesis of (±)-Galanthamine,” Org. Lett., Vol. 8, pp. 1823-1825.
Hudak et al., 1970, “Cardiovascular Pharmacology of Perhexiline,” J. Pharmacol. Exp. Ther., Vol. 173, pp. 371-382.
Inglis et al., 2007, “Effect of CYP2D6 metabolizer status on the disposition of the (+) and (−) enantiomers of perhexiline in patients with myocardial ischaemia,” Pharmacogenetics and Genomics, Vol. 17, pp. 305-312.
Kaye and Krum, 2007, “Drug Discovery for heart failure: a new era or the end of the pipeline?” Nat. Rev. Drug Disc., Vol. 6, pp. 127-139.
Kennedy et al., 1996, “Inhibition of carnitine palmitoyltransferase-1 in rat heart and liver by perhexiline and amiodarone,” Biochem. Pharmacol., Vol. 52, pp. 273-280.
Kennedy et al., 1997, “Methods related to the treatment of and isolation of compounds for treatment of ischaemic conditions”, International patent application publication number WO 97/00678 A1 published 9 Jan. 1997.
LeClerc et al., 1982, “Synthesis and Cardiovascular Activity of a New Series of Cyclohexylaralkylamine Derivatives Related to Perhexiline,” J. Med. Chem., Vol. 25, pp. 709-714.
Lee et al., 2005, “Metabolic Modulation With Perhexiline in Chronic Heart Failure A Randomized, Controlled Trial of Short-Term Use of a Novel Treatment,” Circulation, Vol. 112, pp. 3280-3288.
Matthews et al., 2003, “A New method for the electrophilic fluorination of vinyl stannanes,” Tet. Lett., Vol. 19, pp. 3057-3060.
Meanwell, 2011, “Synopsis of Some Recent Tactical Application of Bioisosteres in Drug Design,” J. Med. Chem., Vol. 54, pp. 2529-2591.
Muller et al., 2007, “Fluorine in Pharmaceuticals: Looking Beyond Intuition,” Science, Vol. 317, pp. 1881-1886.
Neubauer, 2007, “The failing heart—an engine out of fuel,” N. Engl. J. Med., Vol. 356, pp. 1140-1151.
Pike et al., 2011, “Inhibition of fatty acid oxidation by etomoxir impairs NADPH production and increases reactive oxygen species resulting in ATP depletion and cell death in human glioblastoma cells” Biochim. Biophys. Acta, Vol. 1807, pp. 726-734.
Sallustio et al., 2002, “Pharmacokinetics of the antianginal agent perhexiline: relationship between metabolic ratio and steady-state dose,” Brit. J. Clin. Pharmacol., Vol. 54, pp. 107-114.
Sallustio et al., 2014, “Uses of (−)-perhexiline”, International patent application publication number WO 2014/036603 A1, published 13 Mar. 2014.
Samudio et al., 2010, “Pharmacologic inhibition of fatty add oxidation sensitizes human leukemia cells to apoptosis induction”, J. Clin. Invest., Vol. 120, pp. 142-156.
Schelper et al., 2004, “Catalysis-based enantioselective total synthesis of the macrocyclic spermidine alkaloid isooncinotine,” Proc. Natl. Acad. Sci. USA, Vol. 101, pp. 11960-11965.
Schou, 2010, “Influence of [²H]-labelled acetic acid as solvent in the synthesis of [²H]-labelled perhexiline,” J. Label. Compd Radiopharm., Vol. 53, pp. 31-35.
Scott and Stille, 1986, “Palladium-Catalyzed Coupling of Vinyl Triflates with Organostannanes. Synthetic and Mechanistic Studies,” J. Am. Chem. Soc., Vol. 108, pp. 3033-3040.
Singlas et al., 1978, “Pharmacokinetics of perhexiline maleate in anginal patients with and without peripheral neuropathy,” Eur. J. Clin. Pharmacol., Vol. 14, pp. 195-201.
Sweitzer and Stevenson, 2000, “Diastolic Heart Failure: Miles to Go Before We Sleep”, Am. J. Med., Vol. 109, pp. 683-685.
Tamargo and Lopez-Sendon, 2011, “Novel Therapeutic Targets for the Treatment of Heart Failure,” Nat. Rev. Drug Disc., Vol. 10, pp. 536-555.
Tassoni et al., 2007, “Inhibitors of CPT In the Central Nervous System as Antidiabetic and/or Anti-obesity Drugs,” international patent publication number WO 2007/096251 A1 published 30 Aug. 2007.
Tilford and van Campen, 1954, “Diuretics. α,α-Disubstituted 2-Piperidine-ethanols and 3,3-Disubstituted Octahydropyrid[1,2-c]oxazines,” J. Am. Chem. Soc., Vol. 76, pp. 2431-2441.
Unger et al., 1997, “Perhexiline improves symptomatic status in elderly patients with severe aortic stenosis,” Aust. N. Z. J. Med., Vol. 27, pp. 24-28.
Vander Heiden, 2011, “Targeting cancer metabolism: a therapeutic window opens”, Nature Reviews: Drug Discovery, Vol. 10, pp. 671-684.
Willoughby et al., 2002, “Beneficial clinical effects of perhexiline in patients with stable angina pectoris and acute coronary syndromes are associated with potentiation of platelet responsiveness to nitric oxide,” Eur. Heart J., Vol. 23, pp. 1947-1954.
Zaugg et al., 2011, “Carnitine palmitoyltransferase 1C promotes cell survival and tumor growth under conditions of metabolic stress”, Genes Dev., Vol, 25, pp. 1041-1051.

1-55. (canceled)

56. A compound of the following formula: 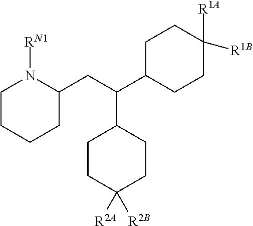
or a pharmaceutically acceptable salt thereof;
wherein:
—R^{1A} is —F;
—R^{1B} is —F;
and wherein:
—R^{2A} is —H; and —R^{2B} is —H; or
—R^{2A} is —F; and —R^{2B} is —F;
and wherein:
—R^{N1} is independently —H, —R^{NN}, or —C(═O)—R^{NNN};
—R^{NN} is saturated linear or branched C₁₋₄alkyl;
—R^{NNN} is independently saturated linear or branched C₁₋₄alkyl, phenyl, or benzyl.

57. A compound according to claim 56, which is a compound of the following formula, or a pharmaceutically acceptable salt thereof:

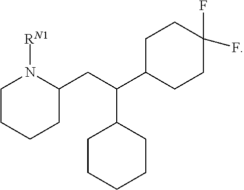

58. A compound according to claim 56, which is a compound of the following formula, or a pharmaceutically acceptable salt thereof:

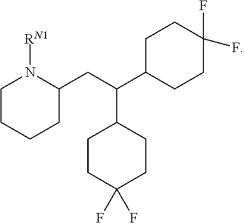

59. A compound according to claim 56, wherein —R^{N1} is independently —H or —R^{NN}.

60. A compound according to claim 57, wherein —R^{N1} is independently —H or —R^{NN}.

61. A compound according to claim 58, wherein —R^{N1} is independently —H or —R^{NN}.

62. A compound according to claim 56, wherein —R^{N1} is —H.

63. A compound according to claim 57, wherein —R^{N1} is —H.

64. A compound according to claim 58, wherein —R^{N1} is —H.

65. A compound according to claim 56, which is selected from compounds of the following formulae, and pharmaceutically acceptable salts thereof:

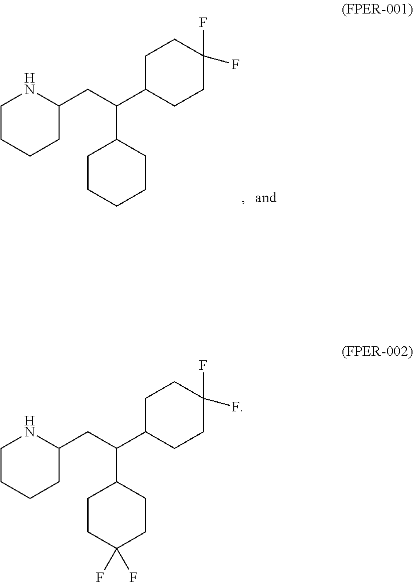 [SMILES: FC1(F)CCC(C(CC2CCCCN2)C2CCCCC2)CC1 ; FC1(F)CCC(C(CC2CCCCN2)C2CCC(F)(F)CC2)CC1]

66. A composition comprising a compound according to claim 56, and a pharmaceutically acceptable carrier or diluent.

67. A method of preparing a composition comprising the step of mixing a compound according to claim 56 and a pharmaceutically acceptable carrier or diluent.

68. A method of inhibiting carnitine palmitoyltransferase (CPT), in vitro or in vivo, comprising contacting the CPT with an effective amount of a compound according to claim 56.

69. A method of treatment of a disorder comprising administering to a patient in need of treatment a therapeutically effective amount of a compound according to claim 56, wherein the disorder is:
a disorder that is ameliorated by the inhibition of carnitine palmitoyltransferase (CPT);
a disorder that is ameliorated by inhibition of fatty acid oxidation;
a disorder that is characterised by impaired cardiac energetics; or
a disorder that is characterised by oxygen deficiency.

70. A method of treatment of a disorder comprising administering to a patient in need of treatment a therapeutically effective amount of a compound according to claim 56, wherein the disorder is ischaemia.

71. A method of treatment of a disorder comprising administering to a patient in need of treatment a therapeutically effective amount of a compound according to claim 56, wherein the disorder is a cardiovascular disorder.

72. A method of treatment of a disorder comprising administering to a patient in need of treatment a therapeutically effective amount of a compound according to claim 56, wherein the disorder is:
angina pectoris; heart failure (HF); left or right ventricular failure; pulmonary heart disease; ischaemic heart disease (IHD); cardiomyopathy; cardiac dysrhythmia; stenosis of a heart valve; hypertrophic cardiomyopathy (HCM); or coronary heart disease.

73. A method of treatment of a disorder comprising administering to a patient in need of treatment a therapeutically effective amount of a compound according to claim 56, wherein the disorder is:
angina pectoris caused by coronary heart disease; angina pectoris caused by ischaemia; severe angina pectoris; or unresponsive or refractory angina pectoris;
heart failure caused by ischaemia; congestive heart failure; chronic heart failure; moderate heart failure; systolic heart failure; diastolic heart failure; or diastolic heart failure with left ventricular injury;
left or right ventricular failure;
pulmonary heart disease caused by pulmonary hypertension; pulmonary heart disease caused by chronic obstructive lung disease; or pulmonary heart disease caused by emphysema;
ischaemic heart disease caused by coronary heart disease; ischaemic heart disease caused by obstruction of the coronary artery; ischaemic heart disease caused by spasm of the coronary artery; severe ischaemic heart disease; or refractory ischaemic heart disease;
cardiomyopathy due to ischaemic heart disease or cardiomyopathy due to hypertension;
cardiac dysrhythmia caused by ischaemia;
aortic stenosis;
symptomatic non-obstructive hypertrophic cardiomyopathy; or
coronary heart disease.

74. A method of treatment of a disorder comprising administering to a patient in need of treatment a therapeutically effective amount of a compound according to claim 56, wherein the disorder is:
diabetes; hyperglycemia; hyperlipidemia; hypertriglyceridemia; dyslipidemia; syndrome X; or obesity.

75. A method of treatment of a disorder comprising administering to a patient in need of treatment a therapeutically effective amount of a compound according to claim 56, wherein the disorder is:
acute myeloid leukaemia; adrenal gland cancer; biliary tract cancer; bladder cancer; bone cancer; bowel cancer; brain cancer; breast cancer; colon cancer; colorectal cancer; endometrial cancer; gastrointestinal cancer; genito-urinary cancer; glioma; glioblastoma; gynaecological cancer; head cancer; Hodgkin's disease; Kaposi's sarcoma; kidney cancer; large bowel cancer; leukaemia; liver cancer; lung cancer; lymphoma; lymphocytic leukaemia (lymphoblastic leukaemia); malignant melanoma; mediastinum cancer; melanoma; myeloma; myelogenous leukaemia (myeloid leukaemia); nasopharyngeal cancer; neck cancer; nervous system cancer; non-Hodgkin's lymphoma; non-small cell lung cancer; oesophagus cancer; osteosarcoma; ovarian cancer; pancreatic cancer; prostate cancer; rectal cancer; renal cell carcinoma; sarcoma; skin cancer; small bowel cancer; small cell lung cancer; soft tissue sarcoma; squamous cancer; stomach cancer; testicular cancer; or thyroid cancer.
